(19) United States
(12) Patent Application Publication
(10) Pub. No.: US 2026/0240870 A1
(43) Pub. Date: Aug. 20, 2026

(54) METHODS OF REDUCING TRANSPLANT REJECTION IN SUBJECTS UNDERGOING IMMUNOSUPPRESSANT THERAPY

(71) Applicant: University of Maryland, Baltimore, Baltimore, MD (US)

(72) Inventors: Bing Ma, Clarksville, MD (US); Jonathan S. Bromberg, Baltimore, MD (US)

(21) Appl. No.: 19/543,131

(22) Filed: Feb. 18, 2026

Related U.S. Application Data
(60) Provisional application No. 63/759,713, filed on Feb. 18, 2025.
(60) Provisional application No. 63/824,523, filed on Jun. 16, 2025.

(51) Int. Cl. A61K 31/573; A61K 31/436; A61K 31/5355; A61K 35/745; A61K 35/747; A61P 37/00; C12Q 1/04; G01N 33/569

(52) U.S. Cl. CPC A61K 31/573; A61K 31/436; A61K 31/5355; A61K 35/745; A61K 35/747; A61P 37/00; C12Q 1/04; G01N 33/569

(57) ABSTRACT

The present invention provides a method of reducing transplant rejection in a subject undergoing an immunosuppressant therapy, comprising detecting a dysbiotic state in the subject's gut microbiota resulting from the immunosuppressant therapy; and administering a therapy that attenuates the dysbiotic state in the subject's gut microbiota.

### CROSS-REFERENCE TO RELATED APPLICATIONS

This application claims the benefit of U.S. Application No. 63/759,713, filed Feb. 18, 2025 and U.S. Application No. 63/824,523, filed on Jun. 16, 2025, the contents of which are incorporated by reference in their entirety.

### STATEMENT REGARDING FEDERALLY SPONSORED RESEARCH AND DEVELOPMENT

This invention was made with government support under the Grant Numbers AI170050, HL148672, and AI114496 awarded by the National Institutes of Health. The government has certain rights in the invention.

### INCORPORATION-BY-REFERENCE OF MATERIAL SUBMITTED

LENGTHY TABLES
The patent application contains a lengthy table section. A copy of the table is available in electronic form from the USPTO web site (https://seqdata.uspto.gov/docdetail?docId=US20260240870A1). An electronic copy of the table will also be available from the USPTO upon request and payment of the fee set forth in 37 CFR 1.19(b)(3).

Incorporated by reference in its entirety herein is a computer-readable sequence listing submitted concurrently herewith and identified as follows: One 2,886 Byte XML file named “Sequence_listing.xml,” created on Feb. 18, 2025.

Incorporated by reference in its entirety herein is a computer-readable Table 1 submitted concurrently herewith and identified as follows: One 47,016 Byte text file named “Table_1.txt,” created on Feb. 18, 2025.

Incorporated by reference in its entirety herein is a computer-readable Table 2 submitted concurrently herewith and identified as follows: One 63,517 Byte text file named “Table_2.txt,” created on Feb. 18, 2025.

Incorporated by reference in its entirety herein is a computer-readable Table 3 submitted concurrently herewith and identified as follows: One 46,777 Byte text file named “Table_3.txt,” created on Feb. 18, 2025.

Incorporated by reference in its entirety herein is a computer-readable Table 4 submitted concurrently herewith and identified as follows: One 53,458 Byte text file named “Table_4.txt,” created on Feb. 18, 2025.

### FIELD OF THE INVENTION

The present invention generally relates to the fields of medicine, particularly transplant surgery and treatment, microbiology and pharmaceuticals.

### BACKGROUND OF THE INVENTION

Prevention of solid organ transplant rejection necessitates lifelong, multimodal immunosuppression to dampen both adaptive and innate alloreactive immunity. Despite improved short-term outcomes, long-term graft survival remains suboptimal due to chronic rejection and a range of adverse events such as metabolic complications, infections, neoplasia, and organ-specific toxicities (Lodhi et al., American Journal of Transplantation, (2011), 11:1226-1235; Dai et al., Frontiers in Immunology, (2020), 11:10.3389/fimmu.2020.01044; Fishman et al., Am J Transplant, (2017), 17:856-879; Gallagher et al., J Am Soc Nephrol, (2010), 21:852-858; Schmitz et al., Nephron Exp Nephrol, (2009), 111:e80-91; Gioco et al., World J Gastroenterol, (2020), 26, 5797-5811; Wijdicks, E. F., Liver Transpl, (2001), 7:937-942; Bhat et al., Endocr Rev, (2021), 42:171-197). Current immunosuppressants target specific immune pathways but are limited by their antigen-non-specific nature, broadly suppressing the immune system and causing off-target effects (Nayak et al., J Biosci, (2022), 47:10.1007/s12038-022-00312-4; Xie et al., J Release, (2020), 328:237-250). This dilemma between necessary Control immunosuppression and its imprecise, system-wide effects represents a critical challenge for improving long-term transplant outcomes.

A comprehensive understanding of the effects of immune suppressant drugs is essential for optimizing therapeutic regimens, minimizing adverse events, and preventing graft rejection. While most mechanistic studies on immunosuppressants have focused on their effects on T cells, B cells, and dendritic cells (DCs), little is known about their impact on secondary lymphoid organs (SLOs), particularly lymph nodes (LNs). As key regulators of local and systemic immunity, LNs direct immune responses toward tolerance or activation, with their function critically dependent on the architecture maintained by lymph node stromal cell (LNSC) networks, including fibroblastic reticular cells (FRCs), lymphatic endothelial cells (LECs), and blood endothelial cells (BECs). LNSCs orchestrate the positioning and interaction of lymphocytes with antigen-presenting cells (APCs) like DCs through the coordinated action of chemokines, cytokines, and stromal fibers (Saxena et al., Immunol Rev, (2019), 292:9-23; Li et al., Trends Immunol, (2021), 42:723-734; Mueller et al., Science, (2007), 317:670-674). FRC-derived laminins are particularly important, as the laminin α4:α5 ratio (La4:La5) within the T cell cortex of the cortical ridge (CR) and around high endothelial venules (HEVs) modulates the balance between tolerance and immunity and influences global immune states (Li, L., Shirkey et al., J Clin Invest, (2020), 130:2602-2619; Simon et al., Transplantation, (2019), 103:2075-2089). An increased La4:La5 promotes a pro-tolerogenic environment, whereas a decreased ratio fosters inflammation. LN structure and FRC function are often disrupted during transplantation due to ischemia-reperfusion injury (IRI) to the graft, leading to persistent “immunologic scarring”, a pathologic alteration of the LN cellular network (Maarouf et al., JCI Insight, (2018), 3:10.1172/jci.insight. 120546; Mongodin et al., Curr Opin Organ Transplant, (2021), 26:567-581). Further, regulatory T cells (Tregs) do not simply suppress immune responses through their presence alone. Their precise localization within LNs is critical for maintaining immune homeostasis. Tregs around HEVs limit the entry of pro-inflammatory effector T cells into the LNs. This gatekeeping function helps maintain a tolerogenic environment by preventing excessive accumulation of pro-inflammatory cells (Li, L., Shirkey et al., J Clin Invest, (2020), 130:2602-2619). Tregs in the CR suppress T cell priming and activation by APCs, preventing effector T cell activation and differentiation (Scott et al., Front Immunol, (2021), 12:702726). A reduction of Tregs in these regions can result in enhanced T cell activation, promoting a pro-inflammatory state (Lu et al., Transpl Immunol, (2021), 67:101411; Warren et al., J Clin Invest, (2014), 124:2204-2218; Anggelia et al., Transplantation, (2021), 105:1238-1249). Despite the importance of LN architecture and Treg localization in immune modulation, the effects of immunosuppressive drugs on these structural and functional elements remain poorly understood. Addressing this gap is critical for developing strategies to enhance long-term allograft survival.

Beyond their immunomodulatory effects, immunosuppressive drugs profoundly influence gut microbiome composition and function, which has been significantly implicated in alloimmunity and graft survival (Lei et al., J Clin Invest, (2016), 126:2736-2744; Hossain et al., J Immunol, (2011), 187:5130-5140; Jenq et al., J Exp Med, (2012), 209:903-911; Oh et al., Am J Transplant, (2012), 12:753-762; Willner et al., Am J Respir Crit Care Med, (2013), 187:640-647; Kensiski et al., Clin Microbiol Rev, (2025), e0017824). Our group has demonstrated that gut microbiota modulates LN architecture by altering stromal fiber laminins (Koren et al., Cell, (2012), 150:470-480; Rooks et al., The ISME journal, (2014), 8:1403-1417; Bromberg, et al., JCI Insight, (2018), 3:10.1172/jci.insight.121045). Our recent studies of tacrolimus and the mammalian target of rapamycin (mTOR) inhibitor rapamycin have revealed broad effects of these drugs on gut microbiome, luminal metabolic functions, intestinal transcriptome, as well as LN architecture, consistent with previous studies (Wu et al., JCI Insight, (2025), 10:10.1172/jci.insight. 186505; Ma et al., BMC Microbiol, (2023), 23:394; Gao et al., Int J Mol Sci, (2023), 24:10.3390/ijms241411811; Liu et al., Front Pharmacol, (2018), 9:1520). Other research has shown diverse mechanisms by which immunosuppressants affect host-microbe interactions. For example, methotrexate inhibits the conserved dihydrofolate reductase pathway in both host and gut bacteria, leading to broad microbiome alterations, while certain immunosuppressants disrupt the intestinal barrier, altering intestinal permeability (Nayak et al., Cell Host Microbe, (2021), 29:362-377; Khan et al., Frontiers in physiology, (2017), 8:438). Notably, these host-microbe interactions are bidirectional, as gut microbiota can also alter intestinal function and drug bioavailability (Wilson et al., Transl Res, (2017), 179:204-222; Haiser et al., Science, (2013), 341:295-298). For example, mycophenolate mofetil (MMF) is metabolized to inactive glucuronidated mycophenolic acid (MPAG) via hepatic glucuronidation, and luminal bacteria expressing β-glucuronidase can reactivate the metabolized form MPAG back to active MPA, leading to increased concentrations of active luminal drug and colonic inflammation (Taylor et al., Sci Adv, (2019), 5:eaax2358). These complex interactions among immunosuppressants, gut microbiota, intestinal function, and immune cell populations underscore the need for a comprehensive understanding of their interrelationships to optimize therapeutic efficacy and mitigate unintended effects.

The foregoing description of the background is provided to aid in understanding the invention, and is not admitted to be or to describe prior art to the invention.

### SUMMARY OF THE INVENTION

It is to be understood that both the foregoing general description of the invention and the following detailed description are exemplary, and thus do not restrict the scope of the invention.

Current immunosuppressants effectively suppress adaptive and innate immune responses, but their broad, antigen-non-specific effects often result in significant complications. The present disclosure describes an investigation of the immunosuppressant effects of four major immunosuppressant classes, including tacrolimus, prednisone, mycophenolate mofetil (MMF), and fingolimod (FTY), on the gut microbiome, metabolic pathways, lymphoid architecture and lymphocyte trafficking after up to 30-day chronic exposure. Despite their distinct mechanisms of action and not designed to target the gut, all immunosuppressive drugs induced profound and time-dependent alterations in both intestine gene expression and gut microbiome composition. Progressive alterations from moderate early, drug-specific changes to a strikingly convergent microbial dysbiosis, marked by significant expansion of pathobionts of Muribaculaceae, occurred across all drug classes. Concurrently, all drugs uniformly induced significant suppression of mucosal immunity including B cell, immunoglobulin, and antigen recognition. Time-dependent changes in lymph node (LN) reorganization and cellular composition were also observed, marked by a progressive shift toward pro-inflammatory phenotypes in gut-draining mesenteric LNs and a gradual loss of tolerogenic architecture in peripheral LNs. Drug-specific metabolic alterations and distinct phases of intestinal transcriptional responses were also characterized. Notably, MMF and FTY demonstrated the most robust immunomodulatory properties, and were able to suppress alloantigen-induced inflammation through mediating regulatory T cell distribution and LN remodeling. Together, these findings highlight the underappreciated complexity and temporal dynamics induced by immunosuppressants, particularly in the gut and compartmentalized alloimmune regulation in lymphoid tissues. Understanding these relationships offers new opportunities for refining immunosuppressive strategies to mitigate treatment-related off-target complications, ultimately improving long-term organ transplant outcomes.

In one aspect, the disclosure provides a method of reducing transplant rejection in a subject undergoing an immunosuppressant therapy, comprising administering to the subject an effective amount of pro-tolerogenic microbes that attenuate a dysbiotic state in the subject's gut microbiota.

In one aspect, the disclosure provides a method of reducing transplant rejection in a subject undergoing an immunosuppressant therapy, comprising administering to the subject an effective amount of a metabolomic composition that attenuates a dysbiotic state in the subject's gut microbiota.

In one aspect, the disclosure provides a method of reducing transplant rejection in a subject undergoing an immunosuppressant therapy, comprising
i) detecting a dysbiotic state in the subject's gut microbiota resulting from the immunosuppressant therapy; and
ii administering to the subject a therapy that attenuates the dysbiotic state in the subject's gut microbiota.

In another aspect, the disclosure provides a method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of pro-tolerogenic microbes that attenuate the dysbiotic state.

In another aspect, the disclosure provides a method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of a metabolomic composition that attenuates the dysbiotic state.

In another aspect, the disclosure provides a method of reducing transplant rejection in a subject undergoing an immunosuppressant therapy, comprising
i) detecting a dysbiotic state in the subject's gut microbiota resulting from the immunosuppressant therapy; and
ii) stopping the immunosuppressant therapy in the subject and administering to the subject a different immunosuppressant therapy.

In another aspect, the invention provides a method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of a C-C motif chemokine receptor 2 antagonist.

In another aspect, the disclosure provides a method of screening for microbial strains associated with improved transplant outcomes, comprising isolating and characterizing gut microbiota from transplant recipients with a tolerogenic immune regulatory profile, and identifying strains that promote regulatory T cell induction and suppress effector T cell activation.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Impact of immunosuppressant treatment on gut microbiome composition and function. Pairwise Bray-Curtis distance analysis comparing drug-treated groups to untreated controls and between different treatment groups to measure changes in A) taxonomic composition, and B) functional pathway abundance. Distances represent mean values per group. Untreated mice as control group. C) Principal Components Analysis of gut microbiome composition based on Bray-Curtis distance. Robust centered log ratio transformation applied on count compositional data. D) Differentially abundant taxonomic groups identified by pairwise comparisons between treatment groups and controls. Changes in abundance are expressed as log 2 fold changes relative to control group.

FIG. 2. Immunosuppressants temporally shift intestinal immune responses. A) Bar plots showing the number of differentially expressed genes (DEGs) after 7 and 30 days of treatment compared to the control group. Yellow and blue bars indicate the number of upregulated genes, while red and green bars represent the number of downregulated genes. B) Heatmap DEGs in intestinal tissue following treatment with five immunosuppressant drugs at days 7 and 30. Transcriptomic data from rapamycin-treated mice from our previously study 31 integrated in current analysis. Genes defined as differentially expressed in at least four of the five drug treatment groups at either time point. Hierarchical clustering performed on both genes (rows) and samples (columns) using Euclidean distance and ward linkage. Color scale indicates normalized Z-scores of expression values, with red denoting upregulation and blue denoting downregulation relative to no-treatment controls. C) Functional enrichment analysis of conserved intestinal transcriptional responses to immunosuppressant treatments. Dot plot showing over-represented Gene Ontology (GO) biological process pathway based on DEGs at days 7 and 30. EnrichGO pathways containing more than three genes were included in this plot. Dot size denotes the normalized enrichment score. Blue downregulated and red upregulated pathways; color shaded based on the -log ₁₀ (p-value) of enrichment. Abbr: TAC: tacrolimus, MMF: mycophenolate mofetil, PRED: prednisone, FTY: fingolimod, RAPA: rapamycin.

FIG. 3. Effects of TAC, PRED, MMF, and FTY on mLN cell content, cell distribution, and structure. Flow cytometry analysis of A) B220+ B cells and CD4+ T cells on day 30 after PRED treatment and B) Foxp3+ Tregs on day 30 after FTY treatment in mLNs. C) Heatmaps depict changes in expression with TAC, PRED, MMF, and FTY relative to controls in mLNs after 3, 7, and 30 days of treatment. IHC of D) CR and E) HEV Foxp3+Tregs on days 3, 7, and 30 in mLNs. IHC of F) CR and G) HEV La4:La5 on days 3, 7, and 30 in mLNs. Barplot showing detailed plots at each timepoint are in FIG. 13. Heatmaps depict changes in expression with TAC, PRED, MMF, and FTY relative to controls in H) CR and I) around HEV in mLNs. 3 mice/group. 2-3 LNs/mouse, 2-3 sections/LN, 7-30 fields/tissue. One-way ANOVA. * p<0.05; ** p<0.01, *** p<0.001, **** p<0.0001.

FIG. 4. Effects of TAC, PRED, MMF, and FTY on pLN cell content, cell distribution, and structure. Flow cytometry analysis of A) CD4+ T cells on day 30 after MMF treatment, and B) B220+ B cells and CD4+ T cells on day 7 and C) CD4+ T cells and Foxp3+Tregs after FTY treatment in pLNs. D) Heatmaps depict changes in expression with TAC, PRED, MMF, and FTY relative to controls in pLNs after 3, 7, and 30 days of treatment. IHC of E) CR and F) HEV Foxp3+ Tregs on days 3, 7, and 30 in pLNs. IHC of G) CR and H) La4:La5 on days 3, 7, and 30 in pLNs. Barplot showing detailed plots at each timepoint are in FIG. 14. Heatmaps depict changes in expression with TAC, PRED, MMF, and FTY relative to controls in I) CR and J) around HEV in pLNs. 3 mice/group. 2-3 LNs/mouse, 2-3 sections/LN, 7-30 fields/tissue. One-way ANOVA. * p<0.05; ** p<0.01, *** p<0.001, **** p<0.0001.

FIG. 5. MMF and FTY induce a rapid anti-inflammatory response during allogeneic stimulation. IHC for Foxp3+ Tregs in the A) PLN and C) mLN. La4:La5 in B) pLN and D) mLN. Heatmap summarizes significant changes in expression comparing allogeneic stimulation plus MMF or FTY to allogeneic stimulation alone in E) pLNs and F) mLNs. 3 mice/group. 2-3 LNs/mouse, 2-3 sections/LN, 7-30 fields/tissue. One-way ANOVA: * p<0.05, ** p<0.01, *** p<0.001, **** p<0.0001.

FIG. 6. MMF and FTY decrease the La4:La5 in FRC-Lama5-KO mLNs and pLNs. IHC showing A, D, G, J) laminin α4; B, E, H, K) laminin α5; and C, F, I, L) La4:La5 in mLNs (A-F, M) and pLNs (G-L, N) of WT and FRC-Lama4-KO (A-C and G-I) and FRC-Lama5-KO mice (D-F and J-L) on day 3. M, N) Heatmap summarizes significant changes relative to controls in M) mLN and N) PLN. 3-5 mice/group. 2-3 LNs/mouse, 2-3 sections/LN, 7-30 fields/tissue. One-way ANOVA: * p<0.05, ** p<0.01, *** p<0.001, **: * p<0.0001.

FIG. 7. Illustration of shared intestinal responses in response to immunosuppressant regimens. Created in BioRender. Ma, B. (2025) https://BioRender.com/ctcfgx9.

FIG. 8. Canonical Correspondence Analysis (CCA) to demonstrate de novo clustering of gut microbiome in A) taxonomic groups and B) microbial functional pathways based on Bray-Curtis distance. Taxonomic composition of the microbiomes using Kraken2 (v.2020.12) and Braken (v. 2.5.0) and the comprehensive mouse gut metagenome catalog (CMGM). Functional pathways characterized using HUMAN3 (Human Microbiome Project Unified Metabolic Analysis Network) (v3.0.0.alpha.3) and Uniref90 database.

FIG. 9. Immunosuppressants induce distinct alterations in DC and Treg populations in the small intestine. A) CD11c+ DCs and B) Tregs after 3, 7, and 30 days of drug treatment. C) Heatmap summarizes significant changes in DCs and Tregs compared to controls without drug treatment. Red increase, white no change, blue decrease. 3 mice/group, 3 pieces of intestine, 2-3 sections/intestine, 7-30 fields/tissue. One-way ANOVA: * p<0.05, ** p<0.01, *** p<0.001, **** p<0.0001.

FIG. 10. Analysis of differentially expressed genes (DEGs) between days 7 and 30 for each individual immune suppressants of A) TAC, B) PRED, C) MMF, and D) FTY720 using UpSet plots. Detailed list of genes is listed in Tables 1-4. Horizontal bars (left) indicate total DEG numbers, vertical bars show the size of unique and overlapping gene sets, and dots below indicate the specific conditions for each intersection, and connected dots mean those sets are shared. Arrows indicate up- or down-regulation of DEGs.

FIG. 11. Analysis of differentially expressed genes (DEGs) within and across immunosuppressant treatments. UpSet plots showing intersections of DEGs for each treatment compared to control, including A) upregulated and B) downregulated DEGs. Detailed list of genes are listed in Tables 1-4. Horizontal bars (left) indicate total DEG numbers, vertical bars show the size of unique and overlapping gene sets, dots below indicate the specific conditions for each intersection, and connected dots mean those sets are shared. Arrows indicate up- or down-regulation of DEGs. C) Functional enrichment analysis of intestinal transcriptional responses to immunosuppressant treatments. Dot plot illustrating over-represented immune-related functional pathways of DEGs in small intestinal tissues across different treatments compared to the control group, analyzed at 7 and 30 days of treatment. Gene Ontology pathways used. Dot size represents the -log 10 (p-value), indicating the statistical significance of pathway enrichment.

FIG. 12. Gut luminal metabolites after immune suppressant drug treatment. A) PLS-DA clusters by treatment groups. Circles indicate 95% confidence region. B) Metabolites contributing to separation of each treatment group in PLS-DA, ranked by variable importance in projection (VIP). C) Hierarchical clustering heatmap of metabolites after immune suppressant drug treatment. Specimens collected after treatment for 7 days, compared to no drug control. Top 50 features shown. Color bar indicates the scaled z-score of each feature. Ward linkage clustering based on Euclidian distance.

FIG. 13. Immunosuppressants distinctively influence gut metabolism. Volcano plots combine results from fold change (FC) analysis to show significantly increased metabolites after 7 days for A) TAC, B) PRED, C) MMF, or D) FTY treatment compared to no treatment control. A metabolite is shown if FC is >2 and p value is <0.05 based on 2-sample t-tests. Original metabolite measurement without normalization in FC analysis.

FIG. 14. Effects of TAC, PRED, MMF, and FTY720 on mLN cell content, cell distribution, and structure. IHC of A) CR and B) HEV Foxp3+ Tregs on days 3, 7, and 30 of drug treatment. IHC of C) CR and D) HEV La4:La5 on days 3, 7, and 30 of drug treatment. 3 mice/group 2-3 LNs/mouse, 2-3 sections/LN, 7-30 fields/tissue One-way ANOVA. * p<0.05; ** p<0.01, *** p<0.001, **** p<0.0001.

FIG. 15. Effects of TAC, PRED, MMF, and FTY720 on pLN cell content, cell distribution, and structure. IHC of A) CR and B) HEV Foxp3+ Tregs on days 3, 7, and 30 of drug treatment. IHC of C) CR and D) HEV La4:La5 on days 3, 7, and 30 of drug treatment. 3 mice/group 2-3 LNs/mouse, 2-3 sections/LN, 7-30 fields/tissue One-way ANOVA. * p<0.05; ** p<0.01, *** p<0.001, **** p<0.0001.

FIG. 16. A) Gut microbiome longitudinal changes a cardiac transplant model in which tacrolimus is administered daily after allografting. B) Mapping showing changes in various commensal families and genera. C) Chart showing changes in various commensal families and genera. D) Longitudinal profiling of the ileal intraluminal metabolome in the cardiac allograft model receiving daily tacrolimus.

FIG. 17. A) Experimental scheme for treating mice with antibiotics on days −7 to −1, then on day 0 cardiac transplantation plus fecal microbiota transfer (FMT) sourced from individual strains of bacteria, and then sampling groups of mice on days 0-40.

FIG. 18. A) Trajectory analysis of Bifido and and Desulfo FMT groups using PCoA connected by temporal vectors. B) Mixed-effects modeling showing that, comparing to Bifido group, tacrolimus-only controls were significantly enriched for Muribaculaceae, including Muribaculum, CAG-485, Paramuribaculum, and Duncaniella, as well as Erysipelatoclostridium.

FIG. 19. A) Trajectory analysis using PCoA connected by temporal vectors. B) PCoA plots at each time point, where Bifido-treated mice clustered apart from the other groups. C) Species-level heatmaps ordered by time and group, showing coordinated, time-structured responses to Bifido gavage. D) Muribaculaceae lineages, which were enriched in the PBS and Desulfo groups, expanded in a reciprocal manner.

FIG. 20. A) Immunohistochemistry of cardiac grafts, treated as in FIG. 17A, harvested on day 3 or day 40 after transplant, and sections of the cardiac grafts stained for hematoxylin and eosin (H&E) for inflammatory cell infiltrates or with trichrome for fibrosis. The staining was then quantified for H&E, trichrome, and a composite combined score for rejection. Panels show representative images of the histologic sections and quantification of the scores. B) Same grafts as in A that were assessed for infiltration of blood vessels by lymphocytes as a measure of vascular rejection.

FIG. 21. A) Experimental scheme for treating mice, similar to FIG. 17, and FMT sourced from human cardiac transplant recipients with (CAV) or without (NCAV) cardiac allograft vasculopathy or from a healthy nontransplant human fecal donor.

FIG. 22. A) Fibrosis scores in cardiac allografts. B) CD4⁺ and cells in in cardiac allografts. C) Laminin α4:α5 ratios in peripheral lymph nodes. D) and E) Trichrome staining FMT recipients. Assessment of graft blood vessels revealed severe vasculitis in the CAV FMT group, characterized by lymphocytic infiltrates inside or surrounding vessels, a key clinical criterion for grading cardiac graft rejection.

FIG. 23. C57BL/6 recipients given abx day −6 to −1; day 0 BALB/c heart Txp and FMT; and tacrolimus 3 mg/kg/d days 0-60. n=9-10 mice/gp. A) Graft inflammation on d60 with Bifido or Desulfo. Photomicrographs of H&E and trichrome-stained graft sections of B) WT stool FMT, C) Bifido, and D) Desulfo gavage. Scale bar: 200 μm. Unpaired Student's t test.

FIG. 24. C57BL/6 recipients given abx day −6 to −1, BALB/c heart Txp and bacterial FMT on day 0, and tacrolimus 3 mg/kg/d for 40 days. 2 separate experiments with 3-5 mice/gp/expt. A) CD4 and B) CD8 T cells in the cardiac grafts. LN laminin!4:15 ratio in C) CR and D) HEV in peripheral LNs (pLNs). MFI: mean fluorescence intensity normalized by sum of mean. One-way ANOVA with Tukey's multiple comparison.

FIG. 25. C57BL/6 mice given tacrolimus (3 mg/kg/d), rapamycin (5 mg/kg/d), mycophenolate mofetil (MMF, 30 mg/kg/day), or fingolimod (FTY, 3 mg/kg/day). Control received PBS. n=3 mice/gp. A) Pairwise Bray-Curtis distance analysis comparing immunosuppressant drug-treated groups to untreated controls. B) Differentially abundant taxonomic groups identified by pairwise comparisons between treatment groups and controls. Changes in abundance as log 2 fold changes relative to control. C) Gut intraluminal and serum metabolome using target choline and amino acid metabolism panel by ultra-high pressure liquid chromatography and triple quadrupole mass spectrometers12-14.

FIG. 26. Longitudinal profiling gut microbiome after Bifido or Desulfo FMT. A) C57BL/6 recipients given abx day −6 to −1, cardiac Txp and bacterial FMT on day 0, tacrolimus (3 mg/kg/d) for 40 days. Controls received PBS. n=3 mice/gp. B) graft blood vessels vascular score, assessed using cardiac graft rejection grading scale based on severity of lymphocyte infiltrate inside or surrounding vessels4. ~10 fields/section, score averaged among all fields. Scale bar: 100 μm. Unpaired Student's t tests. C) Non-metric multidimensional scaling plots using Bray-Curtis distance. D) Cladogram of logarithmic linear discriminant analysis effect size (LEfSe)⁴⁰ showing microbial biomarkers using pairwise non-parametric Kruskal-Wallis test.

FIG. 27. C57BL/6 recipients given abx day −6 to −1; BALB/c heart Txp and FMT on d0; PBS as no-FMT control; tacrolimus (3 mg/kg/d) on days 0-6. n=3 mice/gp. A) Partial-least-squares discriminant analysis³⁶ of metabolome using capillary electrophoresis-mass spectrometry³⁷⁻³⁹. B) Intestinal permeability using FITC-dextran. C) Pathway topology adapted from KEGG arginine metabolism (map00330)⁴⁴. All plotted metabolites p<0.05. ADMA/SDMA: asymmetric/symmetric dimethylarginine; SAM: S-adenosylmethionine.

FIG. 28. Polyamines and Treg Foxp3 expression. A) Treg metabolome showed higher levels of polyamines than exTregs. CD25hiFoxp3 hi Tregs migrated across LEC to CCL19 in Boyden chambers. Tregs harvested from upper and lower chambers after 16 hrs and sorted for those that remained CD25^{hi}Foxp^{hi} Tregs and those converted to CD25^{lo}Foxp3^{lo} exTregs. Metabolome assessed by CE-MS. Conc. as pmol/106 cells. B) SAM (250 μM) inhibited Treg induction. Treg induction with 10⁵ naïve CD4 T cells plated with anti-CD3 (5 μg/mL), IL-2 (20 ng/mL), TGFb (5 μg/mL), anti-CD28 (1 μg/mL), and SAM for 3 days. Cells stained with anti-CD4, anti-CD25, and intracellularly stained with anti-Foxp3-APC and assessed by FACS.

FIG. 29. Gut microbiome of human cardiac transplant patients (10 CAV, 6 non-CAV). A) PCoA; B) diversity using species richness; C) heatmap of top 15 species. Taxonomy using MetaPhlAn v41. Z-score scaled by row.

FIG. 30. C57BL/6 recipients given abx day −6 to −1; BALB/c heart Txp day 0; human donor FMT day 0, 7, 14, 21; tacrolimus 3 mg/kg/d for 60 days. n=9-10 mice/gp. Control using a healthy donor. A) Graft fibrosis d60; B) Flow cytometry for CD4+ and CD8+ T cells in grafts; C) pLNlaminin α4:α5 ratio. H&E and trichrome of D) graft sections and E) graft blood vessels. Scale bar: 100 μm. Student's t tests.

FIG. 31. Study design. I) C57BL/6 (H-2^{b}) mice given abx for 6 days to deplete endogenous microbiota³⁵. After 24 hrs without abx, Txp with BALB/c (H-2^{b}) hearts and bacteria FMT via gavage on D0, and immunosuppression with tacrolimus (3 mg/kg/d s.c.) D0 to D60. FITC-dextran measurement, intestine, blood, hearts, spleens and LNs collected at each harvest. II) Bifido daily for 7 days (10⁷ cfu/day) before Txp. III) Bifido daily for 7 days after Txp, then weekly till harvest. Specimens at euthanasia: intestine, mesenteric and peripheral LNs, spleen, heart, intestinal contents from scraping intestinal mucosa, and sera. FITC-dextran gavage prior to euthanasia to measure intestinal permeability.

FIG. 32. Mouse heart snRNASeq (n=9).

### DETAILED DESCRIPTION

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

In one embodiment, the disclosure provides a method of reducing transplant rejection in a subject undergoing an immunosuppressant therapy, comprising administering to the subject an effective amount of pro-tolerogenic microbes that attenuate a dysbiotic state in the subject's gut microbiota.

In another embodiment, the disclosure provides a method of reducing transplant rejection in a subject undergoing an immunosuppressant therapy, comprising administering to the subject an effective amount of a metabolomic composition that attenuates a dysbiotic state in the subject's gut microbiota.

In another embodiment, the disclosure provides a method of reducing transplant rejection in a subject undergoing an immunosuppressant therapy, comprising
i) detecting a dysbiotic state in the subject's gut microbiota resulting from the immunosuppressant therapy; and
ii) administering to the subject a therapy that attenuates the dysbiotic state in the subject's gut microbiota.

In another embodiment, the disclosure provides a method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of pro-tolerogenic microbes that attenuate the dysbiotic state.

In another embodiment, the disclosure provides a method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of a metabolomic composition that attenuates the dysbiotic state.

In another embodiment, the disclosure provides a method of reducing transplant rejection in a subject undergoing an immunosuppressant therapy, comprising
i) detecting a dysbiotic state in the subject's gut microbiota resulting from the immunosuppressant therapy; and
ii) stopping the immunosuppressant therapy in the subject and administering to the subject a different immunosuppressant therapy.

In another embodiment, the invention provides a method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of a C-C motif chemokine receptor 2 antagonist.

The terms “effective amount” or “therapeutically effective amount” or “therapeutic effect” refer to an amount of an agent or composition described herein, or other drug effective to achieve a desired outcome, such as attenuating a dysbiotic state in a subject undergoing immunosuppressant therapy.

In some embodiments, the C-C motif chemokine receptor 2 antagonist is selected from RS504393, CCX140-B, INCB3284/INCB3344, BMS-681, CCR2-RA-[R], propagermanium, CAS 445479-97-0 and Kaempferol 3-(2,4-di-E-p-coumaroylrhamnoside. In some embodiments, the antagonist is administered in an amount of from about 0.5 2 mg/kg/d to 5 mg/kg/day.

In another embodiment, the disclosure provides a method of screening for microbial strains associated with improved transplant outcomes, comprising isolating and characterizing gut microbiota from a transplant recipient with a tolerogenic immune regulatory profile, and identifying strains that promote regulatory T cell induction and suppress effector T cell activation.

In some embodiments, the therapy that attenuates the dysbiotic state in the subject's gut microbiota (e.g., pro-tolerogenic microbes or metabolomic composition) is administered prior to the subject undergoing a transplant, e.g., in some embodiments where detecting the dysbiotic state is optional or not required. In some embodiments, the therapy is administered following the transplant, e.g., from 1-7, 1-14, 1-21, 1-30, 1-40, 1-50, 1-60, 1-80, or from 1-90 days or more following transplant.

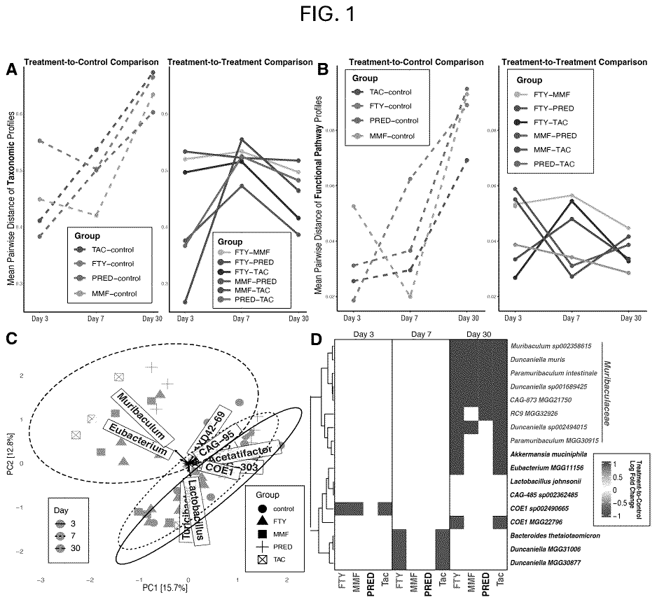

Methods for detecting the dysbiotic state are not necessarily limiting. In some embodiments, the methods comprise analyzing the gut microbiota of the subject and detecting the dysbiotic state. In some embodiments, the subject's gut microbiota is analyzed for an increase, relative increase, decrease, or relative decrease in the presence of one or more particular microbes, for example, as shown in FIG. 1D. In some embodiments, the subject's gut microbiota is analyzed for reduced microbial diversity.

In some embodiments, the subject's gut microbiota is analyzed for an increase or relative increase in the presence of one or more pathobionts. In some embodiments, the pathobiont is selected from the group consisting of adherent-invasive Escherichia coli (AIEC), Bacteroides vulgatus, Bacteroides fragilis (especially enterotoxigenic), Enterococcus faecalis, Fusobacterium nucleatum, Clostridioides difficile, Erysipelotrichaceae and Muribaculaceae.

In some embodiments, the subject's gut microbiota is analyzed for an increase, or relative increase in the presence of Parabacteroides distasonis. In some embodiments, the subject's gut microbiota is analyzed for an increase, or relative increase in the presence of Muribaculaceae (formerly known as S24-7), including Duncaniella, Paramuribaculum intestinale, and CAG-873. In some embodiments, the subject's gut microbiota is analyzed for an increase, or relative increase in the presence of Erysipelatoclostridium.

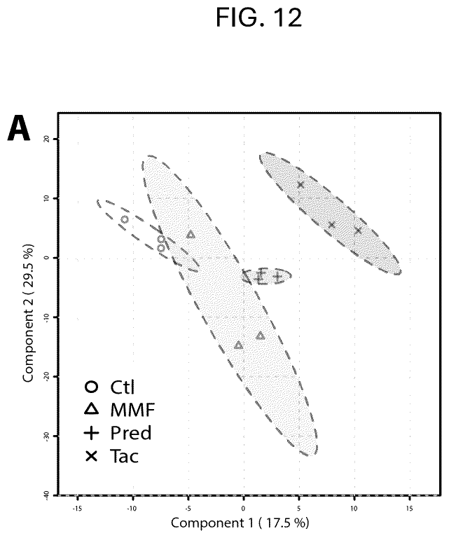

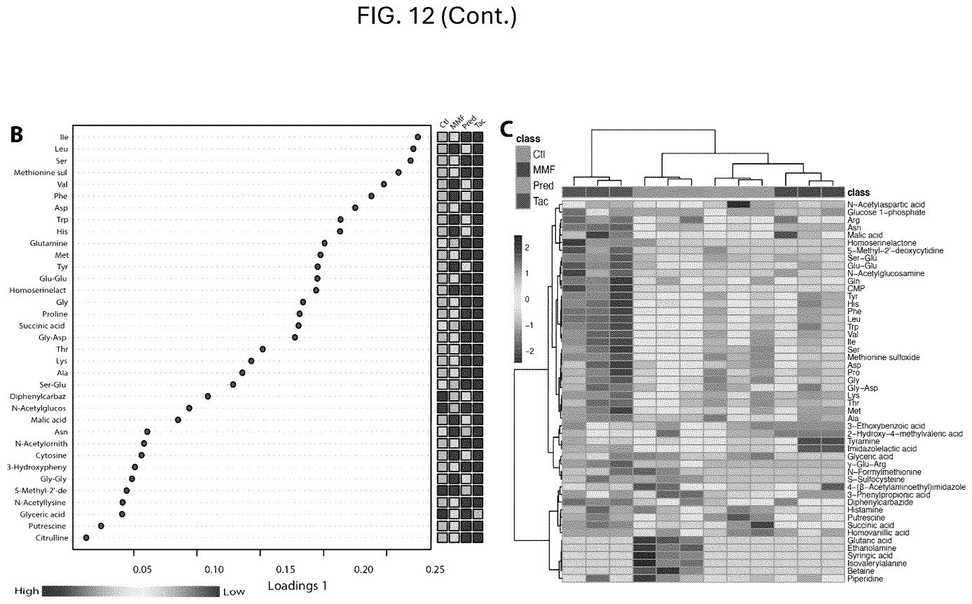

In some embodiments, detecting the dysbiotic state comprises analyzing the subject's metabolome profile. In some embodiments, the detecting comprises detecting changes in the presence, absence or relative amounts of one or more metabolites that indicate that the subject is in a dysbiotic state. In some embodiments, the one or more metabolites are selected from the metabolites shown in FIG. 12C.

In some embodiments, the detecting comprises detecting a change in the presence, absence or relative amounts in one or more microbiota-derived aromatic and aromatic and choline-axis metabolites, 3-(3,4-dihydroxyphenyl) propionic acid (DHPPA), 3-(4-hydroxyphenyl) propionic acid (HPPA), 3-(4-hydroxyphenyl) lactate, hydrocaffeic acid, phenylacetylglutamine (PAGIn), trimethylamine N-oxide (TMAO), succinic acid, glycerophosphocholine, phosphocholine, glutaric acid, 4PY (N-methyl-4-pyridone-3-carboxamide), and Ne-(1-carboxyethyl)-L-lysine (AGE).

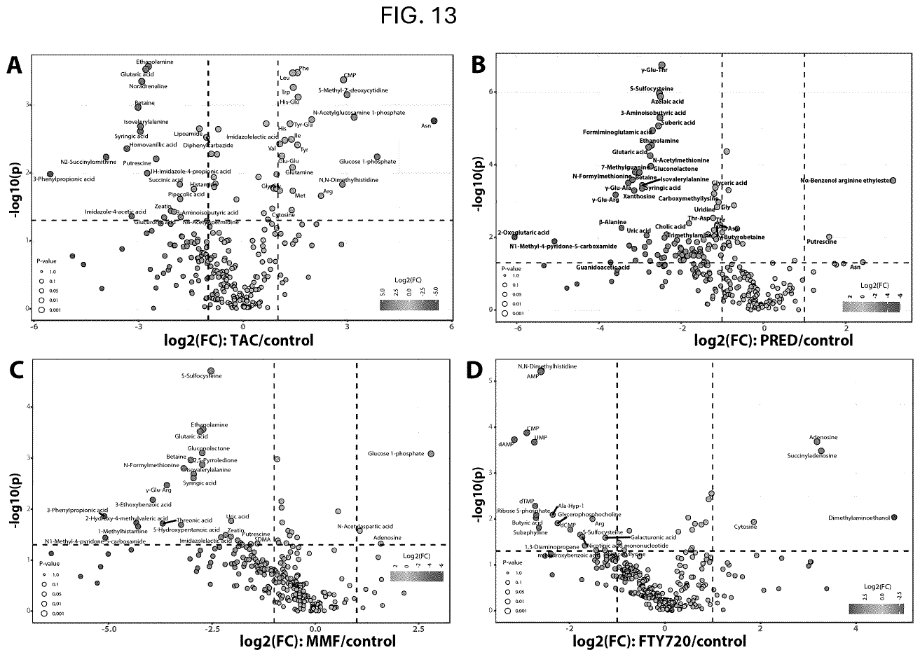

In some embodiments, detecting the dysbiotic state comprises detecting a change in the presence, absence or relative amounts of one or more metabolites shown in FIG. 13A. In some embodiments, the subject is undergoing immunsuppressive therapy with tacrolimus and exhibits a change in the presence, absence or relative amounts of one or more metabolites shown in FIG. 13A.

In some embodiments, detecting the dysbiotic state comprises detecting a change in the presence, absence or relative amounts of one or more metabolites shown in FIG. 13B. In some embodiments, the subject is undergoing immunsuppressive therapy with prednisolone and exhibits a change in the presence, absence or relative amounts of one or more metabolites shown in FIG. 13B.

In some embodiments, detecting the dysbiotic state comprises detecting a change in the presence, absence or relative amounts of one or more metabolites shown in FIG. 13C. In some embodiments, the subject is undergoing immunsuppressive therapy with mycophenolate mofetil and exhibits a change in the presence, absence or relative amounts of one or more metabolites shown in FIG. 13C.

In some embodiments, detecting the dysbiotic state comprises detecting a change in the presence, absence or relative amounts of one or more metabolites shown in FIG. 13D. In some embodiments, the subject is undergoing immunsuppressive therapy with fingolimod and exhibits a change in the presence, absence or relative amounts of one or more metabolites shown in FIG. 13D.

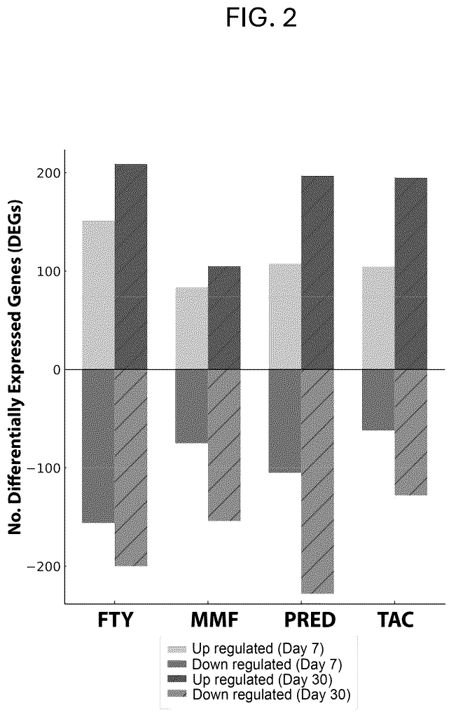

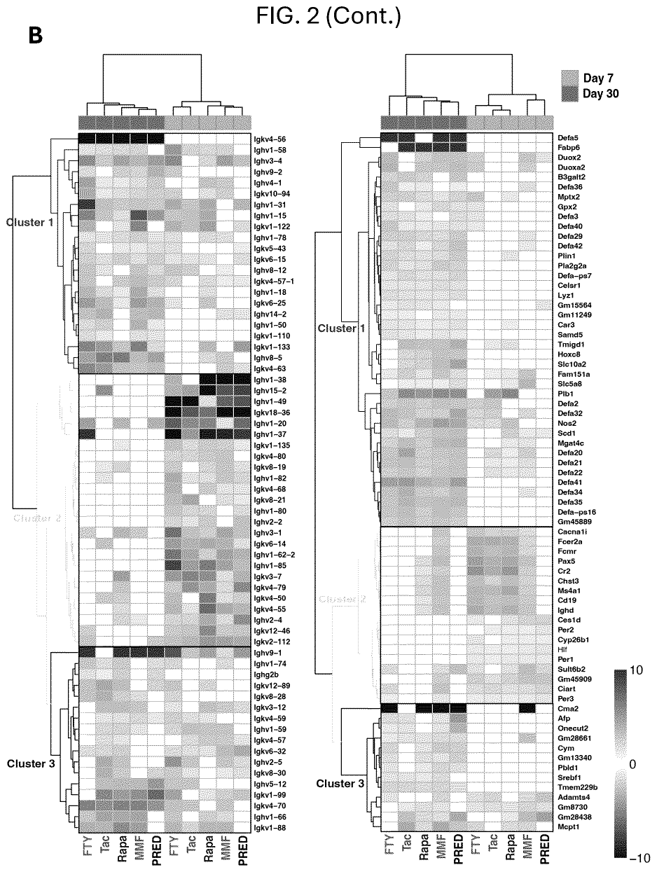

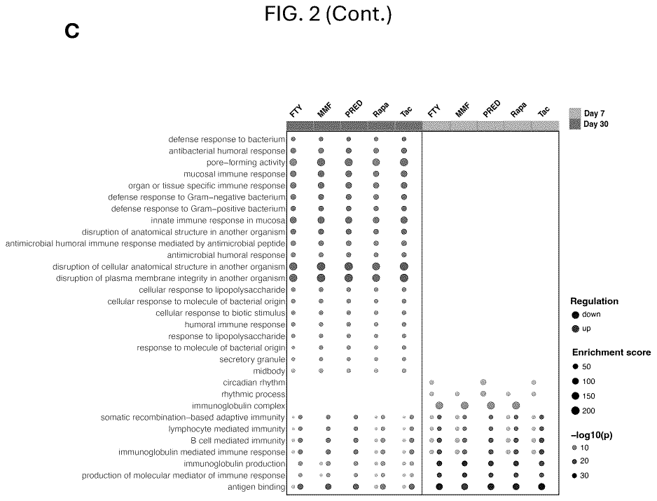

In some embodiments, detecting the dysbiotic state comprises detecting expression of one or more differentially expressed genes resulting from the immunosuppressive therapy. In some embodiments, the one or more differentially expressed genes are provided in FIG. 2B, Table 1, Table 2, Table 3 and/or Table 4. Table 1 shows genes upregulated after 7 days of treatment with FTY, MMF, Pred, Rapa or Tac. Table 2 shows genes upregulated after 30 days of treatment with FTY, MMF, Pred, Rapa or Tac. Table 3 shows genes downregulated after 7 days of treatment with FTY, MMF, Pred, Rapa or Tac. Table 4 shows genes downregulated after 30 days of treatment with FTY, MMF, Pred, Rapa or Tac. In some embodiments, the differentially expressed gene is upregulated by the immunosuppressive therapy. In some embodiments, the differentially expressed gene is downregulated by the immunosuppressive therapy. In some embodiments, the downregulation or upregulation occurs after about 7 days or after about 30 days following immunosuppressive therapy.

In some embodiments, the one or more differentially expressed genes are selected from α-defensins (Defa-ps16, Defa-ps7, Defa2, Defa20-22, Defa29, Defa3, Defa32, Defa34-36, Defa40-42, Defa5), lysozyme (Lyz1), secretory phospholipase A2 group IIA (Pla2g2a), oxidase genes (Duox2, Duoxa2), inducible nitric oxide synthase (iNOS, Nos2), glutathione peroxidase (Gpx2), Ciart, Per1, Per2, Per3, Hlf, Cyp26b1, Cesld, Sult6b2, Cd19, Fcer2a, Pax5, Onecut2, Tmem229b, Srebf1, Chst3, Adamts4, Mcpt1 and Cma2.

In some embodiments, detecting the dysbiotic state comprises detecting one or more conditions in the subject. In some embodiments, detecting the dysbiotic state comprises analyzing the lymph nodes in the subject, and detecting a pro-inflammatory environment in the lymph nodes, e.g., characterized by reduced Treg presence and/or decreased laminin α4:α5 ratios. In some embodiments, a ratio of regulatory T cells (Tregs) to effector T cells is analyzed. In some embodiments, mesenteric lymph nodes are analyzed. In some embodiments, peripheral lymph nodes are analyzed.

In some embodiments, laminin α4:α5 ratios are assayed within the T cell cortex of the cortical ridge (CR) and around high endothelial venules (HEVs) in the lymph nodes.

In some embodiments, the subject is undergoing an immunosuppressive therapy with one or more immunosuppressive agents selected from a calcineurin inhibitor, a mTOR inhibitor, a glucocorticoid or steroid, an inosine monophosphate dehydrogenase (IMPDH) inhibitor, an anti-metabolite and a sphingosine-1-phosphate (SIP) receptor antagonist. In some embodiments, a combination of these agents are administered as an immunosuppressive therapy.

In some embodiments, the subject is undergoing immunosuppressive therapy with one or more immunosuppressive agent(s) selected from tacrolimus, mycophenolate mofetil, mycophenolic acid, azathioprine, prednisone, fingolimod, rapamycin, cyclosporine, sirolimus, everolimus, belatacept, campath, anti-IL-2, anti-CD20, anti-CD38, complement inhibitors, anti-thymocyte globulin, anti-CD40, and anti-CD40L.

In some embodiments, the subject is undergoing a regimen comprising tacrolimus, mycophenolate and prednisone.

In some embodiments, the dysbiotic state is detected about 7 days, about 10 days, about 15 days, about 20 days, about 25 days, about 30 days, about 35 days, about 40 days, about 45 days, about 50 days, about 60 days, about 70 days, about 80 days, or about 90 days or more following initiation of immunosuppressive therapy.

The subject to be treated is not limiting. The term “subject” refers to any animal (e.g., a mammal), including, but not limited to humans, non-human primates, rodents, pets, such as dogs, cats, horses, etc., agricultural animals such as cows, sheep, goats and the like, which is to be the recipient of a particular treatment. Typically, the terms “subject” and “patient” are used interchangeably herein in reference to a human subject.

The organ or tissue that is to be transplanted is not limiting. In some embodiments, the transplant is a cardiac allograft transplant. In some embodiments, the method reduces chronic cardiac allograft vasculopathy (CAV). In some embodiments, the transplant is a kidney transplant. In some embodiments, the tissue that is transplanted is skin. In some embodiments, the transplant is selected from heart, kidney, skin, liver, lung, bone marrow, tendon, cornea, pancreas, ligament, heart valve, bone, and vascularized composite allografts, such as hands, arms, legs, and faces. The source of the transplanted organ or tissue is not limiting. In some embodiments, it is from the same species, e.g., human, as the recipient. In some embodiments, it is from a different recipient. For example, in some embodiments, the donor is a mammal such as a pig, and the recipient is a mammal such as a human.

In some embodiments, the method modulates gut microbiota and metabolic activities to enhance regulatory T cell induction and suppress effector T cell activation, or increase the ratios of regulatory T cells to effector T cells. In some embodiments, the method restores gut microbiota balance and regulates immune responses. In some embodiments, the method alters laminin α4:α5 ratios in lymph nodes to promote immune tolerance. In some embodiments, the method restores amino acid metabolism, short-chain fatty acid production, and polyamine synthesis.

In some embodiments, the method reduces abundance of pathobionts in the gut, such as Muribaculaceae and Erysipelotrichaceae, restores short chain fatty acids (SCFAs) producers, and expands Tregs.

In some embodiments, the method reduces vascular inflammation and promotes graft survival. In some embodiments, the method reduces graft inflammation and fibrosis.

Effective amounts of pro-tolerogenic microbes can help to attenuate the dysbiotic state in the subject and thereby provide for a more tolerant immune environment in the subject. The pro-tolerogenic microbes that can be administered are not particularly limiting. In some embodiments, the pro-tolerogenic microbes comprise one or more of Akkermansiaceae (such as Akkermansia muciniphila) Bacteroidaceae (such as Bacteroides thetaiotaomicron), Enterobacteriaceae, Clostridia (cluster XIVa and IV), Lactobacilluseae, Lachnospiraceae, Eubacterium, Bifidobacterium, CAG-180 (Oscillospirales), UBA2882, Bacteroides, Prevotella, Ruminiclostridium (Ruminococcaceae), and COE1 and 1XD8-76. In some embodiments, the Bifidobacterium is B. longum. In some embodiments, the Bifidobacterium is B. pseudolongum. In some embodiments, the subspecies is globosum.

In some embodiments, the pro-tolerogenic microbes can comprise a fecal microbiota transfer (FMT). In some embodiments, the FMT is sourced from one or more human transplant recipients with pro-tolerogenic immune regulatory profiles, wherein the FMT induces metabolic and immune tolerance changes that promote graft survival.

In some embodiments, the FMT comprises one or more microbes selected from Bacteroides uniformis, Bacteroides vulgatus, Parabacteroides, Blautia, Butyricimonas, Anaerostipes, and/or Hungatella. In some embodiments, the FMT comprises Ruminococcus, Prevotella, Eubacterium, and UBA-7182 (Lachnospiraceae).

In some embodiments, the subject is administered an effective amount of a metabolomic composition. In some embodiments, the composition comprises an effective amount of polyamines, such as spermidine and/or spermine. In some embodiments, the composition comprises one or more of polyamines, short-chain fatty acids, secondary bile acids or a combination thereof. In some embodiments, the composition comprises one or more of adenosine, spermidine, butyrate, indole-AhR ligands, NAD+ boosting agents and a combination thereof. In some embodiments, the metabolomic composition is derived from gut microbiota. In some embodiments, the composition reconditions the intestinal niche and/or has protolerogenic effects.

The therapy or therapeutic composition can be administered one time or more than one time, for example, more than once per day, daily, weekly, monthly, or annually. The duration of treatment is not limiting. The duration of administration of the therapeutic agent can vary for each individual to be treated/administered depending on the individual cases. In some embodiments, the therapy or therapeutic composition can be administered continuously for a period of several days, weeks, months, or years of treatment or can be intermittently administered where the individual is administered the therapy or therapeutic composition for a period of time, followed by a period of time where they are not treated, and then a period of time where treatment resumes as needed. Throughout treatment, the state of dysbiosis in the subject can be monitored and treatment can be adjusted accordingly.

For example, in some embodiments, the individual to be treated is administered the therapy or therapeutic composition of the invention daily, every other day, every three days, every four days, 2 days per week 3 days per week, 4 days per week, 5 days per week or 7 days per week. In some embodiments, the individual is administered the therapy or therapeutic composition for 1 week, 2 weeks, 3 weeks, 4 weeks, 1 month, 2 months, 3 months, 4 months, 5 months, 6 months, 7 months, 8 months, 9 months, 10 months, 11 months, 1 year or longer.

In some embodiments, the method further comprises stopping the immunosuppressant therapy that is causing the dysbiotic state in the subject and optionally administering to the subject a different therapy, such as a different immunosuppressant therapy. In some embodiments, the different immunosuppressant therapy comprises administering an effective amount of mycophenolate mofetil (MMF), and/or fingolimod (FTY). Following a change in therapy, the subject can be further monitored, for example, for the worsening or amelioration of the dysbiotic state.

In some embodiments, the therapeutic compositions of the present disclosure include classic pharmaceutical preparations. Administration of these compositions according to the present disclosure will be via any common route so long as the target tissue is available via that route. Such routes of administration are preferably oral, nasal, buccal, rectal, or mucosal (including buccal, and sublingual). In some embodiments the routes can include, oral, intravenous, intramuscular, subcutaneous, intraperitoneal, and rectal routes.

Administration can also be via nasal spray, surgical implant, internal surgical paint, infusion pump, or via catheter, stent, balloon or other delivery devices, such as nanoparticles or microparticles. The most useful and/or beneficial mode of administration can vary.

The compositions can be administered in a variety of dosage forms. Pharmaceutical compositions suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions.

In one embodiment, compositions may be administered orally in combination with a pharmaceutically acceptable vehicle such as an inert diluent or an edible carrier. Oral compositions may be enclosed in hard or soft shell gelatin capsules, may be compressed into tablets or may be incorporated directly with the food of the patient's diet. The percentage of the composition and preparations may be varied; however, the amount of substance in such therapeutically useful compositions is preferably such that an effective dosage level will be obtained.

Hard capsules containing the compositions may be made using a physiologically degradable composition, such as gelatin. Such hard capsules comprise the active agents, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the drug, various soluble excipients, such as lactose, powdered sucrose, dextrose, and mannitol. The solid dosage forms of the present technology may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT), zein, shellac, and polysaccharides.

Powdered and granular compositions of a pharmaceutical preparation may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of pharmaceutical compositions which are suitable for oral administration may be prepared, packaged, and sold either in liquid form or in the form of a dry product intended for reconstitution with water or another suitable vehicle prior to use.

A tablet containing one or more agents described herein may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present technology may also be sustained release (i.e., releasing the active agents over a prolonged period of time), and may or may not also be delayed release. Sustained release compositions are known in the art and are generally prepared by dispersing the active agent(s) within a matrix of a gradually degradable or hydrolyzable material, such as an insoluble plastic, a hydrophilic polymer, or a fatty compound. Alternatively, a solid dosage form may be coated with such a material.

In another aspect, the invention provides a method of screening for microbial strains associated with improved transplant outcomes, comprising isolating and characterizing gut microbiota from transplant recipients with a tolerogenic immune regulatory profile, and identifying strains that promote regulatory T cell induction and suppress effector T cell activation.

The present invention also includes kits useful in performing assays for detecting a dysbiotic state in the subject's gut microbiota of the present disclosure. Kits of the disclosure include a suitable container comprising one or more detection reagents, which can include various primers to detect differentially expressed genes, or other reagents to detect increased or decreased levels of metabolites, or the various microbes. Control samples and/or instructions are also included.

### Sample Embodiments

This section describes exemplary compositions and methods of the invention, presented without limitation, as a series of paragraphs, some or all of which may be alphanumerically designated for clarity and efficiency. Each of these paragraphs can be combined with one or more other paragraphs, and/or with disclosure from elsewhere in this application, including the materials incorporated by reference, in any suitable manner. Some of the paragraphs below expressly refer to and further limit other paragraphs, providing without limitation examples of some of the suitable combinations.
1. A method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of pro-tolerogenic microbes that attenuate the dysbiotic state in the subject's gut microbiota.
2. The method of paragraph 1, wherein detecting the dysbiotic state comprises analyzing the gut microbiota for an increase, relative increase, decrease, or relative decrease in one or more microbes.
3. The method of paragraph 2, wherein the subject's gut microbiota is analyzed for a relative increase in the presence of one or more pathobionts.
4. The method of paragraph 3, wherein the one or more pathobionts belong to the family of Muribaculaceae.
5. The method of any of paragraphs 1-4, wherein detecting the dysbiotic state comprises analyzing the subject's metabolome profile for an increase, relative increase, decrease, or relative decrease in one or more metabolites (for example, in FIGS. 12C, 13A, 13B, 13C, or 13D).
6. The method of any of paragraphs 1-5, wherein the detecting comprises detecting the presence, absence or relative amounts of one or more differentially expressed genes (for example, in FIG. 2C, Table 1, Table 2, Table 3 or Table 4).
7 The method of any of paragraphs 1-6, wherein the detecting comprises analyzing the lymph nodes in the subject.
8. The method of paragraph 7, wherein the lymph nodes have a pro-inflammatory architecture, characterized by reduced Treg presence and/or decreased laminin α4:α5 ratios.
9. The method of paragraph 7 or 8, wherein mesenteric lymph nodes are analyzed.
10. The method of paragraph 7 or 8, wherein peripheral lymph nodes are analyzed.
11. The method of any of paragraphs 1-10, wherein the immunosuppressive therapy comprises one or more immunosuppressive agent(s) selected from tacrolimus, mycophenolate mofetil, mycophenolic acid, azathioprine, prednisone, fingolimod, rapamycin, cyclosporine, sirolimus, everolimus, and belatacept.
12. The method of any of paragraphs 1-11, wherein the immunosuppressant therapy comprises a combination of tacrolimus, mycophenolate and prednisone.
13. The method of any of paragraphs 1-12, wherein the dysbiotic state is detected about 7 days, about 10 days, about 15 days, about 20 days, about 25 days or about 30 days, about 35 days, about 40 days, about 45 days or about 50 days or more following initiation of immunosuppressive therapy.
14. The method of any of paragraphs 1-13, wherein the transplant is a cardiac allograft transplant or kidney allograft transplant.
15. The method of paragraph 14, wherein the transplant is a cardiac allograft transplant, wherein the method reduces chronic cardiac allograft vasculopathy (CAV).
16. The method of any of paragraphs 1-15, wherein the administration of pro-tolerogenic microbes modulates gut microbiota and metabolic activities.
17. The method of any of paragraphs 1-16, wherein the administration of pro-tolerogenic microbes modulates gut microbiota and metabolic activities to enhance regulatory T cell induction and suppress effector T cell activation.
18. The method of any of paragraphs 1-17, wherein the administration restores gut microbiota balance or diversity.
19. The method of any of paragraphs 1-18, wherein the administration alters laminin α4:α5 ratios in lymph nodes to promote immune tolerance.
20. The method of any of paragraphs 1-19, wherein the administration restores amino acid metabolism, short-chain fatty acid production, and polyamine synthesis in gut microbiota.
21. The method of any of paragraphs 1-20, wherein the administration reduces vascular inflammation and promotes graft survival.
22. The method of any of paragraphs 1-21, wherein the administration reduces graft inflammation and fibrosis.
23. The method of any of paragraphs 1-22, wherein the pro-tolerogenic microbes comprise one or more of Akkermansiaceae, Bacteroidaceae, Enterobacteriaceae, Clostridia (cluster XIVa and IV), Lactobacilluseae, Lachnospiraceae, Eubacterium, Bifidobacterium, CAG-180 (Oscillospirales), UBA2882, Bacteroides, Prevotella, Ruminiclostridium (Ruminococcaceae), Bacteroides uniformis, Bacteroides vulgatus, Parabacteroides, Blautia, Butyricimonas, Anaerostipes, Hungatella, COE1, 1XD8-76, Ruminococcus and UBA-7182.
24. The method of paragraph 23, wherein the pro-tolerogenic microbes comprises Bifidobacterium, wherein the Bifidobacterium is Bifidobacterium longum or Bifidobacterium pseudolongum.
25. The method of paragraph 24, wherein the Bifidobacterium is Bifidobacterium longum.
26. The method of any of paragraphs 1-25, wherein the pro-tolerogenic microbes comprise a fecal microbiota transfer (FMT).
27. The method of paragraph 26, wherein the FMT is sourced from a human transplant recipient(s) with a tolerogenic immune regulatory profile, wherein the FMT induces metabolic and/or immune tolerance changes that promote graft survival.
28. The method of any of paragraphs 1-27, wherein the subject is further administered an effective amount of a metabolomic composition.
29. The method of paragraph 28, wherein the metabolomic composition comprises one or more of polyamines, short-chain fatty acids, and secondary bile acids.
30. The method of any of paragraphs 28 or 29, wherein the metabolomic composition is derived from gut microbiota.
31. The method of any of paragraphs 28-30, wherein the metabolomic composition comprises an effective amount of spermidine and/or spermine.
32. The method of any of paragraphs 1-31, wherein the method further comprises stopping the immunosuppressant therapy in the subject and administering to the subject a different immunosuppressant therapy.
33. The method of paragraph 32, wherein the different immunosuppressant therapy comprises administering an effective amount of mycophenolate mofetil (MMF), and/or fingolimod (FTY).
34. A method of reducing transplant rejection in a subject undergoing an immunosuppressant therapy, comprising
i) detecting a dysbiotic state in the subject's gut microbiota resulting from the immunosuppressant therapy; and
ii) administering to the subject an effective amount of a metabolomic composition that attenuates the dysbiotic state in the subject's gut microbiota.
35. The method of paragraph 34, wherein the metabolomic composition comprises one or more of polyamines, short-chain fatty acids, and secondary bile acids.
36. The method of any of paragraphs 34 or 35, wherein the metabolomic composition is derived from gut microbiota.
37. The method of any of paragraphs 34-36, wherein the metabolomic composition comprises an effective amount of spermidine and/or spermine.
38. The method of any of paragraphs 34-37, wherein detecting the dysbiotic state comprises analyzing the gut microbiota for an increase, relative increase, decrease, or relative decrease in one or more microbes.
39. The method of paragraph 38, wherein the subject's gut microbiota is analyzed for a relative increase in the presence of one or more pathobionts.
40. The method of paragraph 39, wherein the one or more pathobionts belong to the family of Muribaculaceae.
41. The method of any of paragraphs 34-40, wherein detecting the dysbiotic state comprises analyzing the subject's metabolome profile for an increase, relative increase, decrease, or relative decrease in one or more metabolites (for example, in FIGS. 12C, 13A, 13B, 13C, or 13D).
42. The method of any of paragraphs 34-41, wherein the detecting comprises detecting the presence, absence or relative amounts of one or more differentially expressed genes (for example, in FIG. 2C, Table 1, Table 2, Table 3, or Table 4).
43. The method of any of paragraphs 34-42, wherein the detecting comprises analyzing the lymph nodes in the subject.
44. The method of paragraph 43, wherein the lymph nodes have a pro-inflammatory architecture, characterized by reduced Treg presence and/or decreased laminin α4:α5 ratios.
45. The method of paragraph 43 or 44, wherein mesenteric lymph nodes are analyzed.
46. The method of paragraph 43 or 44, wherein peripheral lymph nodes are analyzed.
47. The method of any of paragraphs 34-46, wherein the immunosuppressive therapy comprises one or more immunosuppressive agent(s) selected from tacrolimus, mycophenolate mofetil, mycophenolic acid, azathioprine, prednisone, fingolimod, rapamycin, cyclosporine, sirolimus, everolimus, and belatacept.
48. The method of any of paragraphs 34-47, wherein the immunosuppressant therapy comprises a combination of tacrolimus, mycophenolate and prednisone.
49. The method of any of paragraphs 34-48, wherein the dysbiotic state is detected about 7 days, about 10 days, about 15 days, about 20 days, about 25 days or about 30 days, about 35 days, about 40 days, about 45 days or about 50 days or more following initiation of immunosuppressive therapy.
50. The method of any of paragraphs 34-49, wherein the transplant is a cardiac allograft transplant or kidney allograft transplant.
51. The method of paragraph 50, wherein the transplant is a cardiac allograft transplant, wherein the method reduces chronic cardiac allograft vasculopathy (CAV).
52. The method of any of paragraphs 34-51, wherein the administration of pro-tolerogenic microbes modulates gut microbiota and metabolic activities.
53. The method of any of paragraphs 34-52, wherein the administration of pro-tolerogenic microbes modulates gut microbiota and metabolic activities to enhance regulatory T cell induction and suppress effector T cell activation.
54. The method of any of paragraphs 34-53, wherein the administration restores gut microbiota balance or diversity.
55. The method of any of paragraphs 34-54, wherein the administration alters laminin α4:α5 ratios in lymph nodes to promote immune tolerance.
56. The method of any of paragraphs 34-55, wherein the administration restores amino acid metabolism, short-chain fatty acid production, and polyamine synthesis in gut microbiota.
57. The method of any of paragraphs 34-56, wherein the administration reduces vascular inflammation and promotes graft survival.
58. The method of any of paragraphs 34-57, wherein the administration reduces graft inflammation and fibrosis.
59. The method of any of paragraphs 34-59, wherein the method further comprises administering a composition comprising an effective amount of pro-tolerogenic microbes to the subject.
60. The method of paragraph 59, wherein the pro-tolerogenic microbes comprise one or more of Akkermansiaceae, Bacteroidaceae, Enterobacteriaceae, Clostridia (cluster XIVa and IV), Lactobacilluseae, Lachnospiraceae, Eubacterium, Bifidobacterium, CAG-180 (Oscillospirales), UBA2882, Bacteroides, Prevotella, Ruminiclostridium (Ruminococcaceae), Bacteroides uniformis, Bacteroides vulgatus, Parabacteroides, Blautia, Butyricimonas, Anaerostipes, Hungatella, COE1, 1XD8-76, Ruminococcus and UBA-7182.
61. The method of paragraph 60, wherein the pro-tolerogenic microbes comprises Bifidobacterium, wherein the Bifidobacterium is Bifidobacterium longum or Bifidobacterium pseudolongum.
62. The method of paragraph 61, wherein the Bifidobacterium is Bifidobacterium longum.
63. The method of any of paragraphs 59-62, wherein the pro-tolerogenic microbes comprise a fecal microbiota transfer (FMT).
64. The method of paragraph 63, wherein the FMT is sourced from a human transplant recipient(s) with a tolerogenic immune regulatory profile, wherein the FMT induces metabolic and/or immune tolerance changes that promote graft survival.
65. The method of any of paragraphs 34-64, wherein the method further comprises stopping the immunosuppressant therapy in the subject and administering to the subject a different immunosuppressant therapy.
66. The method of paragraph 65, wherein the different immunosuppressant therapy comprises administering an effective amount of mycophenolate mofetil (MMF), and/or fingolimod (FTY).
67. A method of reducing transplant rejection in a subject undergoing an immunosuppressant therapy, comprising
i) detecting a dysbiotic state in the subject's gut microbiota resulting from the immunosuppressant therapy; and
ii) stopping the immunosuppressant therapy in the subject and administering to the subject a different immunosuppressant therapy.
68. The method of paragraph 67, wherein the different immunosuppressant therapy comprises administering an effective amount of mycophenolate mofetil (MMF), and/or fingolimod (FTY).
69. The method of paragraph 67 or 68, further comprising administering a therapy to the subject to attenuate the dysbiotic state in the subject's gut microbiota.
70. The method of any of paragraphs 67-69, further comprising detecting the presence or absence of a dysbiotic state in the subject's gut microbiota after administering the different immunosuppressant therapy or the therapy to attenuate the dysbiotic state in the subject's gut microbiota.
71. The method of any of paragraphs 67-70, wherein detecting the dysbiotic state comprises analyzing the gut microbiota for an increase, relative increase, decrease, or relative decrease in one or more microbes.
72. The method of any of paragraphs 67-71, wherein the subject's gut microbiota is analyzed for a relative increase in the presence of one or more pathobionts.
73. The method of paragraph 72, wherein the one or more pathobionts belong to the family of Muribaculaceae.
74. The method of any of paragraphs 67-73, wherein detecting the dysbiotic state comprises analyzing the subject's metabolome profile for an increase, relative increase, decrease, or relative decrease in one or more metabolites (for example, in FIGS. 12C, 13A, 13B, 13C, or 13D).
75. The method of any of paragraphs 67-74, wherein the detecting comprises detecting the presence, absence or relative amounts of one or more differentially expressed genes (for example, in FIG. 2C, Table 1, Table 2, Table 3 or Table 4).
76. The method of any of paragraphs 67-75, wherein the detecting comprises analyzing the lymph nodes in the subject.
77. The method of paragraph 76, wherein the lymph nodes have a pro-inflammatory architecture, characterized by reduced Treg presence and/or decreased laminin α4:α5 ratios.
78. The method of paragraph 76 or 77, wherein mesenteric lymph nodes are analyzed.
79. The method of paragraph 76 or 77, wherein peripheral lymph nodes are analyzed.
80. The method of any of paragraphs 67-79, wherein the immunosuppressive therapy comprises one or more immunosuppressive agent(s) selected from tacrolimus, mycophenolate mofetil, mycophenolic acid, azathioprine, prednisone, fingolimod, rapamycin, cyclosporine, sirolimus, everolimus, and belatacept.
81. The method of any of paragraphs 67-80, wherein the immunosuppressant therapy comprises a combination of tacrolimus, mycophenolate and prednisone.
82. The method of any of paragraphs 67-81, wherein the dysbiotic state is detected about 7 days, about 10 days, about 15 days, about 20 days, about 25 days or about 30 days, about 35 days, about 40 days, about 45 days or about 50 days or more following initiation of immunosuppressive therapy.
83. The method of any of paragraphs 67-82, wherein the transplant is a cardiac allograft transplant or kidney allograft transplant.
84. The method of paragraph 83, wherein the transplant is a cardiac allograft transplant, wherein the method reduces chronic cardiac allograft vasculopathy (CAV).
85. The method of any of paragraphs 69-84, wherein the therapy that attenuates the dysbiotic state in the subject's gut microbiota comprises administration of pro-tolerogenic microbes.
86. The method of any of paragraphs 69-85, wherein the therapy that attenuates the dysbiotic state in the subject's gut microbiota modulates gut microbiota and metabolic activities to enhance regulatory T cell induction and suppress effector T cell activation.
87. The method of any of paragraphs 69-86, wherein the therapy that attenuates the dysbiotic state in the subject's gut microbiota restores gut microbiota balance or diversity.
88. The method of any of paragraphs 69-87, wherein the therapy that attenuates the dysbiotic state in the subject's gut microbiota alters laminin α4:α5 ratios in lymph nodes to promote immune tolerance.
89. The method of any of paragraphs 69-88, wherein the therapy that attenuates the dysbiotic state in the subject's gut microbiota restores amino acid metabolism, short-chain fatty acid production, and polyamine synthesis in gut microbiota.
90. The method of any of paragraphs 69-89, wherein the therapy that attenuates the dysbiotic state in the subject's gut microbiota reduces vascular inflammation and promotes graft survival.
91. The method of any of paragraphs 69-90, wherein the therapy that attenuates the dysbiotic state in the subject's gut microbiota reduces graft inflammation and fibrosis.
92. The method of any of paragraphs 85-91, wherein the pro-tolerogenic microbes comprise one or more of Akkermansiaceae, Bacteroidaceae, Enterobacteriaceae, Clostridia (cluster XIVa and IV), Lactobacilluseae, Lachnospiraceae, Eubacterium, Bifidobacterium, CAG-180 (Oscillospirales), UBA2882, Bacteroides, Prevotella, Ruminiclostridium (Ruminococcaceae), Bacteroides uniformis, Bacteroides vulgatus, Parabacteroides, Blautia, Butyricimonas, Anaerostipes, Hungatella, COE1, 1XD8-76, Ruminococcus and UBA-7182.
93. The method of paragraph 92, wherein the pro-tolerogenic microbes comprises Bifidobacterium, wherein the Bifidobacterium is Bifidobacterium longum or Bifidobacterium pseudolongum.
94. The method of paragraph 93, wherein the Bifidobacterium is Bifidobacterium longum.
95. The method of any of paragraphs 85-94, wherein the pro-tolerogenic microbes comprise a fecal microbiota transfer (FMT).
96. The method of paragraph 95, wherein the FMT is sourced from a human transplant recipient(s) with a tolerogenic immune regulatory profile, wherein the FMT induces metabolic and/or immune tolerance changes that promote graft survival.
97. The method of any of paragraphs 69-96, wherein the therapy that attenuates the dysbiotic state in the subject's gut microbiota comprises an effective amount of a metabolomic composition.
98. The method of paragraph 97, wherein the metabolomic composition comprises one or more of polyamines, short-chain fatty acids, and secondary bile acids.
99. The method of any of paragraphs 97 or 98, wherein the metabolomic composition is derived from gut microbiota.
100. The method of any of paragraphs 97-99, wherein the metabolomic composition comprises an effective amount of spermidine and/or spermine.
101. A method of reducing transplant rejection in a subject undergoing an immunosuppressant therapy, comprising
i) detecting a dysbiotic state in the subject's gut microbiota resulting from the immunosuppressant therapy; and
ii) administering to the subject a therapy that attenuates the dysbiotic state in the subject's gut microbiota.
102. The method of paragraph 101, further comprising detecting the presence or absence of a dysbiotic state in the subject's gut microbiota after administering the therapy that attenuates the dysbiotic state in the subject's gut microbiota.
103. The method of paragraph 101 or 102, wherein detecting the dysbiotic state comprises analyzing the gut microbiota for an increase, relative increase, decrease, or relative decrease in one or more microbes.
104. The method of any of paragraphs 101-103, wherein the subject's gut microbiota is analyzed for a relative increase in the presence of one or more pathobionts.
105. The method of paragraph 104, wherein the one or more pathobionts belong to the family of Muribaculaceae.
106. The method of any of paragraphs 101-105, wherein detecting the dysbiotic state comprises analyzing the subject's metabolome profile for an increase, relative increase, decrease, or relative decrease in one or more metabolites (for example, in FIGS. 12C, 13A, 13B, 13C, or 13D).
107. The method of any of paragraphs 101-106, wherein the detecting comprises detecting the presence, absence or relative amounts of one or more differentially expressed genes (for example, in FIG. 2C, Table 1, Table 2, Table 3 or Table 4).
108. The method of any of paragraphs 101-107, wherein the detecting comprises analyzing the lymph nodes in the subject.
109. The method of paragraph 108, wherein the lymph nodes have a pro-inflammatory architecture, characterized by reduced Treg presence and/or decreased laminin α4:α5 ratios.
110. The method of paragraph 108 or 109, wherein mesenteric lymph nodes are analyzed.
111. The method of paragraph 108 or 109, wherein peripheral lymph nodes are analyzed.
112. The method of any of paragraphs 101-111, wherein the immunosuppressive therapy comprises one or more immunosuppressive agent(s) selected from tacrolimus, mycophenolate mofetil, mycophenolic acid, azathioprine, prednisone, fingolimod, rapamycin, cyclosporine, sirolimus, everolimus, and belatacept.
113. The method of any of paragraphs 101-112, wherein the immunosuppressant therapy comprises a combination of tacrolimus, mycophenolate and prednisone.
114. The method of any of paragraphs 101-113, wherein the dysbiotic state is detected about 7 days, about 10 days, about 15 days, about 20 days, about 25 days or about 30 days, about 35 days, about 40 days, about 45 days or about 50 days or more following initiation of immunosuppressive therapy.
115. The method of any of paragraphs 101-114, wherein the transplant is a cardiac allograft transplant or kidney allograft transplant.
116. The method of paragraph 115, wherein the transplant is a cardiac allograft transplant, wherein the method reduces chronic cardiac allograft vasculopathy (CAV).
117. The method of any of paragraphs 101-116, wherein the therapy that attenuates the dysbiotic state in the subject's gut microbiota comprises administration of pro-tolerogenic microbes.
118. The method of any of paragraphs 101-117, wherein the therapy that attenuates the dysbiotic state in the subject's gut microbiota modulates gut microbiota and metabolic activities to enhance regulatory T cell induction and suppress effector T cell activation.
119. The method of any of paragraphs 101-118, wherein the therapy that attenuates the dysbiotic state in the subject's gut microbiota restores gut microbiota balance or diversity.
120. The method of any of paragraphs 101-119, wherein the therapy that attenuates the dysbiotic state in the subject's gut microbiota alters laminin α4:α5 ratios in lymph nodes to promote immune tolerance.
121. The method of any of paragraphs 101-120, wherein the therapy that attenuates the dysbiotic state in the subject's gut microbiota restores amino acid metabolism, short-chain fatty acid production, and polyamine synthesis in gut microbiota.
122. The method of any of paragraphs 101-121, wherein the therapy that attenuates the dysbiotic state in the subject's gut microbiota reduces vascular inflammation and promotes graft survival.
123. The method of any of paragraphs 101-122, wherein the therapy that attenuates the dysbiotic state in the subject's gut microbiota reduces graft inflammation and fibrosis.
124. The method of any of paragraphs 117-123, wherein the pro-tolerogenic microbes comprise one or more of Akkermansiaceae, Bacteroidaceae, Enterobacteriaceae, Clostridia (cluster XIVa and IV), Lactobacillusceae, Lachnospiraceae, Eubacterium, Bifidobacterium, CAG-180 (Oscillospirales), UBA2882, Bacteroides, Prevotella, Ruminiclostridium (Ruminococcaceae), Bacteroides uniformis, Bacteroides vulgatus, Parabacteroides, Blautia, Butyricimonas, Anaerostipes, Hungatella, COE1, 1XD8-76, Ruminococcus and UBA-7182.
125. The method of paragraph 124, wherein the pro-tolerogenic microbes comprises Bifidobacterium, wherein the Bifidobacterium is Bifidobacterium longum or Bifidobacterium pseudolongum.
126. The method of paragraph 125, wherein the Bifidobacterium is Bifidobacterium longum.
127. The method of any of paragraphs 117-126, wherein the pro-tolerogenic microbes comprise a fecal microbiota transfer (FMT).
128. The method of paragraph 127, wherein the FMT is sourced from a human transplant recipient(s) with a tolerogenic immune regulatory profile, wherein the FMT induces metabolic and/or immune tolerance changes that promote graft survival.
129. The method of any of paragraphs 102-128, wherein the therapy that attenuates the dysbiotic state in the subject's gut microbiota comprises an effective amount of a metabolomic composition.
130. The method of paragraph 129, wherein the metabolomic composition comprises one or more of polyamines, short-chain fatty acids, and secondary bile acids.
131. The method of any of paragraphs 129 or 130, wherein the metabolomic composition is derived from gut microbiota.
132. The method of any of paragraphs 129-131, wherein the metabolomic composition comprises an effective amount of spermidine and/or spermine.
133. A method of screening for microbial strains associated with improved transplant outcomes, comprising isolating and characterizing gut microbiota from transplant recipients with a tolerogenic immune regulatory profile, and identifying strains that promote regulatory T cell induction and suppress effector T cell activation.
134. A method of reducing transplant rejection in a subject undergoing an immunosuppressant therapy, comprising administering to the subject an effective amount of pro-tolerogenic microbes that attenuate a dysbiotic state in the subject's gut microbiota.
135. A method of reducing transplant rejection in a subject undergoing an immunosuppressant therapy, comprising administering to the subject an effective amount of a metabolomic composition that attenuates a dysbiotic state in the subject's gut microbiota.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Immunosuppressants Rewire the Gut-Alloimmune Axis Through Time-Dependent and Tissue-Specific Mechanisms

In this example, the present inventors investigated the understudied effects of immunosuppressants on LN architecture, lymphocyte and myeloid cell distribution, and the gut microbiome. Four key classes of clinical immunosuppressants were examined: the calcineurin inhibitor tacrolimus (TAC), the glucocorticoid prednisone (PRED), the inosine monophosphate dehydrogenase (IMPDH) inhibitor MMF, and the sphingosine-1-phosphate (SIP) receptor antagonist fingolimod (FTY). Results presented herein reveal complex, time-dependent and tissue-specific patterns in drug responses and strikingly convergent gut microbial and intestinal responses. Despite their distinct mechanisms of action and not designed to target the gut, all drugs induced progressive alterations from moderate early changes to substantial alterations with prolonged treatment, converging to a shared dysbiotic state in the gut microbiome marked by significant expansion of pathobionts of Muribaculaceae across all drug classes. This was accompanied by significant metabolic alterations and distinct phases of intestinal transcriptional responses. Time-dependent changes were also observed in LNs. The mesenteric LNs (mLNs) developed progressively increased pro-inflammatory states with extended immunosuppressive use, while peripheral LNs (pLNs) showed strong early pro-tolerogenic effects that became less pronounced over time. While this compartmentalized immune regulation was broadly consistent across all drugs studied, MMF and FTY demonstrated particularly robust immunomodulatory effects. These two agents effectively suppressed alloantigen-induced pro-inflammatory changes, through mediating Treg redistribution and LN remodeling. Overall these findings revealed the underappreciated complexity and dynamics of immunosuppressant effects, suggesting a mechanistic link between immunosuppressant-induced gut dysbiosis, compartmentalized immune regulation in peripheral and mesenteric lymphoid tissues, and immunosuppressant off-target complications.

### Results

### Time-Dependent Effects of Immunosuppressants on Gut Microbiome

We treated groups of C57BL/6 mice daily with four immunosuppressants (TAC, PRED, MMF and FTY), respectively, using untreated mice as controls. We analyzed the gut microbiome at early (3 days), intermediate (7 days), and late (30 days) time points. We performed whole community metagenomic sequencing on colonic intraluminal stool samples and utilized the comprehensive mouse gut metagenome catalogue (CMGM) for taxonomic profiling and Human Microbiome Project Unified Metabolic Analysis Network (HUMAnN3) to analyze microbial metabolic pathways (Kieser et al., bioRxiv, (2021), 2021:2003.2018.435958; Franzosa et al., Nat Methods, (2018), 15:962-968).

To quantify immunosuppressant drug effects on the gut microbiome, we used Bray-Curtis distance analyses to measure changes in both taxonomic composition and metabolic pathways (FIG. 1A, B). We compared each treatment group to untreated controls (treatment-to-control) to determine deviations from baseline and analyzed differences between different treatment groups (treatment-to-treatment). In treatment-to-control pairwise comparisons, the effects were variable during early treatment by day 3 and 7.

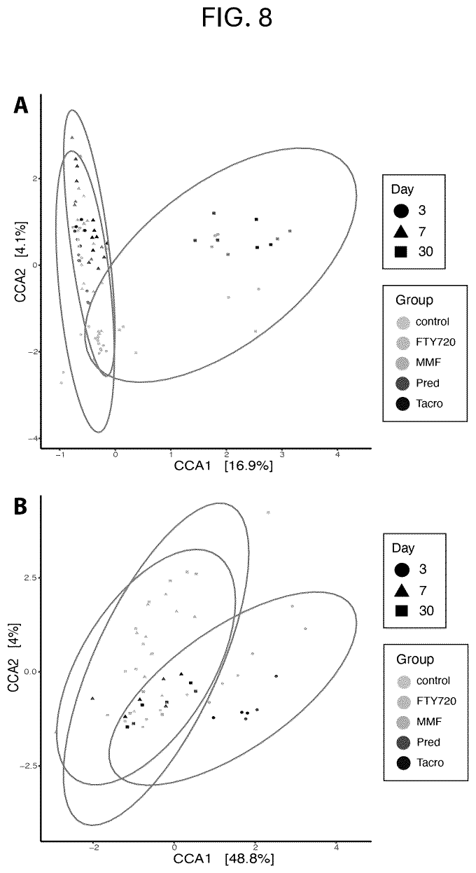

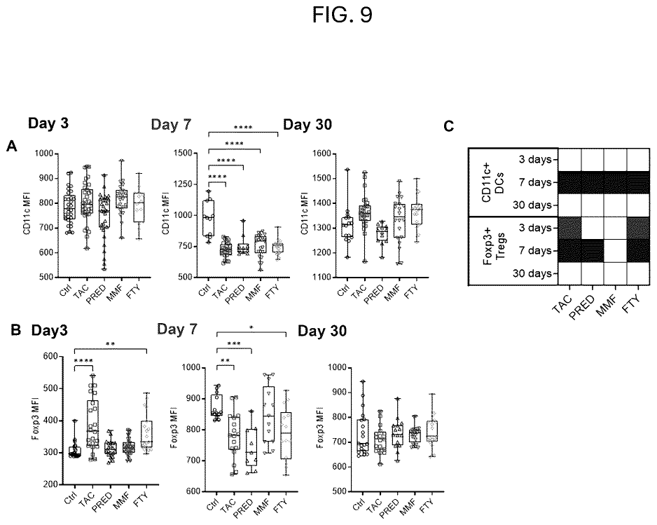

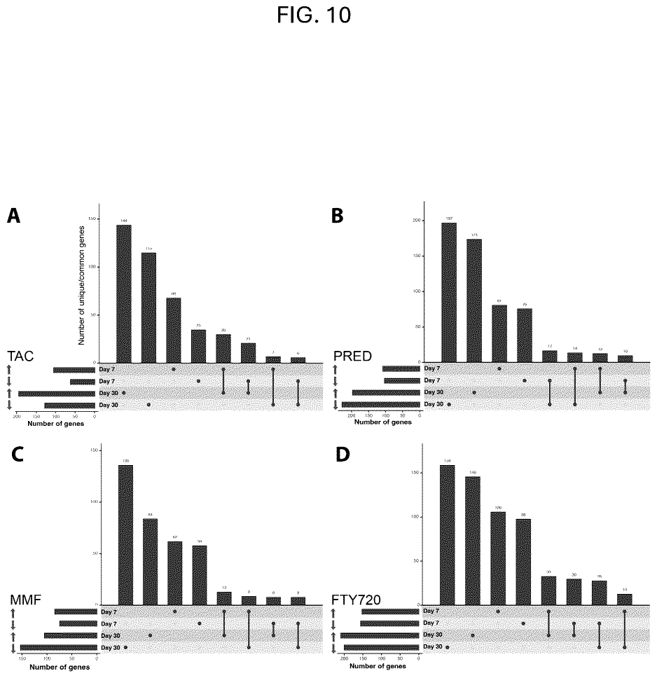

However, by day 30, all treatment groups showed profound and significant alterations in their gut microbiome profiles, suggesting a cumulative drug effect with prolonged treatment. The pattern was evident in both taxonomic compositions and functional pathways, with the most substantial changes emerging with 30-day treatment. Drug-to-drug pairwise comparison also revealed distinct time-dependent drug effects on gut microbiome. The differences between treatment groups increased from day 3 to day 7, indicating drug-specific early effects on microbial communities. However, by day 30, these inter-drug differences had substantially decreased, suggesting that despite their distinct mechanisms of action, prolonged immunosuppression lead to convergent effects on the gut microbiome. These findings were further supported by ordination analyses. Principal Component Analysis (PCA) revealed clear clustering of 30-day samples, distinct from earlier time points, regardless of the specific immunosuppressant used (FIG. 1C). Canonical Correspondence Analysis (CCA) confirmed these observations, demonstrating significant correlations between treatment duration and microbiome composition, with maximal separation occurring at 30 days (FIGS. 8A and 8B).

The temporal convergence of microbiome alterations across different immunosuppressant drugs revealed shared downstream influences of these drugs on host-microbe interactions, despite their diverse initial effects. To identify microbial signatures associated with immunosuppression, we compared the abundance of bacterial species between drug-treated and control groups across multiple timepoints to reveal treatment-specific and temporal changes in microbial populations (FIG. 1D). No individual taxonomic group showed consistent alterations across all timepoints in any treatment group. Early (day 3) and intermediate (day 7) time points revealed sporadic alterations in various bacterial taxa, but these changes were transient. By day 30, all treatment groups, despite their distinct mechanisms of action, converged on a common set of bacterial taxa that were not significant at the earlier time points. These taxa belonged exclusively to the family of Muribaculaceae (formerly known as S24-7), including Duncaniella, Paramuribaculum intestinale, and CAG-873. This family is known for its metabolic versatility and adaptability, and is primarily a gut commensal in mice but can act as pathobionts under dysbiotic conditions, with recent evidence demonstrating their causative role in insulin-dependent diabetes in mouse models (Ormerod et al., Microbiome, (2016), 4:36; Lagkouvardos et al., Microbiome, (2019), 7:28; Smith et al., mSphere, (2021), 6:e0085121; Yang et al., Microbiome, (2023), 11:62). Their enrichment indicates a shift toward a dysbiotic state in which the microbial balance is skewed toward species with pathobiont potentials with altered metabolic capabilities. This progression from early drug-specific changes to a convergent dysbiotic state with prolonged treatment underlines the profound and progressive impact of chronic immunosuppressant use on the gut microbiome. Conserved intestinal responses to immunosuppressants revealed induction of epithelial stress and suppression of lymphoid signatures

To understand host gut responses to drug treatment, we analyzed the transcriptome of small intestine at days 7 and 30, as these time points corresponded to significant alterations in the gut microbiome during treatments. Differentially expressed genes (DEGs) were identified by comparing each drug treatment group to the no-treatment controls. DEGs analysis revealed that 30-day treatments induced 1.3 to 2 times more DEGs than 7-day treatments (FIG. 2A), indicating that prolonged immunosuppression triggered more extensive transcriptional changes. At day 7, FTY induced the largest number of DEGs. By day 30, both FTY and PRED showed stronger effects in both upregulation and downregulation of DEGs. In contrast, MMF showed the least impact, particularly in upregulated DEGs. TAC induced the largest portion of upregulated DEGs with twice as many upregulated DEGs as downregulated ones, compared to other drugs that showed either greater downregulation or balanced regulation. Overall, prolonged immunosuppressive treatment drove broader and more extensive transcriptional changes in intestinal tissue.

We next analyzed DEGs common to all immunosuppressant treatments to identify shared drug-induced effects (Tables 1-4). Table 1 shows genes upregulated after 7 days of treatment with FTY, MMF, Pred, Rapa or Tac. Table 2 shows genes upregulated after 30 days of treatment with FTY, MMF, Pred, Rapa or Tac. Table 3 shows genes downregulated after 7 days of treatment with FTY, MMF, Pred, Rapa or Tac. Table 4 shows genes downregulated after 30 days of treatment with FTY, MMF, Pred, Rapa or Tac. To enable a comprehensive comparison across different immunosuppressant classes, we integrated transcriptomic data from rapamycin-treated mice from our previously study into the current analysis (FIG. 2B) (Wu et al., JCI Insight, (2025), 10:10.1172/jci.insight.186505). Conserved DEGs were defined as genes consistently differentially expressed by at least four drugs at either time points. Clustering analysis of these conserved DEGs at days 7 and 30 revealed distinct temporal expression patterns, particularly affecting genes involved in mucosal immunity, epithelial homeostasis, metabolism, and circadian regulation (FIG. 2C).

Specifically, immunoglobulin heavy variable (Ighv) and kappa light chain variable (Igkv) region genes displayed broad yet temporally distinct responses under immunosuppressant treatments. Cluster 1 DEGs (FIG. 2B left panel) includes genes mainly suppressed at day 30, including classical Ighv and Igkv segments typically associated with naive and T-dependent germinal center-derived B cells. This downregulation indicates a significant, prolonged suppression of de novo B cell activation and diversification, especially affecting clones that require antigen-specific, T cell-dependent signals. Cluster 2 DEGs were mostly suppressed at day 7 only. These genes, which include variable region segments commonly expressed by naive or early responding B cells, suggest an early transcriptional quiescence. In contrast, Cluster 3 genes were initially suppressed at day 7 but later upregulated at day 30. The activation of the Ighg2b gene suggests that class-switch recombination has occurred, reflecting the selective survival and expansion of mucosal or microbiota-tolerant B cell subsets capable of adapting to the chronically immunosuppressed gut. Overall, these results indicate a predominantly suppressed B cell transcriptional program, with limited recovery by day 30.

Further immunohistochemical (IHC) assessments of intestinal conventional dendritic cells (CD11c+ cDCs) and regulatory T cells (Foxp3+ Tregs) revealed day 7 was the critical transitional point for all immunosuppressant drugs (FIG. 9A-C). A significant reduction in intestinal cDC populations at day 7 was observed for all drugs, with no significant alterations observed at days 3 or 30. Intestinal Tregs similarly had the most significant decreases at day 7, while returning to baseline levels by day 30. Collectively, these results indicated that at day 7, B cells experienced diminished antigenic stimulation, reduced survival and differentiation signals, and suppressed transcription of immunoglobulin variable region genes. By day 30, limited restoration of antigen delivery, B and T cell costimulatory signals, and epithelial tolerance to baseline levels associated with partial resumption of recombined immunoglobulin gene transcription, especially among surviving tissue-resident B cells. This partially restored yet still broadly suppressed state may represent a compensatory host response, favoring class-switched memory or microbiota-reactive B cell clones to maintain mucosal homeostasis under ongoing immunosuppressive therapy.

Epithelial DEGs showed three distinct temporal expression clusters, reflecting the mucosal defense and host compensatory responses to immunosuppressant treatment. Cluster 1 DEGs were significantly activated by day 30, indicative of robust mucosal defense mechanisms and adaptive metabolic programs. Outstandingly, this cluster included key antimicrobial effectors, including α-defensins (Defa-ps16, Defa-ps7, Defa2, Defa20-22, Defa29, Defa3, Defa32, Defa34-36, Defa40-42, Defa5), lysozyme (Lyz1), and secretory phospholipase A2 group IIA (Pla2g2a), which are canonical products of Paneth cells (Bevins et al., Nat Rev Microbiol, (2011), 9:356-368). This transcriptional profile is consistent with a Paneth cell-driven antimicrobial response, suggesting that activation of innate epithelial defense mechanisms was possibly triggered by pathobiont expansion and microbiota perturbation following prolonged immunosuppressant exposure. Further, oxidase genes (Duox2, Duoxa2) were elevated, supporting reactive oxygen species (ROS) production for microbial control. Moreover, upregulation of inducible nitric oxide synthase (iNOS, Nos2) and glutathione peroxidase (Gpx2) suggested enhanced epithelial stress responses. These adaptive responses likely represented host mechanisms to control potential microbiota perturbations. Cluster 2 genes, mainly upregulated at day 7, prominently included key circadian regulators (Ciart, Per1, Per2, Per3, Hlf), suggesting an early host-driven resetting of circadian rhythms in response to drug-induced gut perturbation. Further, genes involved in xenobiotic metabolism and detoxification pathways (Cyp26b1, Cesld, Sult6b2) and early immune interaction genes (Cd19, Fcer2a, Pax5) were also significantly induced. Together these genes represented an early epithelial, metabolic and immune adaptation to drug disturbance. Cluster 3 genes were downregulated at day 30, including key genes involved in epithelial differentiation and renewal (Onecut2, Tmem229b), lipid biosynthesis (Srebf1), extracellular matrix remodeling (Chst3, Adamts4), and mast cell proteases (Mcpt1, Cma2). The suppression of these functions reflected a compromised capacity for epithelial regeneration and reduced structural adaptability after prolonged treatment. Overall, these data showed a profound off-target influence of the immunosuppressant drugs on the intestine, and importantly, a dynamic mucosal adaptation to immunosuppressive induction, transitioning from initial circadian and xenobiotic metabolism, progressing toward robust innate mucosal defense programs and suppression of epithelial renewal and structural remodeling processes.

### Drug-Specific Alterations in Intestinal Expression and Gut Luminal Metabolic Activity

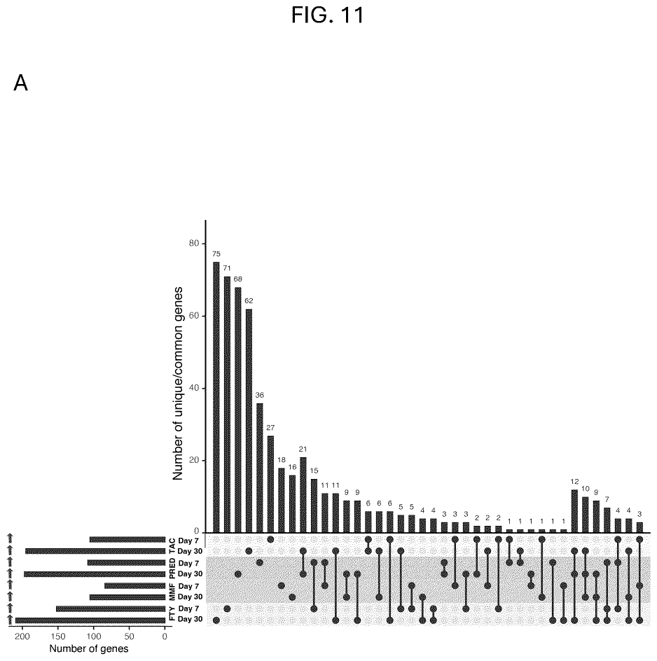

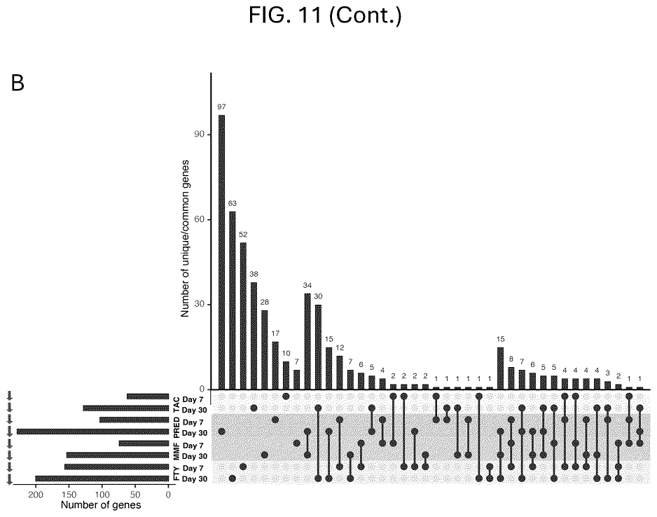

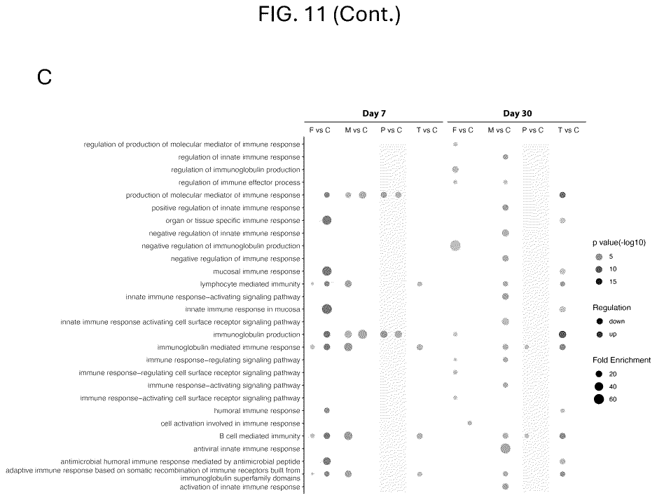

Beyond shared mechanisms, we next examined drug-specific transcriptional programs to identify unique intestinal responses elicited by individual immunosuppressant agents. UpSet plots were used to analyze DEGs unique to or shared between days 7 and 30, as well as across different treatment groups (FIG. 10A-D). Drug effects were more pronounced at day 30, with 1.6-3.3 times more down-regulated DEGs and 1.4-2.2 times more upregulated DEGs compared to day 7. Among the treatments, TAC showed the most pronounced effect at day 30, while FTY had the least impact. Further, minimal overlap in DEGs was observed between timepoints and across different treatments, indicating transient effects at day 7. For instance, following PRED treatment, only 31 genes (15 upregulated, 16 downregulated) were consistently modulated at both timepoints, while 255 genes were uniquely regulated at day 7 (81 upregulated, 78 downregulated) and 371 at day 30 (174 upregulated, 197 downregulated). This pattern held true for all four drug treatments. Further comparison between treatment groups revealed distinct patterns of gene expression (FIG. 11A-B). The overlap in DEGs was limited, with less than 20% between any two drugs and less than 5% across the four drugs, underscoring highly drug-specific mechanisms of action on intestine. Together, these findings demonstrated that immunosuppressants induced time-dependent intestinal responses, with most pronounced drug-specific changes following prolonged treatment.

Gene ontology enrichment analyses of DEGs revealed drug-specific patterns (FIG. 11C). At day 7, FTY showed the strongest suppressive effect on mucosal and humoral immunity pathways, which transitioned to moderate activation of immunoglobulin production by day 30. MMF shifted from early activation on day 7 to broad suppression of innate immune pathways at day 30. TAC showed moderate increases in lymphocyte and immunoglobulin pathways at day 7, progressing to stronger activation of both innate immunity and antibody production at day 30. PRED maintained relatively modest effects throughout the treatment period, though the specific pathways affected differed between the two timepoints. Together, these data demonstrated that immunosuppressant drugs exerted both shared and drug-specific effects on the intestine, profoundly impacting epithelial function as well as innate and adaptive immune pathways, contributing to the off-target effects of immunosuppressive therapies.

Gut luminal metabolic activity of small intestine was further analyzed at day 7, as this time point represented a critical transitional phase. Untargeted metabolomic analysis identified 264 distinct metabolites in intraluminal stool samples. Partial Least Squares Discriminant Analysis (PLS-DA) demonstrated separations of metabolic profiles among the different treatment groups (FIG. 12A), and the top contributing metabolites in each group are shown in FIG. 12B. Hierarchical clustering analysis further confirmed these drug-specific patterns, demonstrating that each immunosuppressant induced unique changes in metabolite abundance (FIG. 12C). Pairwise comparison of individual drugs to no-treatment controls, combined with enrichment analyses, revealed both shared and drug-specific effects on metabolic activities (Table 5).

TABLE 5
Differential metabolite abundance in gut lumen
after 7 days of immunosuppressant treatment *

| | TAC | PRED | MMF | FTY |
|---|---|---|---|---|
| ↑ | 31 | 4 | 4 | 14 |
| | Amino acids & | | | Purine metabolism |
| | derivatives | | | Arginine metabolism |
| | Pyrimidine | | | |
| | metabolism | | | |
| | Sugars and sugar | | | |
| | phosphates | | | |
| ↓ | 28 | 95 | 37 | 22 |
| | Polyamines & | Amino acids & | Glyoxylate and | Pyrimidine |
| | derivatives | derivatives | Dicarboxylate | metabolism |
| | SCFAs, TMA | SCFAS, TMA | Branched-chain | SCFAs |
| | | Pyrimidine | amino acids & | |
| | | metabolism | derivatives | |
| | | Polyamines & | Purine metabolism | |
| | | derivatives | Neurotransmitters | |
| subtotal | 59 | 99 | 41 | 36 |

* Gut luminal metabolites with significant changes (fold change >2, p < 0.05) after 7 days of treatment with tacrolimus (TAC), prednisone (PRED), mycophenolate mofetil (MMF), or Fingolimod (FTY) compared to untreated controls. Numbers in bold indicate total metabolites significantly increased (↑) or decreased (↓) for each drug. Key metabolic pathways affected are listed for each drug. SCFAs: short-chain fatty acids; TMA: trimethylamine. Data derived from untargeted metabolomics analysis using capillary electrophoresis-mass spectrometry.

TAC treatment induced similar numbers of significantly increased and decreased metabolites (FIG. 13A). Metabolites elevated by TAC included amino acids and derivatives, suggesting altered protein catabolismand amino acid utilization; pyrimidine metabolites indicative of increased nucleotide turnover, and sugars/sugar phosphates reflecting enhanced glycolytic activity and central carbon metabolism. Conversely, TAC significantly reduced polyamines, biogenic amines, short-chain fatty acids (SCFAs), betaine, trimethylamine (TMA), and various organic acids. In contrast, PRED, MMF, and FTY primarily induced suppressive metabolic effects, each with distinct scope and profiles. PRED caused the broadest suppression, reducing three to four times more metabolites than it increased (FIG. 13B), including amino acid derivatives, SCFAs, TMA, pyrimidine intermediates, and polyamines, consistent with its widespread inhibitory effects on cellular metabolism (van Raalte et al., Eur J Clin Invest, (2009), 39:81-93; Compston, J., Endocrine, (2018), 61:7-16; Eastell et al., J Intern Med, (1998), 244:271-292). MMF showed a similarly suppressive but more targeted profile, predominantly affecting glyoxylate/dicarboxylate metabolism, branched-chain and aromatic amino acids, and purine pathways (FIG. 13C). MMF increased adenosine while reducing uric acid, consistent with its mechanism as an inosine monophosphate dehydrogenase (IMPDH) inhibitor, directly linking metabolic changes to its therapeutic effects (Sintchak et al., Cell, (1996), 85a; 921-930; Heischmann et al., Sci Rep, (2017), 7:45088). FTY induced a relatively milder suppressive profile (FIG. 13D), with increased nucleosides and reduced nucleotides and SCFAs. Notably, decreased arginine and elevated N¹, N⁸-diacetylspermidine and spermidine pointed to enhanced polyamine biosynthesis. Collectively, these findings underscore shared and distinct metabolic alterations in the gut lumen that may contribute to both therapeutic efficacy and off-target effects.

Tissue-specific and time-dependent effects of immunosuppressants on mLN and pLN organization

We next investigated how immunosuppressants affected LN organization in both mucosal and systemic compartments by examining mLN and pLN, the primary draining sites for mucosal and systemic immunity, respectively. IHC provides spatial resolution, enabling localized assessments of Tregs within special areas such as LN HEVs and CRs, whereas flow cytometry offers a quantitative measurement of overall Treg populations. These methods complemented each other, measuring different aspects of immune cell dynamics and distribution. Using flow cytometry and IHC, we analyzed changes in the numbers and distribution of immune cell populations (B220+ B cells, CD4+ T cells, CD8+ T cells, Foxp3+ Tregs) and structural features (La4:La5 in LNs). We particularly assessed the changes to La4:La5 and distribution of Tregs in the CR and around the HEVs of LNs to understand how immunosuppressants modulated cellular and structural aspects of immune organization crucial for immune regulation.

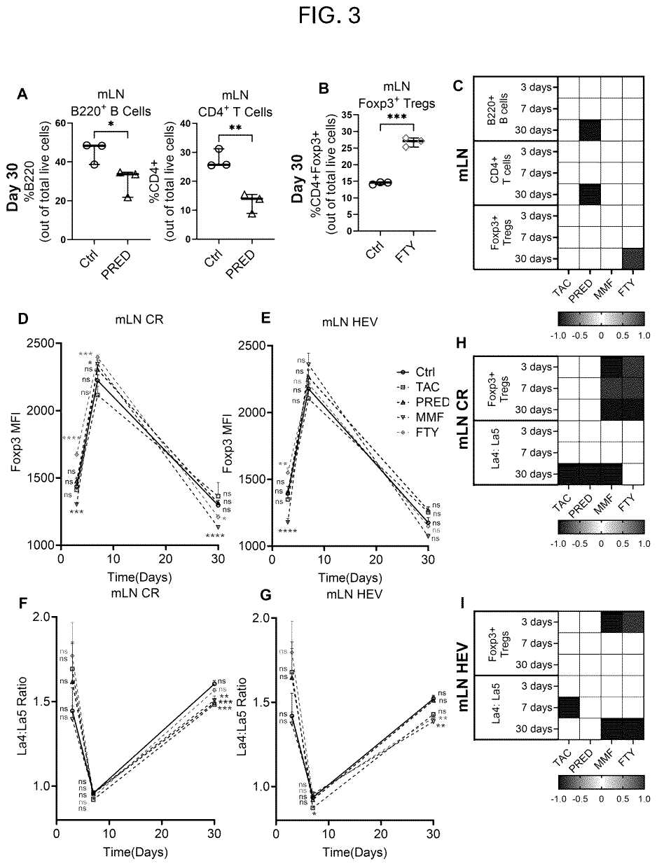

In mLNs, flow cytometry revealed that B220+ B cells, CD4+ T cells, CD8+ T cells and Foxp3+ Tregs were unaffected by TAC or MMF treatment at days 3, 7, or 30; in contrast, PRED treatment significantly reduced CD4+ T cells and B220+B cells by day 30, with no significant changes at earlier time points (FIG. 3A, C). FTY treatment increased Foxp3+ Tregs by day 30, with no changes at days 3 or 7 (FIG. 3B, C). Overall, while CD8+ T cell populations did not significantly change across conditions (data not shown), other populations showed treatment-specific changes that were only evident after prolonged treatment. IHC revealed two groups of change patterns. On the one hand, TAC and PRED showed no significant changes in Treg distribution at any time point (FIG. 3D, E, H, I, FIG. 13A, B), but reduced La4:La5 around the CR after 30 days (FIG. 3F, H, FIG. 13C). TAC additionally decreased this ratio in the HEV on day 7 (FIG. 3G, I, FIG. 13D). On the other hand, MMF and FTY showed similar patterns with an increased Treg distribution in the CR by day 7, followed by a decrease by day 30 (FIG. 3D, H, FIG. 13A) and a decreased La4:La5 in both CR and HEV (FIG. 3F-I, FIG. 13C, D). Early responses at day 3 were mixed, suggesting initial variable effects converged to a stable state with prolonged drug treatment. Overall, despite their distinct mechanisms of action, all four immunosuppressant drugs ultimately induced a more pro-inflammatory environment in mLN by day 30, characterized by reduced Treg presence and/or decreased La4:La5. This temporal progression suggested that prolonged immunosuppression may paradoxically create localized pro-inflammatory conditions in mucosal-associated lymphoid tissues.

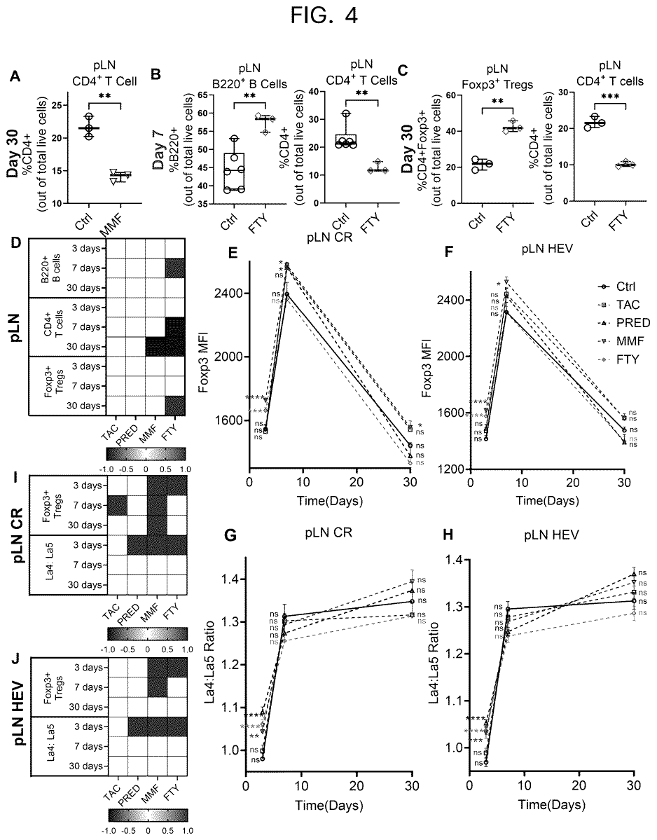

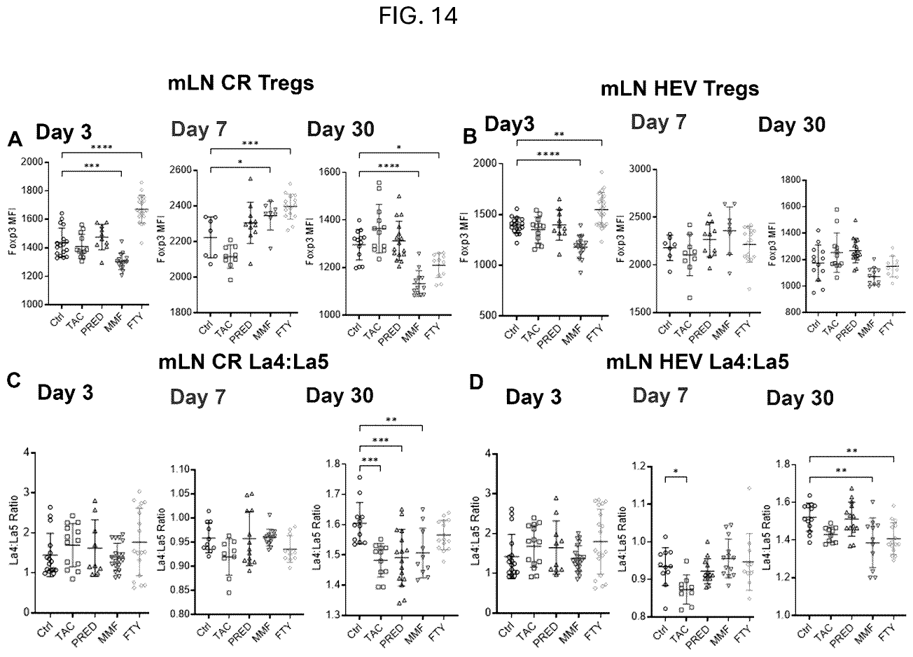

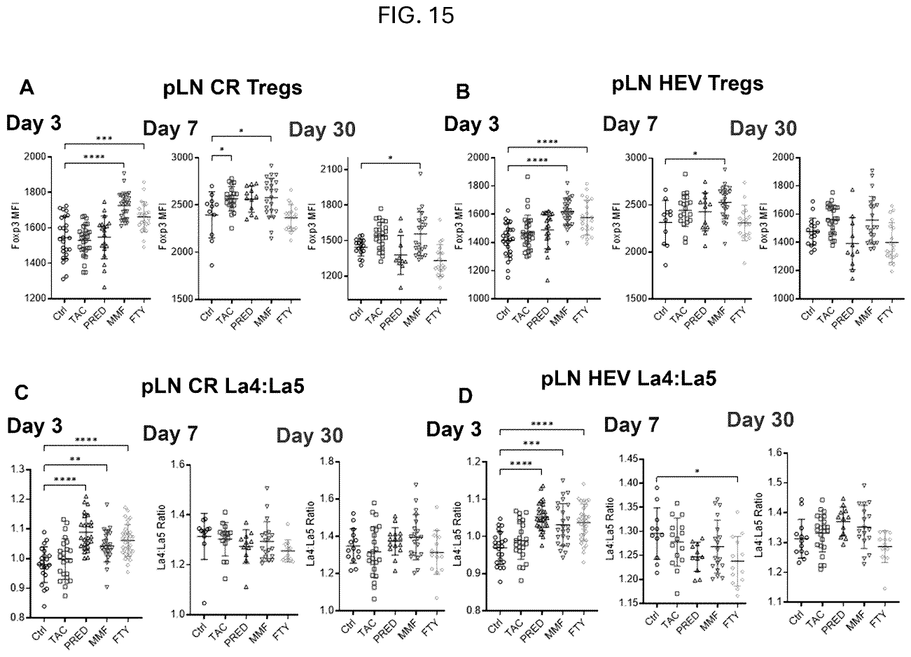

In pLNs, distinct temporal patterns of immune modulation were also observed. Using flow cytometry, TAC and PRED induced minimal effects on cellular composition (FIG. 4D), and MMF selectively reduced CD4+ T cells by day 30 (FIG. 4A, D). FTY showed the most dynamic changes, increasing B220+ B cells while decreasing CD4+ T cells at day 7 (FIG. 4B, D), followed by elevated Foxp3+ Tregs and decreased CD4+ T cells by day 30 (FIG. 4C, D). IHC analysis revealed complementary changes in cellular distribution and stromal architecture. TAC showed transient effects, characterized by increased Treg distribution in the CR at day 7 (FIG. 4E, I, FIG. 14A) without affecting laminin ratios (FIG. 4G-J, FIG. 14C, D). PRED demonstrated only early structural changes, marked by increased La4:La5 at day 3 (FIG. 4E-J, FIG. 14A-D). MMF showed the most consistent pro-tolerogenic profile, maintaining increased Treg distribution throughout the treatment period (FIG. 4E, F, I, J, FIG. 14A, B) and a higher La4:La5 at day 3 (FIG. 4G-J, FIG. 14C, D). FTY showed early pro-tolerogenic effects with increased Treg distribution and La4:La5 at day 3, but not at later time points (FIG. 4E-J, FIG. 14A-D). Together, these findings demonstrated that all four immunosuppressants, particularly MMF and FTY, predominantly promoted a pro-tolerogenic state in pLNs, characterized by increased Tregs and/or elevated laminin α4:α5 ratios particularly during early treatment phases. This contrasted with the pro-inflammatory environment observed in mLNs, suggesting differential regulation of immune organization between loco-regional and systemic lymphoid tissue compartments. MMF and FTY counteract alloantigen-induced pro-inflammatory changes through dual regulation of Tregs and LN architecture

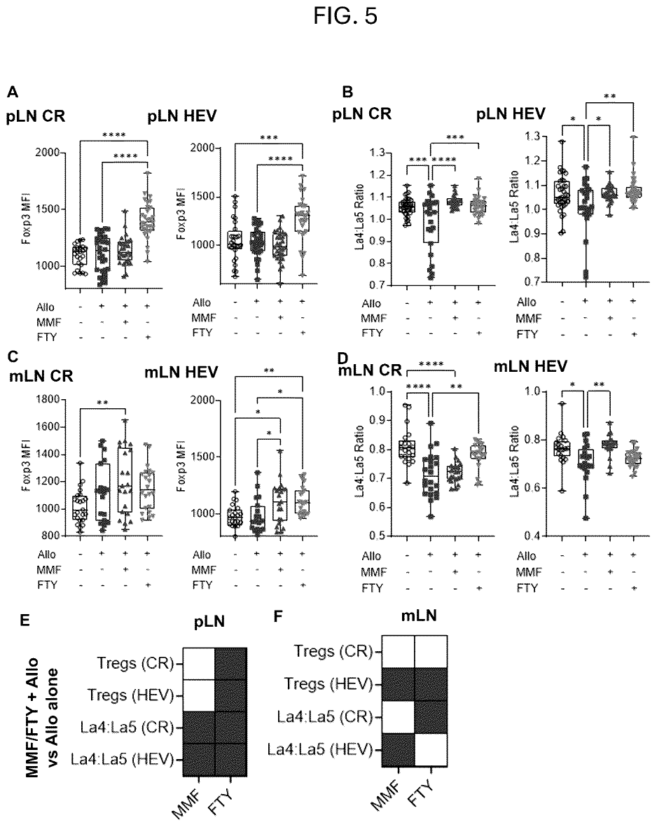

Given the pronounced effects of MMF and FTY on LN organization under homeostatic conditions (FIG. 3H, I, 4I, J), we next utilized a mouse model with allogeneic stimulation (Allo) to assess their impact on transplant-related alloimmune responses, mimicking the immunologic stress of solid organ transplantation. Mice were injected with fully allogeneic splenocytes (10⁷ cells intravenously) followed by MMF or FTY administration for 3 days. Compared to the no treatment controls, Allo alone did not affect Treg distribution in mLNs or pLNs (FIG. 5A, C). However, when combined with either drug, significant increases in Tregs were observed. Compared to Allo alone, MMF increased Tregs around mLN HEV, and FTY increased Tregs in both mLN HEV and PLN HEV and CR (FIG. 5A, C, E, F). Allo alone decreased La4:La5 in both pLN and mLN (FIG. 5B, D), indicating a pro-inflammatory shift caused by alloantigen-induced immune responses, consistent with our previous findings (Simon et al., Transplantation, (2019), 103:2075-2089). When combined with MMF or FTY, both drugs prevented this decrease, maintaining laminin α4:α5 ratios comparable to untreated controls (FIG. 5B, D, E, F). These findings demonstrated that MMF and FTY inhibited alloantigen-induced pro-inflammatory shifts in LN through two complementary mechanisms: increasing Tregs and preserving tolerogenic laminin stromal architecture. This dual action suggested that these drugs may be particularly effective at promoting a tolerogenic environment during allogeneic immune challenges.

MMF and FTY effects are mediated through FRC-derived laminins a4 and a5

Given the significant impact of MMF and FTY on LN architecture, particularly through modulation of La4:La5 under both homeostatic and allogeneic stimulated conditions, we next investigated whether these drugs directly influenced FRC-derived laminins, given the crucial role of FRC in modulating laminin expression (Li, L., Shirkey et al., J Clin Invest, (2020), 130:2602-2619; Warren et al., J Clin Invest, (2014), 124:2204-2218). We utilized two laminin KO mouse strains: FRC-Laminin4-KO mice (Pdgfrb-Cre+/−×La4^{fl/fl}) have the laminin α4 gene specifically deleted in FRCs, and FRC-Laminin5-KO mice (Pdgfrb-Cre+/−×La5^{fl/fl}) have the laminin α5 gene specifically deleted in FRCs (Li, L., Shirkey et al., J Clin Invest, (2020), 130:2602-2619; Li et al., J Clin Invest, (2022), 132:10.1172/JCI156994. FRC-Lama4-KO and FRC-Lama5-KO mice were treated with MMF or FTY for 3 days, to specifically examine whether FRC-derived laminin α4 and a5 played a role in immunosuppressant-mediated effects on LN stromal organization.

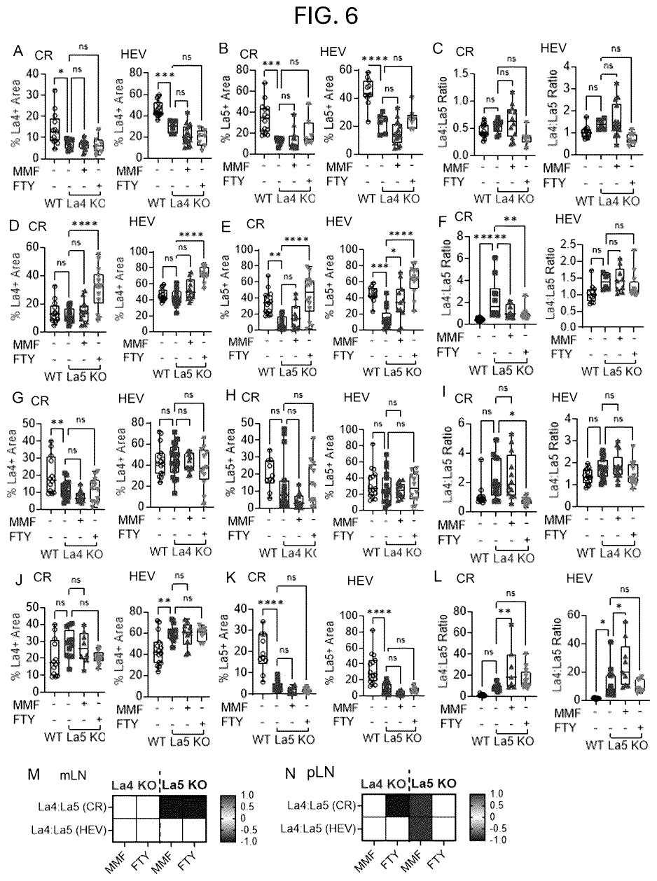

In mLNs, as we observed in WT mice, 3-day treatment of MMF or FTY did not affect laminin ratios (FIG. 3F-I, FIG. 13C, D). In FRC-Lama4-KO mice, MMF and FTY also did not affect laminin ratios (FIG. 6A-C, M). However, in FRC-Lama5-KO mice, both drugs decreased the La4:La5 in CR of mLNs (FIG. 6D-F, M). These results suggested that MMF and FTY effects on laminin composition were dependent on the FRC-derived laminin expression, indicating its critical role in maintaining the laminin balance in the mLN. The removal of FRC-derived laminin α5 appeared to unmask an early pro-inflammatory impact of MMF and FTY that was otherwise absent. This indicated that laminins specifically from FRCs acted as a protective factor in preserving stromal homeostasis in the mLNs, counteracting the early disruptive effects of these immunosuppressants.

In pLNs, as we observed in WT mice, 3-day treatment of WT mice with MMF or FTY each increased La4:La5 (FIG. 4G-J, FIG. 14C, D). MMF also increased La4:La5 in both CR and HEV of FRC-Lama5-KO mice (FIG. 6J-L, N), but not in FRC-Lama4-KO mice (FIG. 6J-L, N). In contrast, FTY decreased La4:La5 in CR of FRC-Lama4-KO mice (FIG. 6G-I, N) but not in FRC-Lama5-KO mice (FIG. 6J-L, N). These findings indicated that MMF and FTY modulated La4:La5 through distinct mechanisms that depended on the presence of laminins α4 and α5 from FRCs, although the precise pathways involved remain to be determined. The analysis of these KO models revealed that MMF relied on laminin α4 to increase laminin ratios, as its absence in FRC-Lama4-KO mice abolished the increase in La4:La5, while laminin α5 deficiency did not. Conversely, FTY depended on laminin α5 to maintain the La4:La5 ratio, as its absence revealed a decrease. These results demonstrated the distinct roles of laminins a4 and a5 in mediating immunosuppressant effects by remodeling LN architecture under MMF or FTY treatment. Collectively, these findings suggested a broader mechanistic link between immunosuppressive drugs and immune regulation, extending beyond their direct cellular effects to include the critical role of FRC-derived laminins in maintaining LN homeostasis.

### Discussion

The gut plays as a critical role in maintaining transplant tolerance by serving as a key interface between the immune system and environmental stimuli (Honda et al., Nature, (2016), 535:75-84. 10.1038/nature18848; Gabarre et al., Am J Transplant, (2022), 22:1014-1030; Salvadori et al., World J Transplant, (2024), 14:90194). In this study, we performed an in-depth characterization of the previously underappreciated, complex interplay of immunosuppressants and several gut-associated processes, including the microbiome and metabolism, intestinal gene expression, mesenteric and peripheral LN organization, and lymphocyte trafficking over time. Though not designed to target the gut, our findings revealed that immunosuppressants exert profound, time-dependent effects on the gut environment. While early treatment exhibits distinct, drug-specific effects, prolonged immunosuppressant use leads to a convergent dysbiotic state in the gut microbiome by day 30. A particularly striking observation is the convergent enrichment of the Muribaculaceae family across all four drugs after long-term treatment. These bacteria harbor a repertoire of enzymes capable of efficiently breaking down complex carbohydrates and proteins, providing them with a competitive edge in an altered gut environment under immune suppression (Ormerod et al., Microbiome, (2016), 4:36; Lagkouvardos et al., Microbiome, (2019), 7:28; Smith et al., mSphere, (2021), 6:e0085121). Their metabolic versatility likely enables them to thrive under conditions of disrupted gut homeostasis, implying that sustained immunosuppression selectively favors organisms adapted to such changes. Furthermore, recent work identifying Muribaculaceae as pathobionts capable of inducing diabetes offer a potential mechanistic link between prolonged immunosuppression and the metabolic complications often seen in transplant patients (Yang et al., Microbiome, (2023), 11:62; Kato et al., J Am Coll Cardiol, (2004), 43:1034-1041; Valantine et al., Circulation, (2001), 103:2144-2152; Biadi et al., J Heart Lung Transplant, (2007), 26:324-330). This suggests that extended immune suppression may trigger a common mechanism affecting microbial communities.

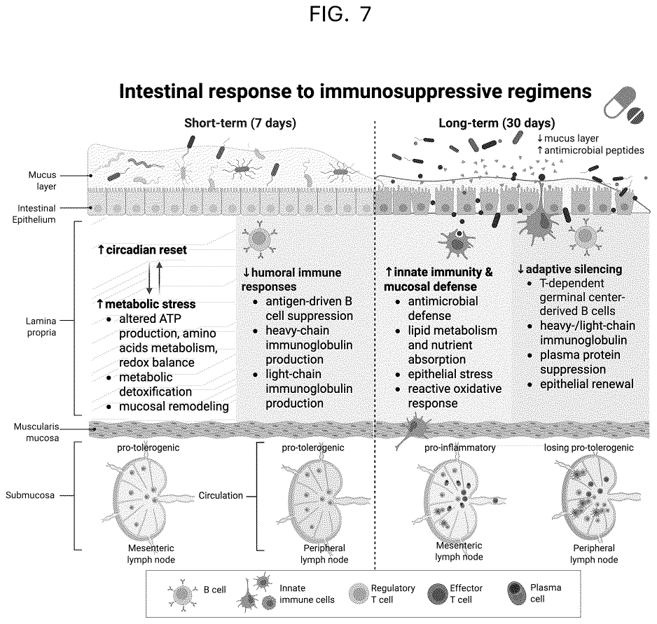

Immune suppressants also triggered a common mechanism affecting epithelial and mucosal immunity (illustrated in FIG. 7). All immunosuppressants disrupt antigen-driven B cell activation. Previous studies reported glucocorticoids directly suppress B cell function by inhibiting NF-κB-dependent transcription, leading to reduced expression of cytokines, costimulatory molecules, and survival factors essential for B cell activation and differentiation (Ashwell et al., Annu Rev Immunol, (2000), 18:309-345; Franchimont et al., Ann N Y Acad Sci, (2004), 1024:124-137). Calcineurin inhibitors impair NFAT-dependent signaling by blocking dephosphorylation of NFAT proteins, thereby inhibiting transcriptional programs involved in B cell proliferation and antibody production while also inhibiting helper T cells (Macian et al., Oncogene, (2001), 20:2476-2489). In contrast, mTOR inhibitors and anti-metabolites interfere more indirectly by suppressing T follicular helper (Tfh) cell development and function, leading to impaired T cell-dependent B cell help, germinal center formation, and class-switch recombination (Araki et al., Nature, (2009), 460:108-112). While mucosal adaptive immunity is predominately suppressed, the upregulation of a class-switched mechanism, as in Ighg2b gene activation, suggests selective survival or expansion of mucosal-associated or microbiota-reactive B cell subsets. This is supported by epithelial gene expression patterns, showing early induction of circadian resetting program and xenobiotic detoxification pathways, followed by late activation of antimicrobial peptides, ROS-generating oxidases, and stress-responsive enzymes. These changes point to a compensatory host mechanism to counteract potential pathobiont expansion and epithelial stress. However, prolonged treatments still led to transcriptomic changes in epithelial regenerative capacity, lipid metabolism, and structural remodeling. These impaired intestinal functions may predispose the mucosa to vulnerability during sustained immunosuppressive therapy. Overall, these findings reveal a complex interplay between immunosuppressive drug disturbance and the host's attempt to preserve mucosal homeostasis. Further experiments to pinpoint the precise intestinal responders common to all immune suppressants classes will be essential to understand off-target drug effects and to guide strategies to mitigate complications.

Beyond shared microbial and intestinal shifts, each immunosuppressant exhibited distinct intestinal and metabolic signatures that revealed both intended and unintended mechanistic insights. By day 30, intestinal gene expression profiles became increasingly broad, underscoring the tissue's heightened susceptibility to off-target drug effects. These differences likely arise from a combination of direct pharmacologic actions on host epithelial and immune cells, compensatory host responses, and complex interactions with the gut microbiota. At the metabolic level, each drug elicited shared and unique luminal metabolomic changes. TAC broadly altered amino acid metabolism and reduced levels of microbial-derived metabolites such as SCFAs, consistent with its known gastrointestinal side effects such as diarrhea and nonspecific abdominal discomfort (Scott et al., Drugs, (2003), 63:1247-1297). PRED exerted a widespread suppressive effect on metabolism, consistent with its ability to inhibit cellular functions and pro-inflammatory cytokines (Rhen et al., N Engl J Med, (2005), 353:1711-1723). This resulting reduction in amino acid derivatives, SCFAs, TMA, and polyamines provides insights into its side effects, including diabetes and osteoporosis (van Raalte et al., Eur J Clin Invest, (2009), 39:81-93; Compston, J., Endocrine, (2018), 61:7-16; Eastell et al., J Intern Med, (1998), 244:271-292). MMF targeted purine metabolism via IMPDH inhibition and suppressed the TCA cycle and branched-chain amino acid (BCAA) pathways, impairing ATP production and enterocyte protein synthesis. This may underlie its distinct profile of gastrointestinal toxicity and inflammation, compromised barrier integrity, and increased epithelial apoptosis and villous atrophy (Khan et al., Frontiers in physiology, (2017), 8:438; Shipkova et al., Ther Drug Monit, (2003), 25:1-16; Oellerich et al., Ther Drug Monit, (2000), 22:20-26; Helderman et al., J Am Soc Nephrol, (2002), 13:277-287). FTY, in contrast, produced the mildest metabolic disruption, primarily affecting nucleotide turnover and polyamine biosynthesis, a profile that may explain its generally better tolerability (Tedesco-Silva et al., Transplantation, (2005), 79:1553-1560).

Despite their distinct profiles, all immunosuppressants consistently affected amino acid metabolism and energy processing, suggesting a fundamental mechanism underlying long-term immunosuppression. The reduction in SCFA-producing bacteria can disrupt metabolic processes such as blood pressure regulation, lipogenesis, and blood glucose control (Bhat et al., Sci Rep, (2017), 7:10277). Studies have demonstrated that probiotics can reverse conditions like tacrolimus-induced gut dysbiosis, MMF-related intestinal toxicity and inflammation, SCFA depletion, and hypertension by restoring the vascular redox state and improving endothelial nitric oxide synthase (eNOS) function (Bhat et al., Sci Rep, (2017), 7:10277; Toral et al., Mol Nutr Food Res, (2018), 62:e1800033; Robles-Vera et al., Mol Nutr Food Res, (2020), 64:e1900616; Jardou et al., BMC Pharmacol Toxicol, (2021), 22:66). On the other hand, differences in how these drugs alter amino acid metabolism indicate that each exerts distinct influences on protein synthesis and cellular energy balance, which may explain their varying effects on different immune cell populations. Arginine metabolism, for example, is critical because it affects eNOS activity through the arginine-NO pathway, influencing NO production and ROS generation, changes that can impair endothelial function and modulate immune cell activation (Fleissner et al., Antioxid Redox Signal, (2011), 15:933-948). Both calcineurin and mTOR inhibitors drastically affect amino acid regulation, including arginine metabolism, which aligns with their documented vascular complications and supports targeted probiotic therapies to mitigate adverse metabolic and vascular outcomes (Wu et al., JCI Insight, (2025), 10:10.1172/jci.insight. 186505; Wang et al., Oncotarget, (2016), 7:53269-53276; Wang et al., Commun Biol, (2022), 5:726; Rodrigues-Diez et al., Sci Rep, (2016), 6:27915). Ultimately, the shared and drug-specific intestinal and metabolic signatures represent actionable targets for monitoring and mitigating off-target toxicity, offering new opportunities to improve safety and precision in transplantation medicine.

The impact of immunosuppressants on lymphoid tissue organization further illustrates the complex nature of their effects. Our data revealed anatomically distinct and temporally dynamic responses with lymphoid tissues. pLNs maintained an anti-inflammatory state, evidenced by elevated Tregs and increased La4:La5 ratios, particularly during early treatment phases. This response is consistent with the intended immunosuppressive action of these drugs. In contrast, mLNs, which drain the intestine, eventually developed a pro-inflammatory environment, characterized by reduced Tregs and decreased La4:La5. This compartment-specific regulatory effect may underlie some of the gastrointestinal side effects observed with immunosuppression (Ma et al., BMC Microbiol, (2023), 23:394). As illustrated in FIG. 7, these changes likely reflect feedback from the intestinal microenvironment, where metabolic stress and mucosal adaptation shape lymphoid architecture. Further insights into LN FRC-derived laminins a4 and a5 in mediating these effects suggest that stromal responses play a key role in mediating these effects (Li, L., Shirkey et al., J Clin Invest, (2020), 130:2602-2619). Notably, MMF and FTY both preserved tolerogenic LN architecture during allostimulation through the dual regulation of Tregs and stromal organization, though they were distinctly dependent on laminin α4 or laminin α5.

### Methods and Materials

### Reagents

USP grade immunosuppressants were used, when available. All drugs were prepared in a manner approved by the University of Maryland School of Medicine IACUC: TAC (USP grade, MilliporeSigma, Burlington, MA) was reconstituted in DMSO (USP grade, MilliporeSigma) at 20 mg/ml for storage. At time of use, TAC/DMSO stock was diluted with absolute ethanol (USP grade, Decon Labs, King of Prussia, PA) to 1.5 mg/ml then further diluted 1:5 in sterile PBS and injected at 10 μl/gm s.c. (3 mg/kg/day) (Bromberg, et al., JCI Insight, (2018), 3:10.1172/jci.insight.121045). PRED (MilliporeSigma) was reconstituted in PBS at 50 mg/ml for storage and then diluted to 0.5 mg/ml in PBS and injected at 5 mg/kg/di.p. (Chin et al., J Am Soc Nephrol, (2021), 32:199-210). MMF (MilliporeSigma) was reconstituted at 30 mg/ml in DMSO for storage and then diluted in PBS to 1.5 mg/ml and at 30 mg/kg/d i.p. (Richez et al., PLOS One, (2013), 8:e61042). FTY (Fingolimod, MilliporeSigma) was reconstituted at 3 mg/ml in 1:1 PBS:Ethanol for storage and then diluted 1:10 in PBS and administered at 3 mg/kg/d p.o. (Honig et al., Journal of Clinical Investigation, (2003), 111:627-637). The control group received PBS only.

### Mice Experiments

Mouse experiments were performed according to ARRIVE guidelines. 8- to 14-week-old female C57BL/6 mice were purchased from The Jackson Laboratory (Bar Harbor, ME, USA). Mice were maintained at the University of Maryland School of Medicine Veterinary Resources animal facility. Only female mice were used to ensure a high degree of homogeneity within our study groups. The Pdgfrb-Cre+/−×La4fl/fl and Pdgfrb-Cre+/−×La5fl/fl conditional knockout (KO) mice were previously developed in our laboratory (Li, L., Shirkey et al., J Clin Invest, (2020), 130:2602-2619; Li et al., J Clin Invest, (2022), 132:10.1172/JCI156994). All mice were cohoused for a minimum of 2 weeks prior to experiments to normalize microbiota. During this period, mice from different treatment groups were housed in the same room and adjacent cages under identical conditions to ensure normalized environmental exposure. At experimental day 0, the mice were randomly separated to different experimental groups, and then kept groups in separate cages to prevent cross exposure. Mice received daily immunosuppression following the dosages in Table 6. In allogeneic stimulation experiment, mice received 10\hat{ }7 fully allogeneic BALB/c splenocytes intravenously (i.v.) on day 0 followed by drug treatment. On the day of harvest, the mice were euthanized by CO₂ narcosis, intraluminal stool samples collected for metabolomic and microbiome analyses, cardiac puncture utilized for blood collection, and mesenteric and peripheral (axillary, inguinal, and brachial) LNs and portions of the small intestine between the duodenum and jejunum harvested for immunological assays. All procedures involving mice were performed in accordance with the guidelines and regulations set by the Office of Animal Welfare Assurance of the University of Maryland School of Medicine.

TABLE 6
Immunosuppression drugs and dosage.

| Treatment Groups | Dose*(mg/kg/day) | Source (catalog #) | Route |
|---|---|---|---|
| Tacrolimus | 3 mg/kg/day | Sigma Aldrich | Subcutaneous |
| | | (Y0001926) | |
| Prednisolone | 5 mg/kg/day | Sigma Aldrich | Intraperitoneal |
| | | (P6004) | |
| Mycophenolate mofetil | 30 mg/kg/day | Sigma Aldrich | Intraperitoneal |
| | | (SML0284) | |
| FTY | 3 mg/kg/day | Sigma Aldrich | Oral |
| | | (SML0700) | |

### Stool Specimen Collection, DNA Extraction, and Metagenomic Sequencing

As described previously, stool samples were collected and stored immediately in DNA/RNA Shields (Zymo Research, Irvine, CA, USA) at −80° C. (Wu et al., JCI Insight, (2025), 10:10.1172/jci.insight. 186505; Ma et al., BMC Microbiol, (2023), 23:394; Bromberg et al., JCI Insight, (2018), 3:10.1172/jci. insight. 121045; Ma et al., Sci Rep, (2023), 13:1023). This stabilizes and protects the integrity of nucleic acids and minimizes the need for immediate processing of specimens. The Quick-DNA Fecal/Soil Microbe kit (Zymo Research, Irvine, CA, USA) was used to extract DNA from 0.15-0.25 grams of fecal samples. To ensure that no exogenous DNA contaminated the samples, we included negative extraction controls. Construction of the metagenomic sequencing libraries was performed as previously (Wu et al., JCI Insight, (2025), 10:10.1172/jci.insight. 186505; Ma et al., BMC Microbiol, (2023), 23:394; Ma et al., Sci Rep, (2023), 13:1023). In brief, the Nextera XT Flex Kit (Illumina), according to the manufacturer's recommendations, libraries were then pooled together in equimolar proportions before they were sequenced on a single Illumina NovaSeq 6000 S2 flow cell at Maryland Genomics at the University of Maryland School of Medicine. 92.7±8.5 (mean±s.e.) million reads per sample were obtained.

### Gut Microbiome Analyses

Sequence analyses were performed as previously described (Wu et al., JCI Insight, (2025), 10:10.1172/jci.insight. 186505; Ma et al., BMC Microbiol, (2023), 23:394; Ma et al., Sci Rep, (2023), 13:1023; Bromberg et al., JCI Insight, (2018), 3:10.1172/jci. insight. 121045). Metagenomic sequence reads mapping to Genome Reference Consortium Mouse Build 39 of strain C57BL/6J (GRCm39) were removed using BMTagger v3.101 (Rotmistrovsky et al., (Integrative Human Microbiome Project (iHMP) by NCBI/NLM, National Institutes of Health), (2011). Sequence read pairs were removed when one or both the read pairs matched the genome reference. The Illumina adapter was trimmed and quality assessment was performed using default parameters in fastp (v.0.21.0) (Chen et al., Bioinformatics, (2018), 34:1884-1890). The taxonomic composition of the microbiomes was established using Kraken2 (v.2020.12) and Braken (v. 2.5.0) using the comprehensive mouse microbiota genome catalog (Kieser et al., PLOS Comput Biol, (2022), 18:e1009947). Phyloseq R package (v1.38.0) was used to generate the barplot and diversity index. Linear discriminant analysis (LDA) effect size (LEfSe) analysis was used to identify fecal phylotypes that could explain the differences. The α value for the non-parametric factorial Kruskal-Wallis (KW) sum-rank test was set at 0.05 and the threshold for the logarithmic LDA model score for discriminative features was set at 2.0. An all-against-all BLAST search was performed in the multiclass analysis. Taxonomic ordination graphs were created with the micro Viz (v0.12.4) (D. J., et al., J Open Source Softw, (2021), 6:3201). The metagenomic dataset was mapped to the protein database UniRef90 to ensure the comprehensive coverage in functional annotation, and was then summarized using HUMAnN3 (Human Microbiome Project Unified Metabolic Analysis Network) (v0.11.2) to determine the presence, absence, and abundance of metabolic pathways in a microbial community. MetaCyc pathway definitions and MinPath were used to identify a parsimonious set of pathways summarized in HUMAnN3 in the microbial community. Canonical Correspondence Analysis (CCA) was performed using the vegan package based on the Bray-Curtis distance. Based on their eigenvalues, CA1 and CA2 were selected as the major components (Jari Oksanen et al., R package, (2016); Dixon et al., Journal of Vegetation Science, (2003), 14, 927-930). To evaluate microbiome profile differences across groups and time points, the line plot was generated using the average pairwise Bray-Curtis distances between all group pairs at each time point.

### Metabolite Extraction and Metabolome Analyses

As previously described, capillary electrophoresis-mass spectrometry (CE/MS) was used for measuring metabolome of intraluminal stool (luminal/local) to obtain a comprehensive quantitative survey of metabolites (Human Metabolome Technologies, Boston, MA, USA) for immunosuppressants TAC, PRED, MMF, and a no-treatment control group processed concurrently (Wu et al., JCI Insight, (2025), 10:10.1172/jci.insight.186505; Ma et al., BMC Microbiol, (2023), 23:394; Bromberg et al., JCI Insight, (2018), 3:10.1172/jci. insight. 121045; Ma et al., Sci Rep, (2023), 13:1023). For FTY, stool pellets were collected at the same time points for metabolomic analysis due to technical limitations in obtaining sufficient sample volumes for comprehensive profiling. The no-treatment control for FTY also used stool pellets and processed concurrently. All subsequent analyses were performed using the respective no-treatment control groups and the same specimen types, processed concurrently with their corresponding experimental groups, to ensure valid comparisons. ~10-30 mg of stool was weighed at the time of collection using a company-provided vial and stored at −80° C. until shipped to vendor on dry ice. QC procedures included standards, sample blanks and internal controls that were evenly spaced among the samples analyzed. Compound identification was performed using a CE/MS library of >1,600 annotated molecules.

To reduce the influence of measurement noise, rigorous data pretreatment was performed according to validated procedures and are as previously described (van den Berg et al., BMC Genomics, (2006), 7:142; Wu et al., JCI Insight, (2025), 10:10.1172/jci.insight.186505; Ma et al., BMC Microbiol, (2023), 23:394; Ma et al., Sci Rep, (2023), 13:1023). Metabolites were annotated using PubChem, KEGG, and HMDB annotation frameworks that leverage cataloged chemical compounds, known metabolic characterization, and functional hierarchy (i.e., reaction, modules, pathways) (Kim et al., Nucleic Acids Res, (2021), 49:D1388-D1395; Hattori et al., Nucleic Acids Res, (2010), 38:W652-656; Wishart et al., Nucleic Acids Res, (2018), 46:D608-D617). Partial least squares discriminant analysis (PLS-DA) implemented using mixOmics (vers. 6.18.1) was used (Le Cao et al., BMC Bioinformatics, (2011), 12:253). The “sparseness” of the model was adjusted by the number of components in the model and the number of variables within each component based on the classification error rate with respect to the number of selected variables. Tuning was performed one component at a time, and the optimal number of variables to select was calculated. The volcano plot combines results from fold change (FC) analysis to show significantly increased metabolites after 7-day tacrolimus treatment. A metabolite is shown if FC is >2 and the p-value is <0.05 based on 2-sample t-tests. Original metabolite measurements without normalization were used in the FC analysis. For the metabolites annotated in a specific functional pathway, metabolite set enrichment analysis (MSEA) was performed (Xia et al., Nucleic Acids Res, (2010), 38:W71-77).

### RNA Isolation, Transcriptome Sequencing, and Bioinformatics Analyses

RNA isolation, transcriptome sequencing, and bioinformatics analyses are as previously described (Wu et al., JCI Insight, (2025), 10:10.1172/jci.insight. 186505; Ma et al., BMC Microbiol, (2023), 23:394; Ma et al., Sci Rep, (2023), 13:1023). Dissected intestinal tissues were stored immediately in RNAlater solution (QIAGEN) in RNAse-free tubes (Denville Scientific, Holliston, MA) to stabilize and protect the integrity of RNA (Gorokhova et al., Limnol. Oceanogr.: Methods, (2005), 3:143-148). Specimens were stored at −80° C. until extraction. For each sample, total RNA was extracted from ~1 cm of ileum. Prior to the extraction, 500 μl of ice-cold RNase free PBS was added to the sample. To remove the RNAlater, the mixture was centrifuged at 8,000×g for 10 min and the resulting pellet resuspended in 500 μl ice-cold RNase-free PBS with 10 μl of β-mercaptoethanol. A tissue suspension was obtained by bead beating procedure using the FastPrep lysing matrix B protocol (MP Biomedicals, Solon, OH) to homogenized tissues. RNA was extracted from the resulting suspension using TRIzol Reagent (Invitrogen, Carlsbad, CA) following the manufacturer's recommendations and followed by protein cleanup using Phasemaker tubes (Invitrogen) and precipitation of total nucleic acids using isopropanol. RNA was resuspended in DEPC-treated DNAase/RNAase-free water. Residual DNA was purged from total RNA extract by treating once with TURBO DNase (Ambion, Austin, TX, Cat. No. AM1907) according to the manufacturer's protocol. DNA removal was confirmed via PCR assay using 16S IRNA primer 27F (5′-AGAGTTTGATCCTGGCTCAG-3′) (SEQ ID NO:1) and 534R (5′-CATTACCGCGGCTGCTGG-3′) (SEQ ID NO:2). The quality of extracted RNA was verified using the Agilent 2100 Expert Bioanalyzer using the RNA 1000 Nano kit (Agilent Technologies, Santa Clara, CA). Ribosomal RNA depletion and library construction were performed using the RiboZero Plus kit and TruSeq stranded mRNA library preparation kit (Illumina) according to the manufacturer's recommendations. Libraries were then pooled together in equimolar proportions and sequenced on a single Illumina NovaSeq 6000 S2 flow cell at the Genomic Resource Center (Institute for Genome Sciences, University of Maryland School of Medicine) using the 150 bp paired-end protocol. 143, 2±53.4 (mean±s.e.) million reads per sample were obtained. The quality of sequencing was evaluated by using FastQC (Andrews, S. FastQC: A Quality Control Tool for High Throughput Sequence Data, (2010). Reads were aligned to the mouse genome (Mus_musculus.GRCm39) using HiSat (version HISAT2-2.0.4) and the number of reads that aligned to the coding regions was determined using HTSeq (Kim et al., Nat Methods, (2015), 12:357-360; Anders et al., Bioinformatics, (2015), 31:166-169). Significant differential expression was assessed using DESeq2 with an FDR value≤0.05 and FC>2 (Anders et al., Genome Biol, (2010), 11: R106). The over-representative analysis was done by importing Differentially Expressed Genes (DEGs) against GO ontologies using the enrichGO function of clusterProfile Bioconductor package (G. Yu et al., OMICS: A Journal of Integrative Biology, (2012), 16:284-287). Only the ontology terms with q value <0.05 were used for plotting. The normalized enrichment score was calculated as the ratio of the proportion of DEGs annotated to a given GO term to the proportion of genes in the reference annotated to the same GO term. UpSet plots were generated using UpSetR (vers. 1.4.0) to analyze overlapping DEGs between days 7 and 30 and across different treatments.

### Flow Cytometry

The procedure is as previously described (Wu et al., JCI Insight, (2025), 10:10.1172/jci.insight.186505; Ma et al., BMC Microbiol, (2023), 23:394). To produce single-cell suspensions, LNs were disaggregated and passed through 70-μm nylon mesh screens (Thermo Fisher Scientific, Waltham, MA). Antibodies against surface molecules were incubated with cell suspensions for 30 min at 4° C. (Table 7) then washed with FACS buffer [PBS with 0.5% w/v bovine serum albumin] twice. Cells were then permeabilized, according to manufacturer's protocol, with Foxp3/Transcription Factor Staining Buffer Set (eBioscience, San Diego, CA), washed with FACS buffer, and subsequently stained with antibodies for intracellular molecules at 4° C. Samples were analyzed with an LSR Fortessa Cell Analyzer (BD Biosciences), and data were analyzed using FlowJo software version 10.6 (BD Biosciences). Single color controls (cells stained with single surface marker antibody) and unstained controls were used for flow channel compensation.

TABLE 7
List of Antibodies

| Target Molecule | Clone | Catalog No. |
|---|---|---|
| Anti-Hamster IgG | Polyclonal | Jackson ImmunoResearch; 127-165-160 |
| Cy3 | | |
| Anti-Mouse IgG- | Polyclonal | Jackson ImmunoResearch; 715-605-151 |
| AF647 | | |
| Anti-Rabbit AF488 | Polyclonal | Jackson ImmunoResearch; 111-545-003 |
| Anti-Rabbit Cy5 | Polyclonal | Jackson ImmunoResearch; 111-175-003 |
| Anti-Rabbit DL405 | Polyclonal | Jackson ImmunoResearch; 711-476-152 |
| Anti-Rabbit IgG- | Polyclonal | Jackson ImmunoResearch; 711-545-152 |
| AF488 | | |
| Anti-Rabbit IgG- | Polyclonal | Jackson ImmunoResearch; 111-585-003 |
| AF594 | | |
| Anti-Rabbit IgG- | Polyclonal | Jackson ImmunoResearch; 711-606-152 |
| AF647 | | |
| Anti-Rat IgG AF594 | Polyclonal | Jackson ImmunoResearch; 112-586-143 |
| Anti-rat IgG AF647 | Polyclonal | Jackson ImmunoResearch; 712-606-153 |
| CD4 | GK1.5 | Biolegend; 100401 |
| B220 | RA3-6B2 | eBioSci; 17-0452-82 |
| CD11b | M1/70 | eBioSci; 11-0112-81, 17-0112-82 |
| CD8 | 53-6.7 | Biolegend; 100701 |
| CD11c | HL3 | BD; 550283, 557401 |
| ER-TR7 | ER-TR7 | Novus; NB100-64932 |
| ER-TR7 | Polyclonal | Santa Cruz; SC-73355 |
| Foxp3 | NRRF-30 | eBioSci; 14477180 |
| Foxp3 | FJK-16s | eBioSci; 12-5773-82 |
| Laminin α4 | 775830 | R&D; MAB3837 |
| Laminin α5 | Polyclonal | Novus Biol; NBP1-18714 |

### Immunohistochemistry

We excised mLN, pLN and segments of the intestine between the duodenum and jejunum then immediately submerged in OCT compound (Scigen Paramount, CA, USA). Cryosections (6-7 μm for LNs, 10 μm for intestine) were cut in duplicate or triplicate depending on size of block using a Microm HM 550 cryostat (ThermoFisher Scientific). Slides were then fixed in cold 1:1 acetone: methanol for 5 min, air dried for 15 min, and stored at −80° C. until use. At the time of staining, slides were thawed for 15 minutes at room temperature then sections were rehydrated in PBS and blocked with 10% secondary antibody host serum PBST (PBS+0.03% Triton-X-100+0.5% BSA). The slides were then stained for 1 hour with primary antibodies at room temperature (diluted 1:50-1:200 in PBST) for 1 hour incubated with secondary antibodies (diluted 1:100-1:400 in PBST) for 1 hour. After staining, slides were fixed with 4% paraformaldehyde in PBS for 5 min, quenched with 1% glycerol in PBS for 5 min, and mounted with Prolong Gold Antifade Mountant with or without DAPI (Thermo Fisher Scientific). Images were acquired using Accu-Scope EXC-500/Nikon (Accu-Scope, Commack, NY, and Nikon, Tokyo, Japan) and analyzed using Volocity software (Quorum Technologies Inc., Sacramento, CA). The antibodies used are listed in Table 7. For each mouse, 1-2 mLNs and 2-3 pLNs, and 1 piece of intestine were collected. All samples from each treatment group were combined and analyzed as a block, 2-3 sections/block were placed on slides, and 7-30 fields/slide were analyzed. The average of MFI for each treatment group was calculated by averaging the MFI values across all slides from all mice, within demarcated HEVs and CR regions of mLN and pLN and whole intestinal images. Qualitative heat maps were generated (GraphPad prism) to express changes in IHC marker expression level relative to control using-1 to represent “decreased” (blue in the heatmap), 0 to represent “unchanged” (white in the heatmap), and 1 to represent “increased” (red in the heatmap). GraphPad Prism 10.3.1 (San Diego, CA, USA) was used for analysis with statistical significance defined as P<0.05. Tukey's multiple comparison tests of one-way ANOVA were used for comparisons of fluorescence.

### Example 2. Pro-tolerogenic Bifidobacterium pseudolongum-Driven Remodeling of the Post-Transplant Gut Ecosystem Counters Immunosuppressant-Induced Dysbiosis

Chronic allograft vasculopathy remains the chief cause of late cardiac graft loss and is failed to be controlled by standard immunosuppressant regimens. We previously isolated a protolerogenic gut commensal Bifidobacterium pseudolongum subsp globosum (Bifido), which significantly prolonged graft acceptance and dampened both systemic and intragraft inflammatory responses. We recently demonstrated that five mechanistically distinct immunosuppressant drug classes converge on a shared dysbiotic gut state marked by loss of microbial diversity and robust expansion of Muribaculaceae, accompanied by coordinated metabolic perturbations involving amino acid utilization, nucleotide and purine metabolism, bile acid signaling, and microbial-derived inflammatory metabolites.

In this example, we employed a fully allogeneic murine heart transplant model maintained on daily tacrolimus to longitudinally interrogate gut microbiome dynamics under immunosuppression. Tacrolimus alone drove a progressive shift toward Muribaculaceae dominance over time. In contrast, oral administration of the Bifido imposed an early (day 7) and sustained divergence from both tacrolimus-only controls and Desulfovibrio (Desulfo) treated mice, a pro-inflammatory control. Bifido treatment attenuated Muribaculaceae expansion (including Duncaniella and CAG-873), enriched fermentative commensals, and was associated with minimal graft vasculitis and reduced fibrosis. By contrast, Desulfo and tacrolimus-only controls, particularly Desulfo exhibited progressive vascular inflammation, which became severe by day 40, along with significantly increased graft fibrosis despite systemic immunosuppression.

Human-sourced fecal microbiota transfer (FMT) recapitulated these ecological rules. FMT from heart transplant patients with cardiac allograft vasculopathy (CAV) promoted expansion of endogenous murine Muribaculaceae and other immunogenic taxa, whereas non-CAV showed colonization by B. longum and B. pseudolongum, and relatively more short chain fatty acid (SCFA) producers. Overall, our study showed a potent protolerogenic commensal, B. pseudolongum, reprograms the tacrolimus-treated gut ecosystem, suppressing dysbiosis marked by Muribaculaceae, and preserving graft vascular integrity. In this model, the targetable feature of gut dysbiosis is the emergence of a Muribaculaceae-dominant community state (e.g., Duncaniella/CAG-873), which is reproducible under tacrolimus, associates with vascular injury, and is reversible by Bifido. These data support microbiome-targeted adjuncts to complement immunosuppression and mitigate immune-mediated graft rejection.

Gut Microbial Features as the “Targetable” after Cardiac Transplantation

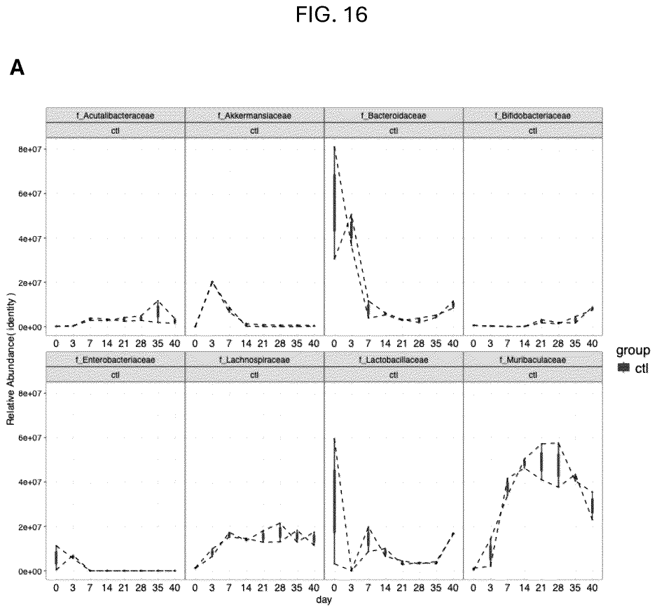

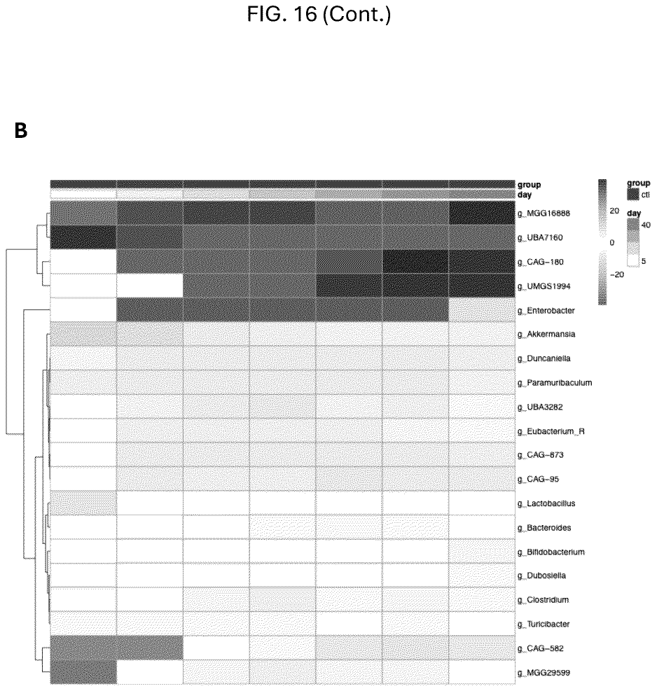

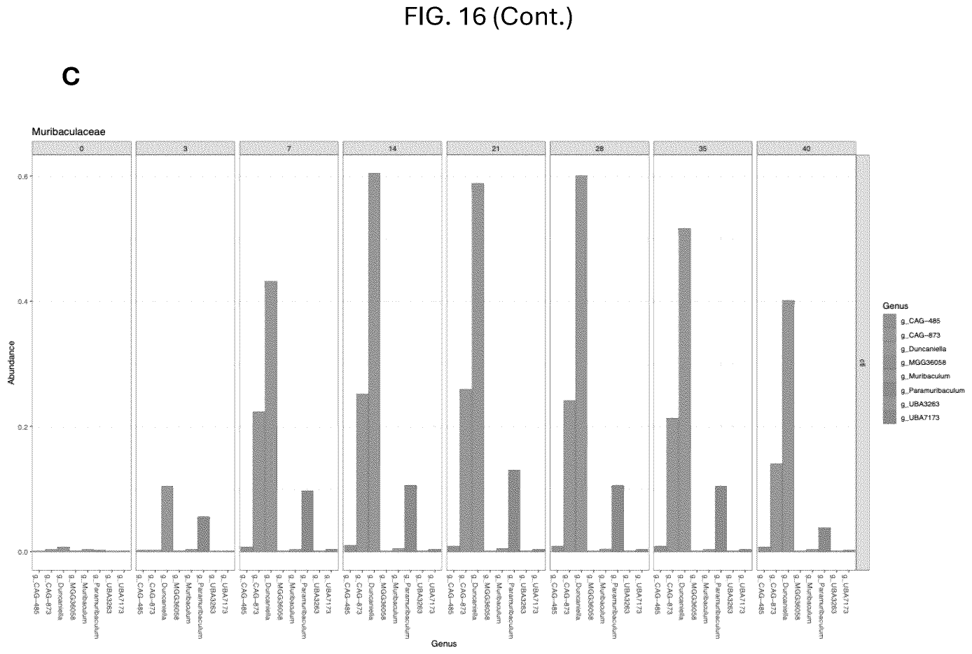

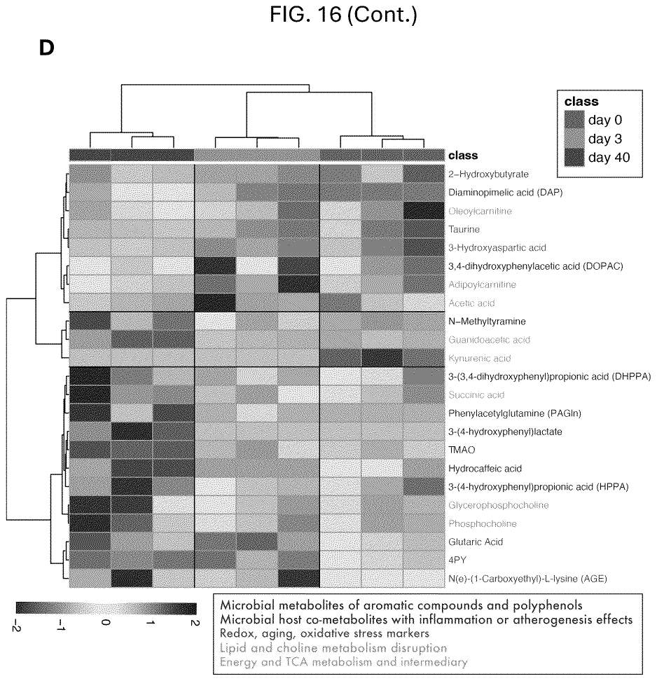

We previously demonstrated five mechanistically distinct drug classes induced a conserved dysbiotic state marked by reduced diversity and expansion of Muribaculaceae. In this study, we thus examined gut microbiome longitudinal changes in our cardiac transplant model in which tacrolimus is administered daily after allografting. Longitudinal profiling revealed distinct, time-structured shifts of the gut ecosystem over 40 days. (FIG. 16A). These changes clustered into three major categories: (i) families undergoing early depletion, (ii) families with transient early expansion, and (iii) families showing late or sustained enrichment. Specifically, several commensal families and genera were rapidly reduced within the first two weeks and remained at low abundance throughout the study (FIG. 16B). Akkermansiaceae (Akkermansia muciniphila) declined rapidly, becoming nearly undetectable by day 14. Bacteroidaceae (Bacteroides thetaiotaomicron) also dropped by day 7 and remained low thereafter. Likewise, Enterobacteriaceae decreased to near-zero levels by day 7, and Lactobacilluseae fell sharply by day 3. These taxa include barrier-associated and mucin-degrading species, and their reduction at early stages suggest inflammatory risk due to chronic immunosuppression. In contrast, members of the Lachnospiraceae (14-2, Acetatifactor, 1XD42-69, 1XD8-76) exhibited a rapid expansion peaking at day 7, indicating a transient fermentative response following loss of primary mucin- and polysaccharide-utilizing taxa. Minor families such as Acutalibacteraceae also showed modest early increases by day 7 but failed to persist.

Muribaculaceae expanded most prominently, peaking near day 14 and remaining the dominant family through day 40, with a mild decline after day 28 (FIG. 16C). Within this family, Duncaniella, CAG-873, and Paramuribaculum were the principal contributors to the expansion. Notably, all three genera in this Muribaculaceae expansion correspond to the convergent dysbiosis previously observed in naïve mice exposed to diverse immunosuppressants. By day 21, Bifidobacteriaceae (Bifidobacterium pseudolongum) appeared and persisted through day 40. Because B. pseudolongum is an endogenous, protolerogenic murine strain, its delayed appearance may suggest its adaptation to the immune suppressed environment. Overall, tacrolimus profoundly reshaped the post-transplant microbiota in a staged manner. The 40-day treatment resulted in a gut microbiome closely recapitulating the convergent dysbiosis seen under chronic immunosuppression in naïve mice, suggesting the microbial changes are primarily due to the immunosuppressants treatment than transplant.

We longitudinally profiled the ileal intraluminal metabolome in the cardiac allograft model receiving daily tacrolimus (FIG. 16D). Consistent with the microbiome results, the gut metabolic activities also underwent time-structured shifts over 40 days. At day 0, immediately after antibiotic conditioning and before transplantation and tacrolimus onset, the lumen was enriched for N-methyltyramine, guanidinoacetic acid, and kynurenic acid, which are reduced by day 3 and almost totally depleted by day 40. By day 3, the profile shifted toward markers of redox and membrane-energy stress with 2-hydroxybutyrate, oleoylcarnitine and adipoylcarnitine (fatty-acid oxidation flux), and taurine (bile-acid conjugation/sulfur pool). In parallel, diaminopimelic acid (DAP), a peptidoglycan intermediate, rose, consistent with heightened bacterial cell-wall turnover during community restructuring. Additional increases in 3-hydroxyaspartic acid, DOPAC (3,4-dihydroxyphenylacetic acid), and acetic acid marked early host-microbe metabolic engagement. The day 0-3 window captures an acute injury/IS-conditioning phase: oxidative and mitochondrial stress (2-hydroxybutyrate; acylcarnitines), bile-acid/taurine handling, and accelerated microbial growth/lysis (DAP). These changes align with early immune activation and barrier perturbation immediately post-Txp under tacrolimus.

By day 40, a distinct steady-state emerged characterized by microbiota-derived aromatic and choline-axis metabolites: 3-(3,4-dihydroxyphenyl) propionic acid (DHPPA), 3-(4-hydroxyphenyl) propionic acid (HPPA), 3-(4-hydroxyphenyl) lactate, hydrocaffeic acid, phenylacetylglutamine (PAGIn), and trimethylamine N-oxide (TMAO), together with succinic acid, glycerophosphocholine, phosphocholine, glutaric acid, 4PY (N-methyl-4-pyridone-3-carboxamide), and Nε-(1-carboxyethyl)-L-lysine (AGE). HPPA/HPPA/phenyl-lactate/hydrocaffeic acid indicate sustained microbial metabolism of phenylalanine/tyrosine and dietary polyphenols, consistent with a remodeled saccharolytic community that increasingly taps proteo-/aromato-genic substrates. Succinate accumulation aligns with Bacteroidales-style fermentation and the observed Muribaculaceae dominance; luminal succinate can signal via SUCNR1 and support inflammatory tone. Glycerophosphocholine/phosphocholine together with TMAO implicate activated choline/TMA metabolism and hepatic oxidation to TMAO, pathways linked to vascular inflammation and platelet reactivity. 4PY suggests heightened NAD turnover; AGE reflects carbonyl stress/glycation, compatible with chronic low-grade inflammation under immunosuppression. PAGln further supports microbe-host cometabolism of phenylalanine with potential cardiovascular activity. In summary, early acute oxidative/mitochondrial stress, later a remodeled, Muribaculaceae-dominant community marked by succinate accumulation and activation of aromatic-amino-acid and choline-TMA cometabolism, with systemic-facing signatures (TMAO, PAGln, AGE, 4PY) indicative of endothelial/vascular stress potential.

Bifidobacterium reshapes the post-transplant microbiome by attenuating Muribaculaceae dominance and enriching fermentative commensals

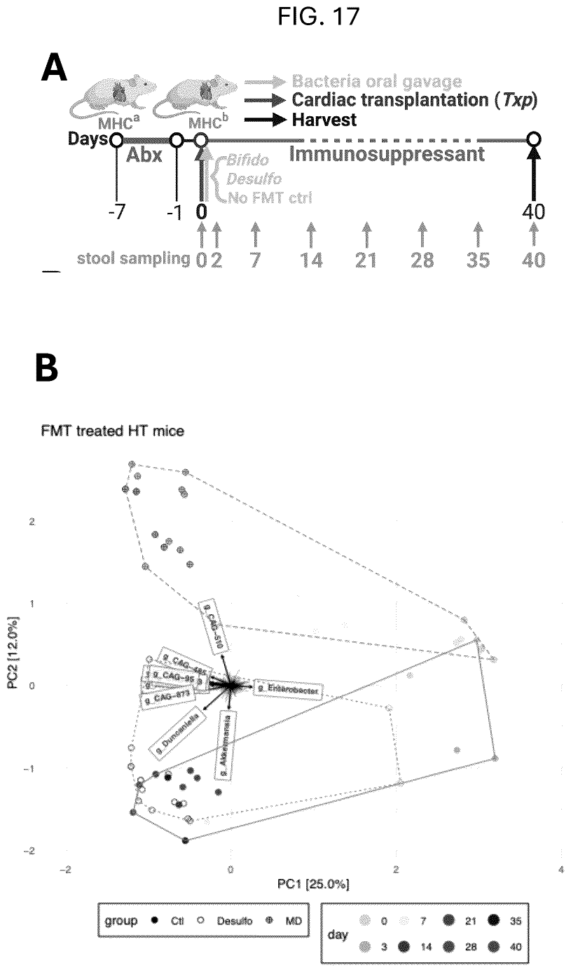

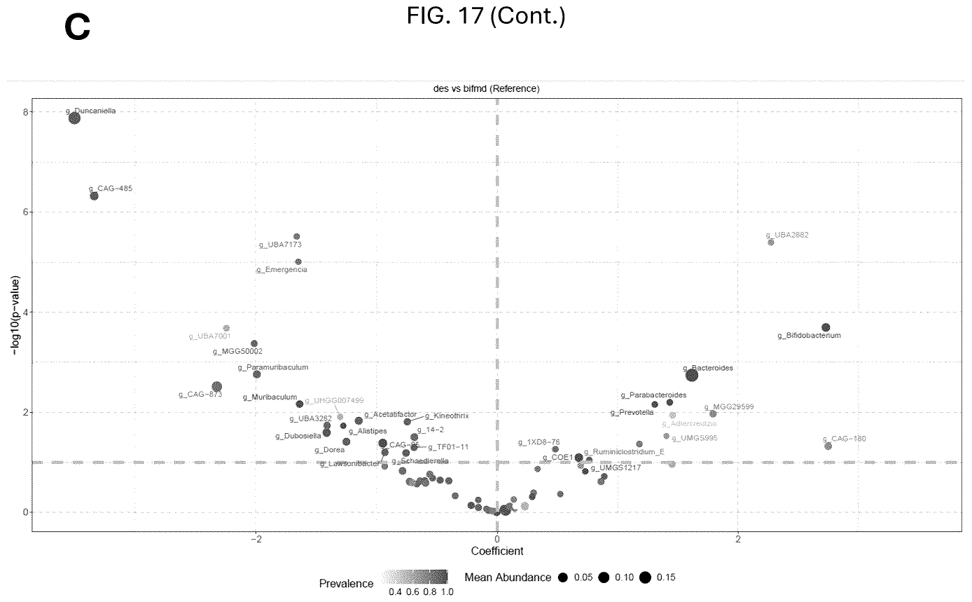

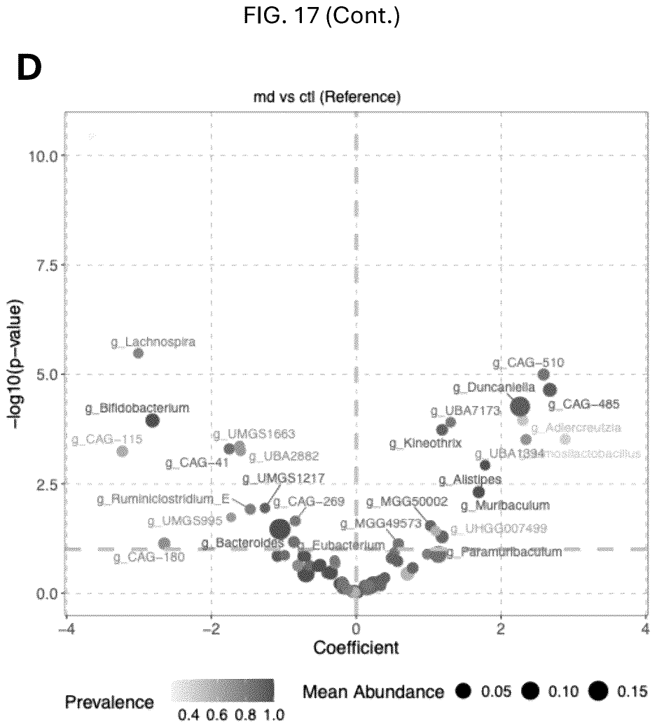

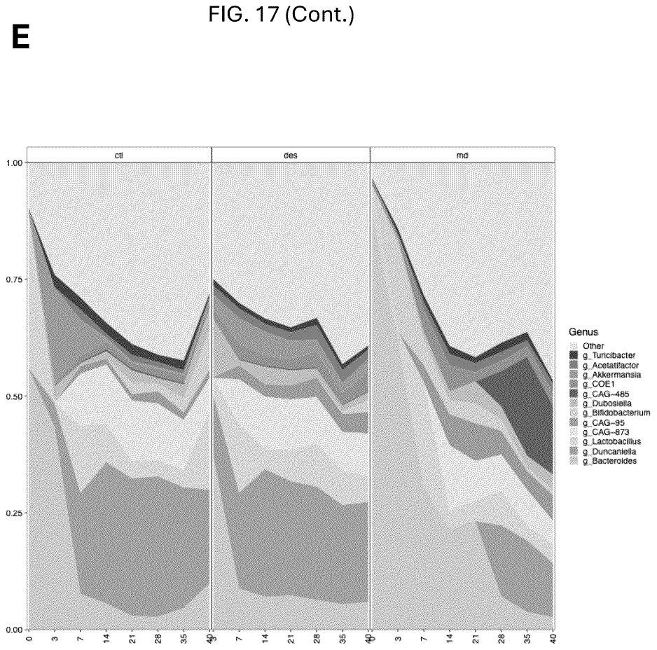

To determine whether the introduction of a single protolerogenic bacterium can alter the post-transplant gut ecosystem, we gavaged cardiac allograft recipients with Bifidobacterium pseudolongum (Bifido), Desulfovibrio desulfuricans (Desulfo), or PBS (tacrolimus-only control) (experimental design in FIG. 17A). Principal component analysis demonstrated that by day 40, the Bifido group diverged most markedly from Desulfo and control groups, and the changes became more apparent over time (FIG. 17B). Taxa driving this separation were dominated by Muribaculaceae members, indicating that Bifido altered the ecological balance of a family previously shown to expand under chronic immunosuppression. Alpha diversity (Shannon index) did not differ significantly among groups, although Bifido-treated mice exhibited a steeper diversity increase over time compared with Desulfo and control cohorts.

To systematically assess temporal trends, we employed mixed-effects models incorporating time, group, and subject effects. This analysis revealed profound group-specific enrichments (FIG. 17C). Bifido gavage enriched not only for Bifidobacterium itself, but also taxa from diverse families including CAG-180 (Oscillospirales), UBA2882 (Lachnospiraceae), Bacteroides and Prevotella (Bacteroidaceae), Ruminiclostridium (Ruminococcaceae), and COE1 and 1XD8-76 (Lachnospiraceae). In contrast, the Desulfo group was enriched for multiple Muribaculaceae lineages (Duncaniella, CAG-485, UBA7173, Paramuribaculum, Muribaculum, CAG-873), as well as Emergencia (Peptostreptococcales), MGG50002 (Lachnospiraceae), and uba7001 (TANB77). This comparison yielded the greatest number of significantly differentially abundant taxa, underscoring the broad reprogramming imposed by Bifido.

Relative to tacrolimus-only controls, Bifido treatment reduced enrichment of canonical Muribaculaceae members (FIG. 17D) (Duncaniella, CAG-485, UBA7173, CAG-873) and selected fermenters (CAG-510, Kineothrix, UBA1394), while increasing multiple lineages across Bacteroidaceae, Lachnospiraceae, and Ruminococcaceae. By contrast, Desulfo and control groups exhibited only modest differences, with both enriched in overlapping Muribaculaceae clades. These findings indicate that Bifido reshapes the gut ecosystem more profoundly than either Desulfo or tacrolimus alone (FIG. 17E). Mechanistically, Bifido attenuated the dominance of Muribaculaceae, a signature of immunosuppressant-induced dysbiosis, and instead promoted expansion of diverse fermentative commensals and mucosa-associated taxa (Bacteroides, Prevotella, Lachnospiraceae, Ruminococcaceae). This shift was consistent with prior work showing that B. pseudolongum enhances immune tolerance via remodeling of local gut ecology and restoring microbial metabolic capacity. Together, these results demonstrated that pro-tolerogenic Bifido imposed sustained and functionally significant remodeling of the endogenous microbiome in transplant recipients, counteracting tacrolimus-associated Muribaculaceae expansion and enriching for commensals with fermentative and immunomodulatory potential.

Bifido induces dynamic changes in the endogenous gut microbiome and preserves cardiac graft vascular integrity

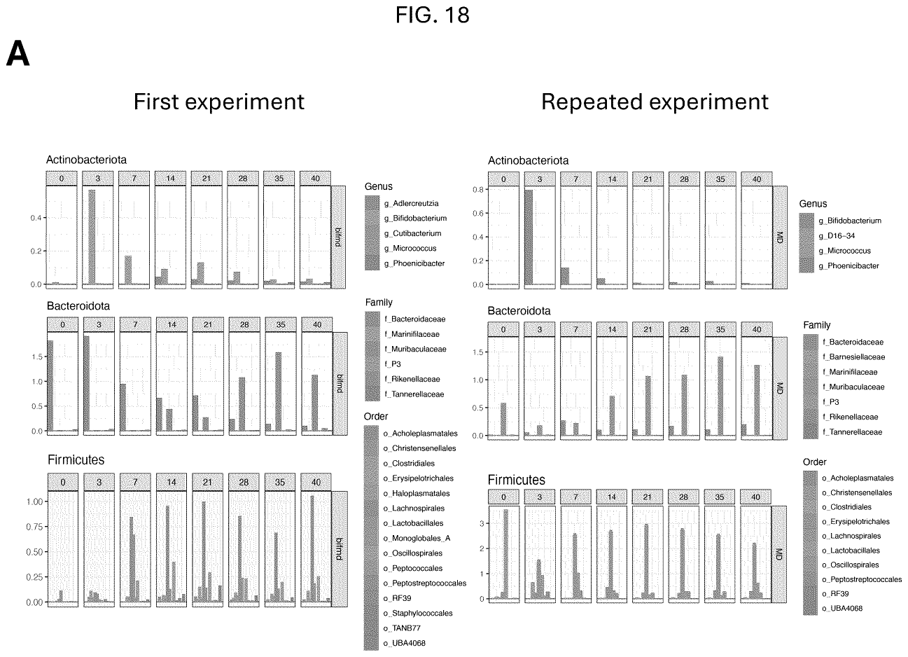

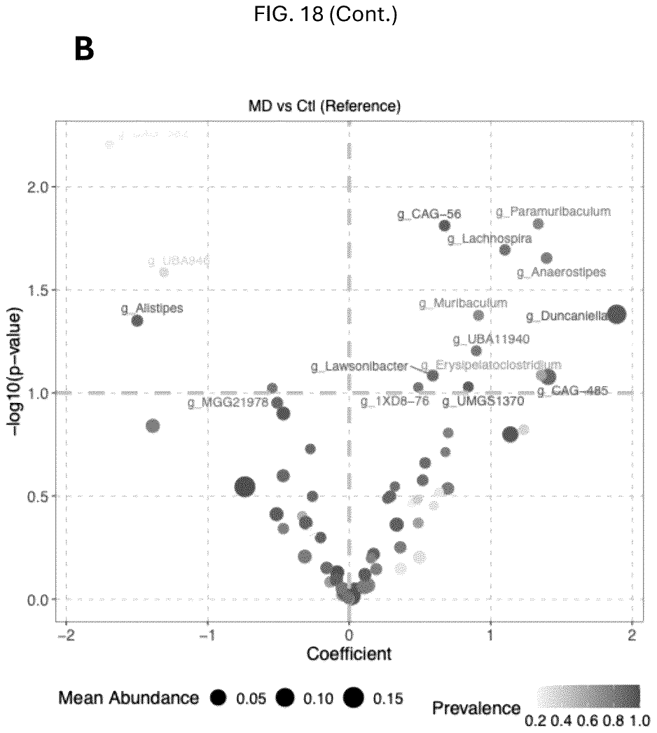

To assess the temporal impact of Bifido on the endogenous gut microbiome after cardiac transplantation, we performed trajectory analysis using PCoA connected by temporal vectors, which revealed that the Bifido FMT group followed a markedly distinct trajectory from the tacrolimus-only controls and Desulfo groups, which instead exhibited similar trajectories (FIG. 18A). The separation was further visualized in the PCoA plots at each time point, where Bifido-treated mice clustered apart from the other groups, establishing Bifido as the only intervention that imposed a sustained divergence in community structure (FIG. 18B). Importantly, distinct changes emerged as early as day 7 and were maintained throughout the study, in line with prior reports of early microbial reprogramming by Bifido.

Species-level heatmaps ordered by time and group revealed coordinated, time-structured responses to Bifido gavage. Bacteroides thetaiotaomicron, a mucosa-associated commensal known to promote Treg induction and anti-inflammatory signaling, remained one of the most abundant taxa through day 28. Bifidobacterium pseudolongum itself peaked immediately post-gavage, then declined to a lower but persistent level, suggesting that repeated administration may be necessary to sustain high-level colonization. Notably, as Bifido abundance waned, several Muribaculaceae lineages, which were enriched in the PBS and Desulfo groups, expanded in a reciprocal manner. This inverse relationship suggests that Bifido may initially suppress Muribaculaceae expansion but that this effect diminishes as its colonization levels decrease. Further, during the Bifido decline, Firmicutes, particularly Lachnospirales, showed a steady increase, indicating broader fermentative capacity increase subsequent to Bifido gavage. Together, these dynamics demonstrated a sustained, system-wide reshaping distinct from the tacrolimus-only state characterized by Muribaculaceae enrichment: Bifido remained abundant for >2 weeks and its ecological impact (e.g., elevated Firmicutes) persisted. The rebound of Muribaculaceae alongside declining Bifido supports repeated Bifido dosing, common in probiotic interventions, to maintain colonization and microbiome reprogramming.

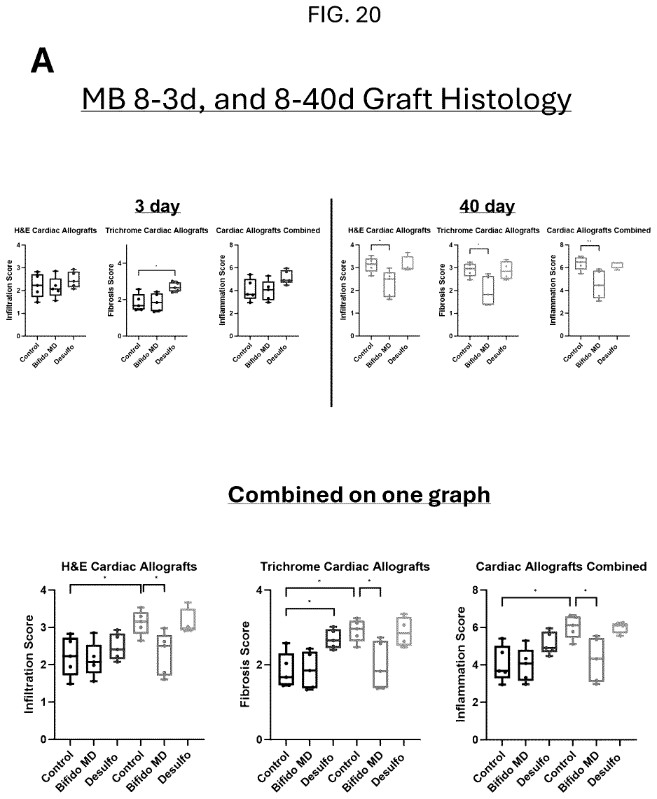

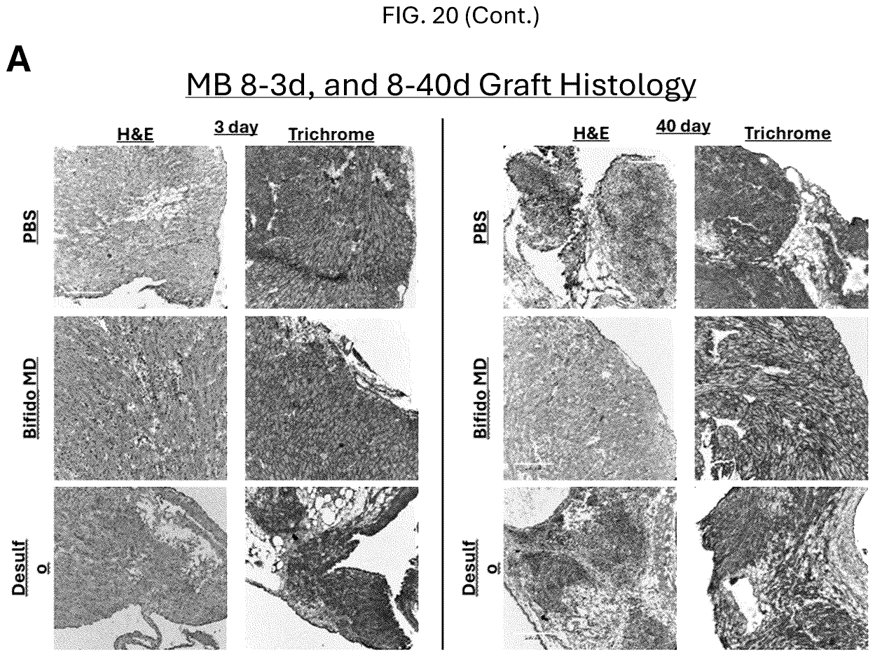

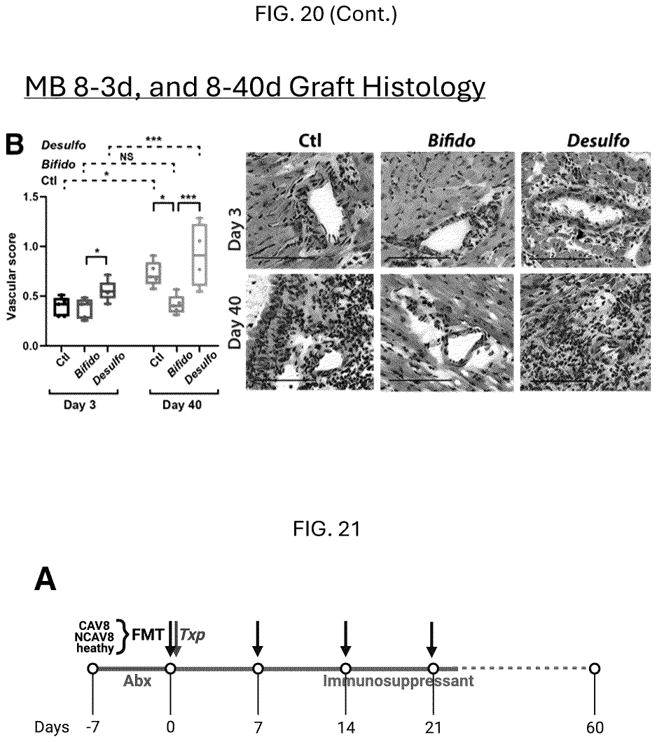

In complementary analyses of graft tissue, control (tacrolimus-only) and Desulfo groups exhibited significantly increased inflammation and fibrosis by day 40, whereas Bifido recipients were comparatively protected (FIG. 20A). Notably, Desulfo showed the highest fibrosis scores as early as day 3 on trichrome-stained cardiac allograft sections, indicating rapid pathologic acceleration. We also evaluated graft vasculature at early (day 3) and late (day 40) time points using the standard cardiac graft-rejection vascular scoring system (FIG. 20B). Desulfo produced rapid and escalating vascular injury, with mild perivascular lymphocytic infiltrates at day 3 that progressed to severe vasculitis by day 40 with intravascular lymphocyte accumulation and vessel wall damage. The tacrolimus-only control followed a similar but milder progressive pattern despite immunosuppression. In sharp contrast, Bifido FMT maintained minimal vascular scores at both time points. These data underscored that persistent, progressive graft vasculitis can occur post-Txp, which immunosuppressant alone cannot fully mitigate, as seen clinically; whereas a protolerogenic microbiome state induced by Bifido preserved vascular integrity and mitigated fibrotic remodeling.

Bifido reproducibly induces dynamic changes in the endogenous gut microbiome and preserves cardiac graft vascular integrity

We repeated the bacterial induction experiment under the same conditions to assess reproducibility. The longitudinal community trajectories recapitulated the original findings, supporting the Bifido significant impact on endogenous gut community, particularly by counteract the Muribaculaceae that is enriched in tacrolimus-only condition (FIG. 18A). Mixed-effects modeling again showed that, comparing to Bifido group, tacrolimus-only controls were significantly enriched for Muribaculaceae, including Muribaculum, CAG-485, Paramuribaculum, and Duncaniella, as well as Erysipelatoclostridium (FIG. 18B). Across longitudinal time point, higher Muribaculaceae abundance correlated inversely with Bifidobacterium levels.

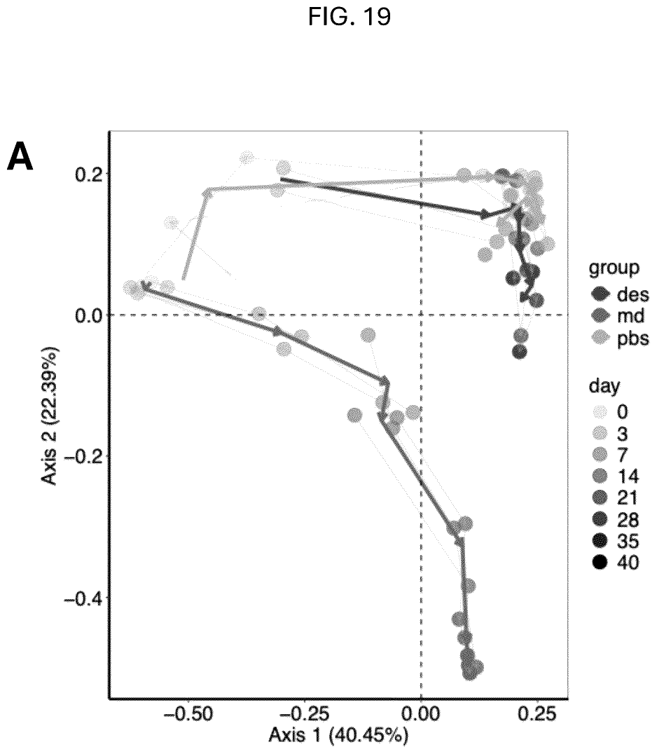

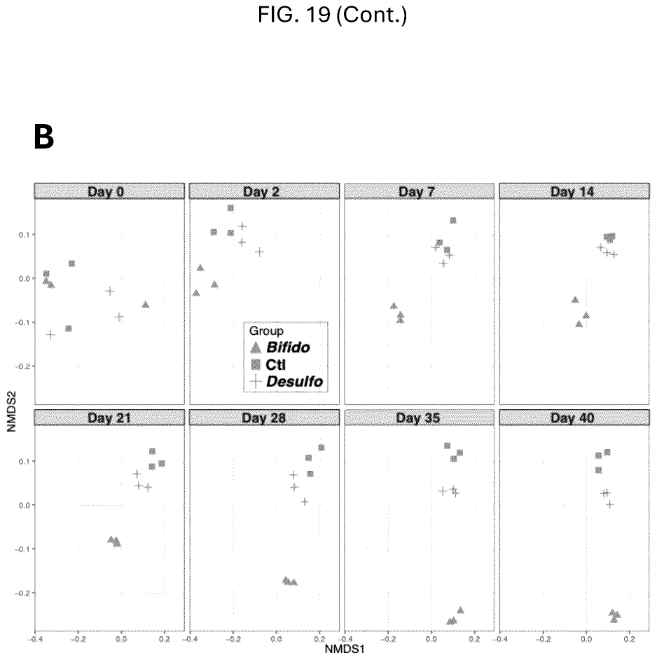

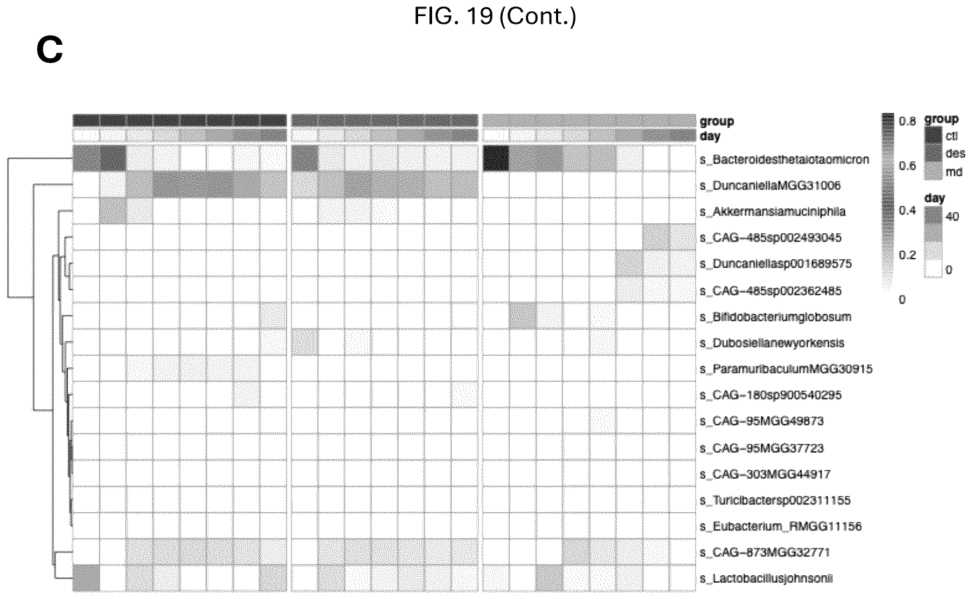

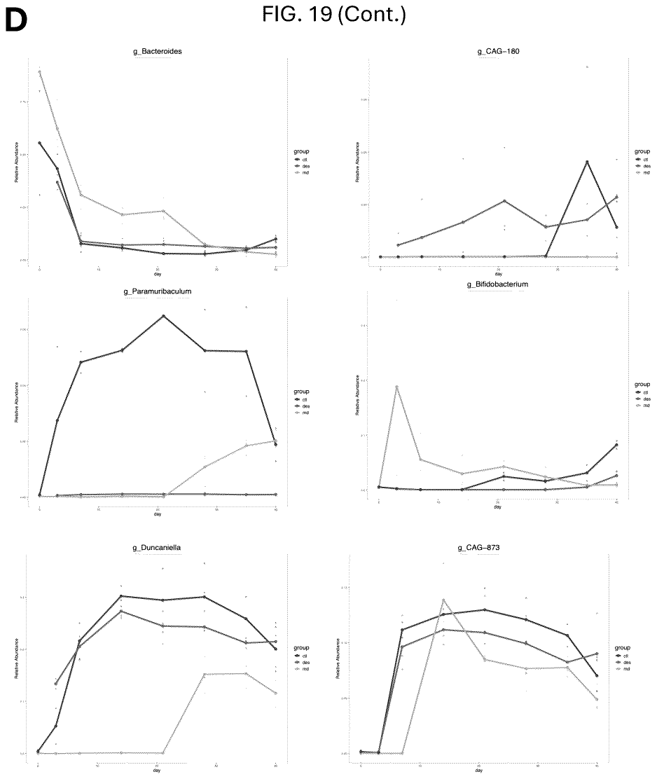

To assess the temporal impact of Bifido on the endogenous gut microbiome after cardiac transplantation, we performed trajectory analysis using PCoA connected by temporal vectors, which revealed that the Bifido FMT group followed a markedly distinct trajectory from the tacrolimus-only controls and Desulfo groups, which instead exhibited similar trajectories (FIG. 19A). The separation was further visualized in the PCoA plots at each time point, where Bifido-treated mice clustered apart from the other groups, establishing Bifido as the only intervention that imposed a sustained divergence in community structure (FIG. 19B). Importantly, distinct changes emerged as early as day 7 and were maintained throughout the study, in line with prior reports of early microbial reprogramming by Bifido.

Species-level heatmaps ordered by time and group revealed coordinated, time-structured responses to Bifido gavage (FIG. 19C). Bacteroides thetaiotaomicron, a mucosa-associated commensal known to promote Treg induction and anti-inflammatory signaling, remained one of the most abundant taxa through day 28. Bifidobacterium pseudolongum itself peaked immediately post-gavage, then declined to a lower but persistent level, suggesting that repeated administration may be necessary to sustain high-level colonization. Notably, as Bifido abundance waned, several Muribaculaceae lineages, which were enriched in the PBS and Desulfo groups, expanded in a reciprocal manner (FIG. 19D). This inverse relationship suggests that Bifido may initially suppress Muribaculaceae expansion but that this effect diminishes as its colonization levels decrease. Further, during the Bifido decline, Firmicutes, particularly Lachnospirales, showed a steady increase, indicating broader fermentative capacity increase subsequent to Bifido gavage. Together, these dynamics demonstrated a sustained, system-wide reshaping distinct from the tacrolimus-only state characterized by Muribaculaceae enrichment: Bifido remained abundant for >2 weeks and its ecological impact (e.g., elevated Firmicutes) persisted. The rebound of Muribaculaceae alongside declining Bifido supports repeated Bifido dosing, common in probiotic interventions, to maintain colonization and microbiome reprogramming.

In complementary analyses of graft tissue, control (tacrolimus-only) and Desulfo groups exhibited significantly increased inflammation and fibrosis by day 40, whereas Bifido recipients were comparatively protected (FIG. 20A). Notably, Desulfo showed the highest fibrosis scores as early as day 3 on trichrome-stained cardiac allograft sections, indicating rapid pathologic acceleration. We also evaluated graft vasculature at early (day 3) and late (day 40) time points using the standard cardiac graft-rejection vascular scoring system (FIG. 20B). Desulfo produced rapid and escalating vascular injury, with mild perivascular lymphocytic infiltrates at day 3 that progressed to severe vasculitis by day 40 with intravascular lymphocyte accumulation and vessel wall damage. The tacrolimus-only control followed a similar but milder progressive pattern despite immunosuppression. In sharp contrast, Bifido FMT maintained minimal vascular scores at both time points. These data underscored that persistent, progressive graft vasculitis can occur post-Txp, which immunosuppressant alone cannot fully mitigate, as seen clinically; whereas a protolerogenic microbiome state induced by Bifido preserved vascular integrity and mitigated fibrotic remodeling.

Human-sourced FMT model reveals CAV-associated expansion of Muribaculaceae and divergent colonization of Bifidobacterium

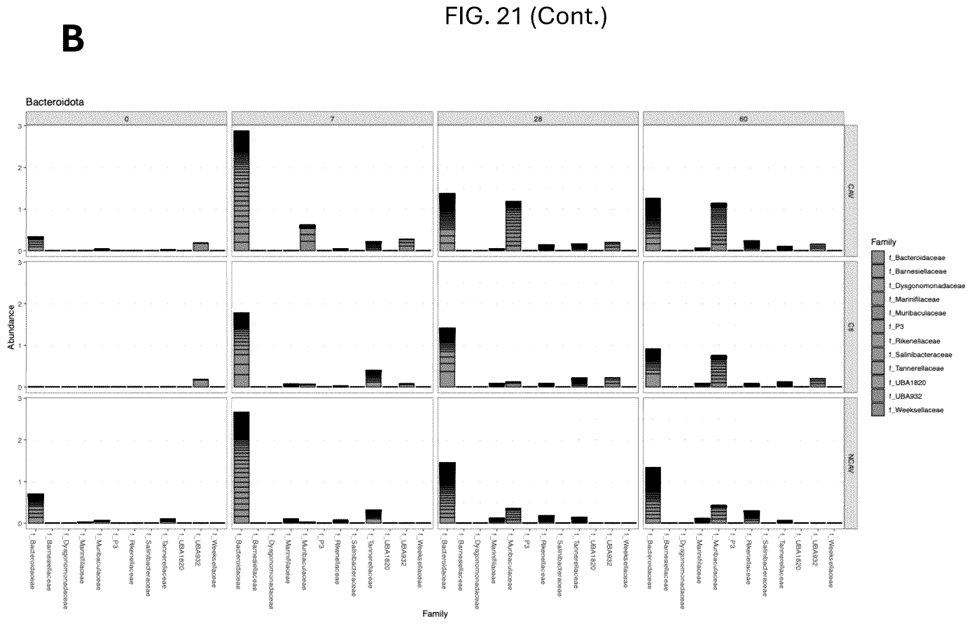

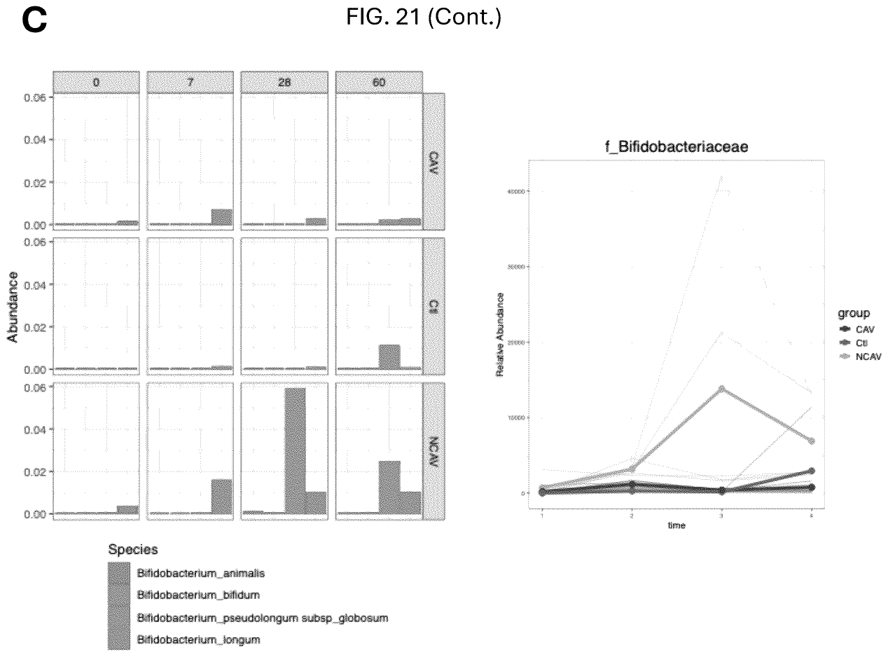

To extend our murine observations to a clinically relevant setting, we performed FMT using stool from cardiac transplant patients with and without cardiac allograft vasculopathy (CAV vs. non-CAV, and healthy subject as a control, FIG. 21A) and collected longitudinal fecal samples on days 0, 7, 28, and 60. Stable engraftment of donor-derived microbiota was achieved across groups, supporting the robustness of the humanized transplant model. Similar to the murine heart transplant model, pro-inflammatory Muribaculaceae emerged after FMT and showed progressive, sustained expansion in the CAV group (FIG. 21B). Taxonomic resolution revealed dominant lineages including Duncaniella and CAG-873, both previously identified as immunogenic taxa enriched in tacrolimus-only conditions and reduced under Bifido FMT. By contrast, Muribaculaceae expansion was delayed and less pronounced in non-CAV and healthy control FMT recipients. Remarkably, because Muribaculaceae are murine endogenously restricted and absent from human donors, their colonization after human FMT indicates reliance on a permissive ecological niche created by FMT induction rather than direct transfer, highlighting a fundamental ecological rule of microbial colonization. In contrast, Bifidobacterium colonization was prominent in non-CAV FMT recipients (FIG. 21C). This comprised a mixture of B. pseudolongum (murine strain) and B. longum (human strain). Although B. longum was not detected in donor stool samples by sequencing, it expanded following FMT, reaching highest abundance in the non-CAV group by day 7 and persisting through day 60, consistent with selective amplification of low-abundance or environmentally introduced taxa under permissive conditions. Notably, B. pseudolongum, the murine-specific strain absent in donor stool, appeared in non-CAV FMT mice by day 28 and was maintained through day 60, suggesting acquisition from the murine endogenous reservoir once permissive conditions induced by FMT is created.

At a taxonomic level comparison, healthy control FMT recipients were enriched for Bacteroides uniformis, Bacteroides vulgatus, Parabacteroides, Blautia, Butyricimonas, Anaerostipes, and Hungatella, consistent with a more balanced community. Non-CAV recipients exhibited increased Ruminococcus, Prevotella, Eubacterium, and UBA-7182 (Lachnospiraceae). In contrast, CAV recipients were enriched for Streptococcus, CAG-873, Clostridium, and UAB-946, as well as Erysipelatoclostridium, a taxonomic also elevated in tacrolimus-only controls in the murine model in addition to Muribaculaceae. Together, these results demonstrated that CAV was associated with Muribaculaceae expansion and immunogenic taxa enrichment (Erysipelatoclostridium), while non-CAV favored Bifidobacterium colonization (B. longum and B. pseudolongum) and SCFA-producing commensals. The findings revealed that ecological context, rather than donor presence alone, dictated colonization outcomes, emphasizing the importance of host- and environment-dependent microbial selection in shaping post-transplant microbiota trajectories.

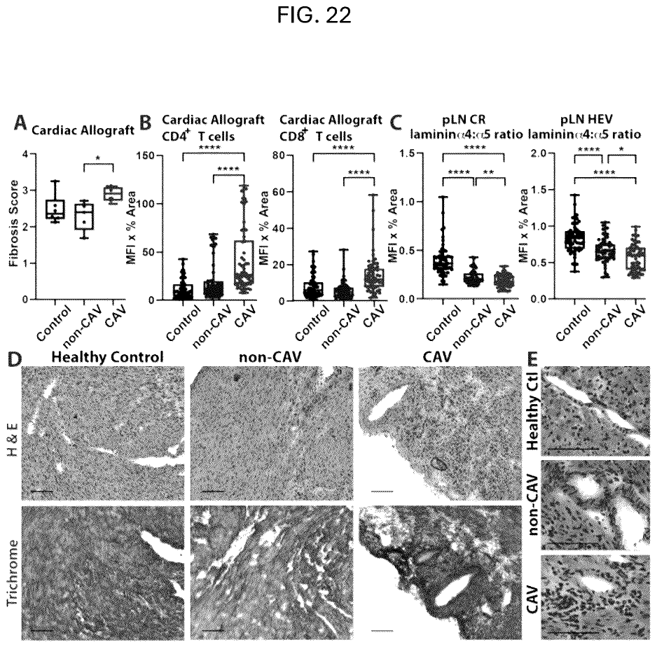

Trichrome staining revealed significantly higher cardiac-allograft fibrosis scores in CAV FMT recipients (FIG. 22A). Immunohistochemistry showed increased CD4⁺ and CD8⁺ T-cell infiltration and a reduced laminin α4:α5 ratio in draining lymph nodes (FIG. 22B), consistent with an effector-biased lymphoid milieu. Assessment of graft blood vessels revealed severe vasculitis in the CAV FMT group, characterized by lymphocytic infiltrates inside or surrounding vessels, a key clinical criterion for grading cardiac graft rejection (FIG. 22C). These data link microbial states with graft pathology in a clinically relevant model.

### Example 3. Multi-Level Gut Modulation to Enhance Immune Tolerance in Cardiac Transplantation

Long-term outcomes after heart transplantation remain suboptimal because chronic rejection and treatment-related complications drive alloimmune injury and compromise long-term allograft survival (Wilhelm, M. J, J Thorac Dis 7, (2015), 549-551; Colvin, M. et al., Am J Transplant 21 Suppl 2, (2021), 356-440; Fishman, J., A. Am J Transplant 17, (2017), 856-879; Gallagher, M. P. et al., J Am Soc Nephrol, (2010), 21:852-858; Schmitz, V. et al., Nephron Exp Nephrol, (2009), 111, e80-91; Bhat et al., Endocr Rev, (2021), 42:171-197). Lifelong immunosuppression, recurrent infections and antimicrobial prophylaxis, and metabolic and organ comorbidities, cumulatively perturb tissue integrity and immune homeostasis. Yet no effective medical strategies exist to monitor, prevent, or treat these predictable effects, representing a critical gap in post-transplant care.

Emerging evidence indicates that these complications arise, at least in part, from unintended disruption of gut homeostasis, characterized by altered gut microbiome, impaired mucosal immunity, and metabolic dysregulation, that ultimately compromise alloimmune regulation and graft tolerance (Faucher, Q. et al., Front Endocrinol (Lausanne), (2022), 13:898878; Gabarre, P. et al., Am J Transplant, (2022), 22:1014-1030; Van Lier et al., Haematologica 106, 2042-2053 (2021); Salvadori et al., World J Transplant, (2024), 14:90194). We recently examined gut and systemic effects of 30-day exposure to five pharmacologically distinct immunosuppressants (calcineurin inhibitor, mTOR inhibitor, glucocorticoid, antimetabolite, and SIPR modulator) in mice (Wu, L. et al., Cell Commun Signal, (2025), 23:506; Wu, L. et al., JCI Insight, (2025), 10; Kensiski, A. et al., Clin Microbiol Rev, (2025), 38:e0017824). Although none of the agents are designed to target the gut, all five induced a conserved gut injury program with convergent microbial dysbiosis characterized by marked expansion of pathobiont Muribaculaceae. Fecal microbiota transfer (FMT) from cardiac allograft vasculopathy (CAV) patients into heart-transplanted mice reproduced this phenotype, consistently creating a niche permissive for Muribaculaceae dominance and metabolic features resembling pharmacologic immunosuppression. This microbial shift co-occurs with gut injury responses, with suppressed mucosal immunity [reduced B cell diversification, immunoglobulin, conventional dendritic cells, T regulatory cells (Tregs)]; epithelial stress reprogramming [antimicrobial peptide (AMP) hyperactivation, circadian resetting, oxidative stress]; metabolic rewiring [elevated toxins such as Trimethylamine N-oxide (TMAO)]; and lymphoid remodeling [mesenteric lymph nodes (mLNs) acquiring pro-inflammatory features and peripheral LN (pLNs) progressively losing tolerogenic architecture]. New show FMT a protolerogenic Bifidobacterium strain significantly reduced Muribaculaceae abundance, restored short chain fatty acids (SCFAs) producers, and expanded Tregs, demonstrating that this dysbiotic state is reversible. Recent studies show that gut epithelial stress programs and mucosal immune responses are monitorable and modifiable (Ma, N. et al., Front Immunol, (2018), 9:5; Lamas et al., Mucosal Immunol, (2018), 11:1024-1038; Wells et al., Am J Physiol Gastrointest Liver Physiol, (2017), 312:G171-G193; Shi et al., Mil Med Res, (2017), 4:14. Chronic immunosuppressant exposure progressively drives a gut microbiota-epithelial-immune cascade that can be monitored and modulated at multiple levels to limit complications and alloimmune injury, thereby improving chronic allograft outcomes. The goal of this example is to identify interventions that are both effective and well tolerated in models explicitly designed to operate alongside immunosuppressant therapy. To maximize safety for immunocompromised recipients, we will prioritize non-live interventions (“postbiotics”) over live probiotics (Lamas et al., Mucosal Immunol, (2018), 11:1024-1038), focusing on metabolic compounds with protolerogenic features and existing safety and tolerability data, to target (Part 1) microbial dysbiosis, (Part 2) epithelial stress/metabolic rewiring, and (Part 3) lymphatic-immune niche:

Part 1: Target immunosuppression-induced gut dysbiosis to restore intestinal homeostasis. 1A) Preclinical testing. In a fully allogeneic, tacrolimus-maintained cardiac transplant mouse model, we will test co-administrable postbiotics (adenosine, spermidine, butyrate, indole-AhR ligands, NAD+ boosting) to recondition the intestinal niche, quantify efficiency with a predefined dysbiosis index, and establish sufficiency and necessity using targeted antagonists (e.g., A2aR, CD73 blockade). 1B) Translational Validation. In cardiac transplanted mice receiving FMT from CAV patients, we will administer the compositions for efficacy and durability. We expect the compositions will deliver therapeutic leads that normalize gut dysbiosis, restore mucosal immune regulation, and attenuate graft inflammation/fibrosis.

Part 2: Target intestinal epithelial injury and metabolic dysregulation. We will 2A) develop a surveillance toolkit to track (i) epithelial injury, via a stool-based stress signature (AMP, redox/oxidative, circadian, barrier), and (ii) metabolic dysregulation, via a serum panel (tissue injury, toxicity-associated, inflammation, fibrogenesis); and 2B) reset epithelial homeostasis in tacrolimus-treated cardiac allografts using strategies of cytokine repair (IL-22), epithelial receptor pathways (AhR/FXR), antioxidant defenses (NRF2), and short chain fatty acid SCFAs-based diet. We expect to deliver biomarker-guided regimens that neutralize immunosuppressant induced epithelial injury, stabilize the host-microbe interface, and reduce gut-driven systemic toxicity.

Part 3: Target human Treg induction and stability in a gut-lymphatic endothelial niche. Because gut-derived metabolites traverse lymphatic endothelium en route to gut-draining mLNs, we will test whether tuning selected metabolic pathways during T-cell transendothelial migration (TEM) across lymphatic endothelial cells (LECs) biases CD4⁺ T cells toward durable regulatory programs. Using a primary human LEC TEM transwell model, we will test whether metabolic targets 3A) are sufficient/necessary for induction of FOXP3+Tregs from naïve CD4⁺CD25low/-CD127 hi T cells (adenosine/purine, AhR ligands, spermidine±matched inhibitors), and 3B) confer stability by preserving FOXP3+ retention/suppression in CD4⁺CD25⁺CD127low Tregs under destabilizing cues (e.g., IL-6), with validation in Tregs from transplant patient PBMCs. We expect to define targetable metabolic pathways that reliably induce and stabilize Tregs under inflammatory and immunosuppressive stress.

We aim to deliver co-administrable interventions that mitigate gut, epithelial, and immune injury, shifting post-transplant care from reactive management of gut toxicities to proactive preservation of immune tolerance.

### Example 4: Dynamic Metabolic Rewiring Determines Immune Outcomes of Chronic Cardiac Transplant Outcomes

Cardiac transplant recipients ubiquitously develop cardiac allograft vasculopathy, and the mortality rate beyond the first year has not changed in two decades. Using a preclinical murine model and human-sourced fecal microbiota samples, this example will define mechanisms by which microbiome-driven metabolic alterations impact alloimmunity and determine graft outcomes. Findings will identify novel microbial and metabolic targets translatable to human, to induce sustained immune quiescence and improving graft acceptance.

Cardiac transplant recipients ubiquitously develop cardiac allograft vasculopathy (CAV), interstitial fibrosis and chronic inflammation, with post-transplant mortality rate unchanged for the past two decades. Immune-mediated damage is the primary cause of long-term graft failure. The pathophysiology of chronic graft rejection is still poorly understood; and tools to predict, prevent, and treat graft chronic inflammation and fibrosis are largely lacking. Emerging evidence indicates that the microbiome and microbiome-regulated metabolic compounds critically impact alloimmunity, consequently promoting or inhibiting graft survival. We developed a clinically relevant murine model of chronic cardiac rejection, characterized by full MHC mismatch, administration of clinically relevant chronic immunosuppression, and development of chronic cardiac lesions. With this model we showed pro- and anti-inflammatory bacteria and whole gut microbiota can be defined by their effects on graft outcome: anti-inflammatory Bifidobacterium (Bifido) improved long-term graft outcome, while pro-inflammatory Desulfovibrio (Desulfo) impaired graft survival, each influencing lymph node (LN) structure, lymphocyte trafficking and regulatory T cell (Treg) differentiation. Bifido and Desulfo distinctively reprogrammed metabolic activities in gut lumen and circulation, affecting amino acid, carbohydrate and nucleotide metabolism, and trimethylamine biosynthesis. Remarkably, FMT from a human-sourced cardiac transplant recipient with CAV, using this murine model, resulted in pro-inflammatory responses akin to those seen with Desulfo FMT. We propose that changes in gut metabolic activities, due to altered gut microbiome, shift the immune balance of Tregs vs. effector T cells, thereby inhibiting or promoting transplant rejection. Specifically, gut metabolic activities regulate the LN microenvironment where the Treg/Teff ratio is determined.

The goal of this example is to determine microbiome-mediated changes that serve as key determinants of alloimmune modulation and CAV development. We will use our clinically relevant murine model to derive insights pertinent to metabolic and immunological parameters of human transplantation.

In Part 1, we will define microbiome-induced metabolic reprogramming in cardiac transplantation.

In Part 2, we will determine metabolically driven alloantigen-specific responses in lymphoid tissues and grafts. In Part 3, we will characterize the metabolic and alloimmune responses induced by fecal microbiota transfer sourced from human cardiac transplant recipients under immune regulatory (non-CAV) or activated (CAV) conditions. The results of this example will define translatable microbial- and metabolite-informed targets for monitoring, diagnostics and therapeutics in transplantation, and discern the complex dialogue between microbiome and alloimmunity.

This example 1) introduces a novel, microbiome informed explanation for chronic graft inflammation and fibrosis, addressing previously unexplained variations in transplant outcomes, with findings applicable across various organ transplants; 2) includes parallel characterization of microbial induction from both murine pro-tolerogenic vs. pro-inflammatory bacteria and human donors with immune-activating vs. immuneregulatory profiles, to identify novel, clinically translatable targets that determine alloreactivity and graft outcomes; and 3) demonstrates microbial induction influencing LN architecture, function, and T cell responses, moving beyond associative relationships to causal mechanisms of how local gut interactions can be translated to regional and systemic alloimmune alterations. Technically, it utilizes 1) hypothesis-driven investigation of metabolic effects using an agonist/antagonist experimental design to establish causality; 2) continuous 60-day monitoring of chronic cardiac rejection in murine models to define features signaling the onset and progression of CAV within experimentally trackable timeframes; and 3) scenarios of pre-transplant prophylactic prevention, peri-operative intervention, and post-transplant therapeutics, to evaluate intervention efficacy.

We propose that alterations in the gut microbiome drive metabolic changes that shift the balance between Tregs vs. T effectors (Teffs), mediating allograft quiescence versus injury as a key determinant in CAV development. The goal of this example is to determine microbiome mediated changes that serve as key determinants of alloimmune modulation and the development of CAV. We will employ our murine model of chronic allograft rejection, induced by pro- or anti-inflammatory bacteria to investigate (Part 1) metabolic reprogramming and (Part 2) alloantigen-specific responses that determine allograft quiescence vs. injury. In parallel, (Part 3) we will define metabolic and alloimmune responses induced by FMT sourced from cardiac transplant patients with CAV (immune activated) and without CAV (immune regulatory).

### Results

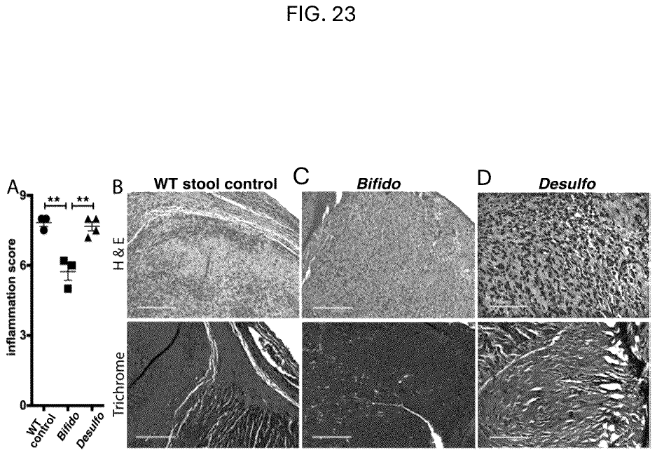

Single bacterial strains alone influence graft inflammation and survival similar to the whole microbiota (Gavzy, S. J. et al., Transplantation, (2024), doi:10.1097/TP.0000000000004939; Ma, B. et al. Sci Rep, (2023), 13:1023; Bromberg et al., JCI Insight, (2018), 3, doi:10.1172/jci.insight.121045; Mongodin, E. F. et al., Genome Announc, (2017), 5, doi:10.1128/genomeA.01089-17). Using our cardiac transplant murine model, FMT of whole stool from colitic (inflamed) or pregnant (immune suppressed) donors resulted in impaired vs improved allograft outcomes, respectively. Bifido was revealed to significantly associated with pregnant FMT and Desulfo with colitic FMT. Further, graft recipients receiving Bifido or Desulfo alone, which were isolated from whole stool, recapitulated the whole stool FMT effects from pregnant or colitic mice, respectively (FIG. 23), identifying these bacteria as key determinants of phenotypic changes.

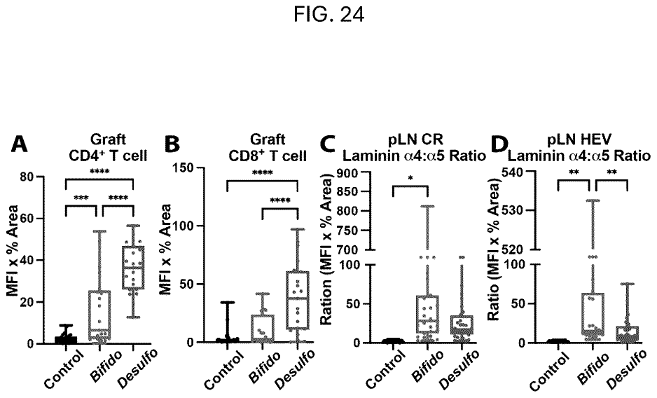

Bacterial determinants alter regional and systemic immune responses (Gavzy, S. J. et al., Transplantation, (2024), doi:10.1097/TP.0000000000004939; Ma, B. et al. Sci Rep, (2023), 13:1023; Bromberg et al., JCI Insight, (2018), 3, doi:10.1172/jci.insight.121045). A preferential stimulation of DC and M inflammatory cytokine and chemokine responses by Desulfo compared to Bifido were observed, resulting in LN structural changes that influenced allogeneic immunity. In C57BL/6 recipients of BALB/c cardiac transplants (Txp) and Bifido or Desulfo FMT, grafts from Desulfo-treated mice showed increased CD4+ helper and CD8+ cytotoxic T cells within 7 days (FIG. 24A-B), while Bifido resulted in higher LN laminin α4:α5 ratios in CR and HEV (FIG. 24C-D), indictive of inflammatory vs. suppressive environments, respectively (Warren et al., J Clin Invest, (2014), 124:2204-2218; Simon et al., Trends Immunol, (2017), 38, 858-871; Simon, T. et al. Transplantation, (2019), 103:2075-2089).

Bacterial determinants alter alloantigen specific CD4 T cell responses (Gavzy, S. J. et al., Transplantation, (2024), doi:10.1097/TP.0000000000004939; Bromberg et al., JCI Insight, (2018), 3, doi:10.1172/jci.insight. 121045). To determine alloantigen specific responses, mice were treated as above along with adoptive transfer of T cell receptor transgenic TEa CD4+ T cells (2×10⁵ cells i.v.) specific for donor I-E^{d} alloantigen. After 7 days, Bifido significantly increased TEa Foxp3+ Tregs in LNs and decreased activated CD44hiCD69+ T effector cells in LNs and spleens. Bifido treated mice had increased naive CD4+CD44-CD62L+CD69-TEa T cells, and a lower percentage of effector memory CD4+CD44+CD62L+CD69-TEa T. In contrast, Desulfo FMT resulted in opposite effects, with increased T cell activation and reduced Treg induction. These results delineate the profound influence of specific bacteria determinants on immunity and LN structure, affecting adaptive regional and systemic immunity (Fonseca, D. M. et al., Cell, 163:354-366).

CCR2 antagonism blocks bacterial pro-inflammatory effects on LN (Gavzy, S. J. et al., Transplantation, (2024), doi:10.1097/TP.0000000000004939). Since Bifido and Desulfo FMT had differential effects on DC and MΦ responses, and CCR2-mediated myeloid cell homing from gut to LN may contribute to transmission of microbiota-specific effects on the immune system, we investigated CCR2 inhibition. C57BL/6 mice were treated as above along with CCR2 antagonist (RS504393, 2 mg/kg/d s.c.) for 7 days. RS504393 abrogated the pro-inflammatory effect of Desulfo FMT on LN architecture, so the laminin α4:α5 ratio increased rather than decreased compared to control (p<. 0001). These findings implicate CCR2 mediated homing of inflammatory myeloid cells from gut to LN causing architectural changes after Desulfo FMT, and tolerogenic myeloid cell homing after Bifido FMT, as a mechanism of microbiota induced immune modulation.

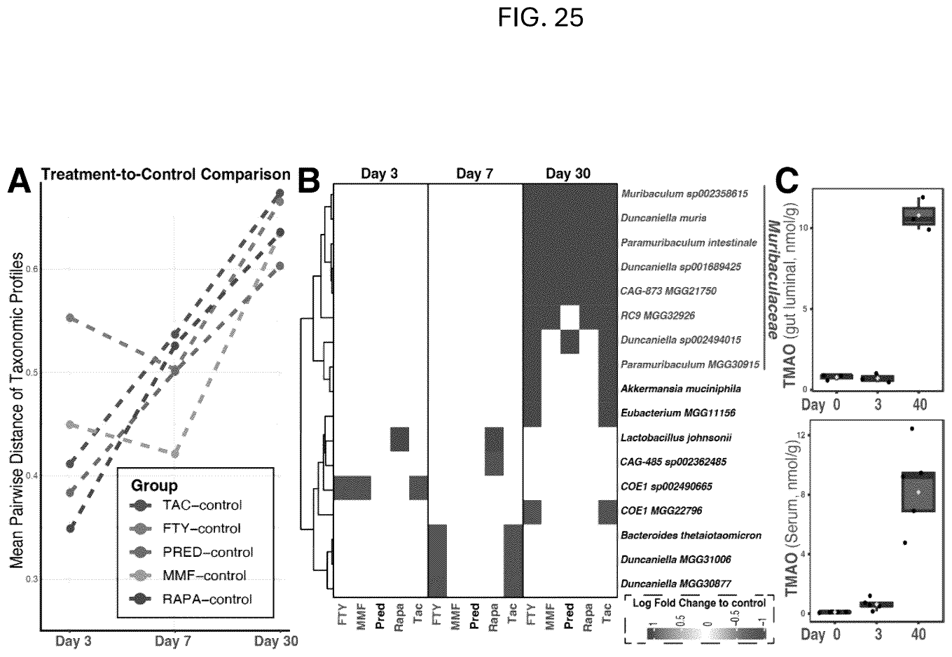

Chronic immunosuppressive therapy induces gut dysbiosis and metabolic dysregulation (Wu, L. et al., bioRxiv, (2025), 2025.2001.2002.631100; Wu, L. et al. JCI Insight, (2025), 10, doi:10.1172/jci.insight. 186505; Kensiski, A. et al., Clin Microbiol Rev, (2025), e0017824, doi:10.1128/cmr.00178-24). To define the baseline microbiome shifts induced by immunosuppressants, we profiled the gut microbiome at 3-, 7-, and 30-day following treatment with five major classes of immunosuppressants: calcineurin inhibitors, mTOR inhibitors, glucocorticoids, anti-metabolites, and SIPR modulators. Although mechanistically distinct and not designed to target the gut, all five immunosuppressants induced progressive microbiome changes over time (FIG. 25A), ultimately converging by day 30 on a shared taxonomic signature marked by significant expansion of Muribaculaceae (FIG. 25B). Muribaculaceae are a highly metabolically versatile pathobiont family, conferring a competitive advantage in disrupted gut environments (Ormerod, K. L. et al. Microbiome, (2016); Lagkouvardos, I. et al., Microbiome, (2019), 7:28; Smith et al., mSphere, (2021), 6:e0085121). They have been causally linked to insulin-dependent diabetes and impaired glucose metabolism (Yang, X. et al., Microbiome, (2023), 11:62; Chang et al., J Inflamm Res, (2021), 14:6237-6250). Metabolomic analyses of intraluminal stool after 30-day tacrolimus treatment revealed elevated levels of free amino acids (e.g., arginine, tryptophan, histidine) and reduced level of polyamines (e.g., spermidine, N¹-acetylspermidine, N¹, N¹²-diacetylspermine), suggesting impaired amino acid utilization. Significant disruptions were also observed in nucleotide metabolism, short-chain fatty acids (SCFAs), and trimethylamine (TMA)/trimethylamine N-oxide (TMAO) pathway. Serum metabolomic analyses corroborate these findings, revealing elevated circulating TMAO levels (FIG. 25C). These findings indicate that chronic immunosuppression disrupts microbial-derived metabolism in both gut and circulation, alterations that may underlie the vascular inflammation and off-target complications commonly observed in transplant patients.

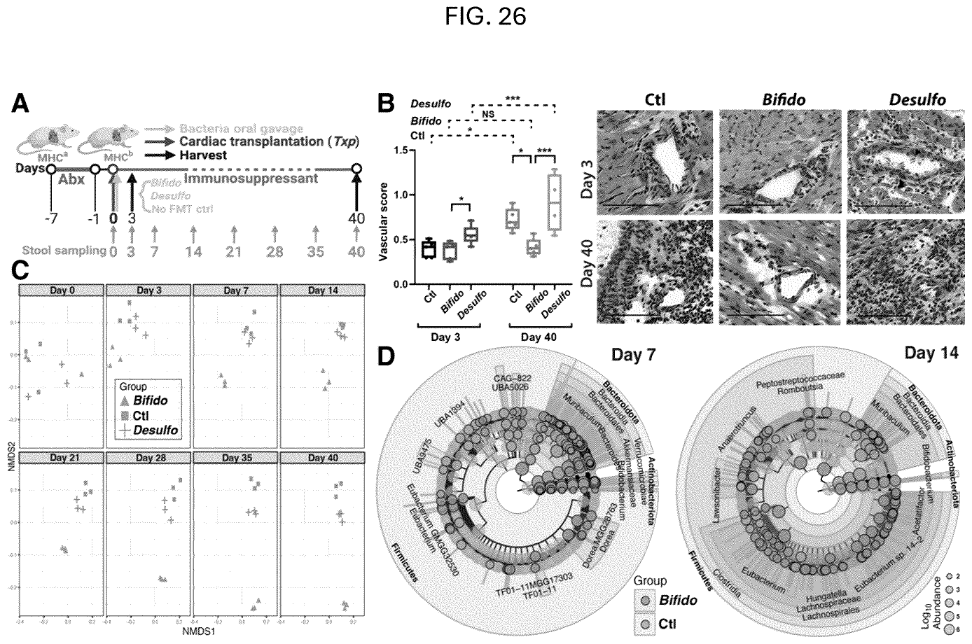

Bacterial determinants influence graft vascular inflammation. We next investigated the impact of microbial induction on graft vasculature at days 3 and 40 (FIG. 26A). Using the vascular scoring system based on standard cardiac graft rejection grading⁴, we observed striking differences in graft vasculopathy between pro-inflammatory and pro-tolerogenic bacteria FMT (FIG. 26B). Desulfo FMT led to rapid and escalating vascular inflammation evident by day 3, with mild perivascular lymphocyte infiltrates, progressing to severe vasculitis by day 40 with intravascular lymphocyte infiltration and significant vessel wall damage. The no-FMT control group showed a similar, albeit milder, progressive pattern despite the use of immunosuppression. In contrast, Bifido FMT maintained minimal vascular scores throughout. This showed persistent and progressive graft vascular inflammation following transplantation, which immunosuppressants alone fail to control, reflecting their limited ability in preventing chronic vasculopathy as seen in clinical settings. Overall, these results demonstrate that detrimental microbial profiles can rapidly initiate and perpetuate pathological processes leading to graft vasculopathy, whereas beneficial microbes preserve graft vascular integrity.

Rapid, sustained impact of microbial induction on the endogenous gut microbiome. A longitudinal analysis over 40 days post-Txp with chronic immunosuppression revealed a progressive shift in the endogenous gut microbiome. Bifido FMT induced distinct microbiome changes by day 7 that persisted throughout the study (FIG. 26C). “Early responders” included increased B. thetaiotaomicron, which promotes anti-inflammatory Tregs and Th2 while suppressing pro-inflammatory Th1 and Th17, and Clostridia taxa (FIG. 26D) (Li, K. et al., Int Immunopharmacol, (2021), 90:107183). Concurrently, we observed a depletion of Muribaculaceae, Erysipelotrichaceae, immunogenic bacteria linked to inflammatory conditions including colitis, metabolic syndrome, and accelerated graft-versus-host disease (Bowerman, K. L. et al., Gut microbes, (2020), 11:754-770; Forster, S. C. et al., Nat Microbiol, (2022), 7:590-599; Craven, M. et al., PLOS One, (2012), 7:e41594; Schaubeck, M. et al., Gut, (2016), 65:225-237; Palm, N. W. et al., Cell, (2014), 158:1000-1010). “Late responders” included sustained depletion of Muribaculaceae and a significant expansion of Clostridia, particularly Lachnospiraceae and Eubacterium. These are potent anaerobic fermentators of carbohydrates and amino acids, producing compounds like SCFAs that attenuate inflammation, strengthen intestinal barrier123, and induce Treg-dependent donorspecific tolerance (Atarashi, K. et al., Science, (2011), 331:337-341; Wu, H. et al., J Am Soc Nephrol, (2020), 31:1445-1461; Sokol, H. et al., Proc Natl Acad Sci USA, (2008), 105:16731-16736; Kiely et al., Gut microbes, (2018), 9:477-485; Atarashi, K. et al., Nature, (2013), 500:232-236). Notably, Muribaculaceae has significant expansion following long-term immunosuppressant treatment (FIG. 25B) and was consistently depleted after Bifido FMT (FIG. 26D and (Ma, B. et al. Sci Rep, (2023), 13:1023)), suggesting that Bifido FMT may mitigate immunosuppressantinduced pathobiont expansion and restore an anti inflammatory environment conductive to immune regulation.

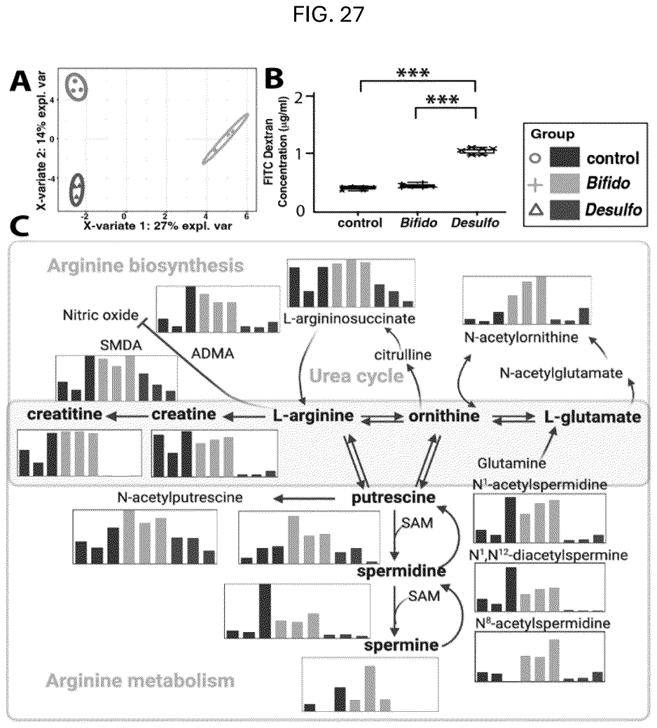

Bacterial determinants induce metabolic reprogramming. We next examined gut luminal metabolism on day post-Bifido or Desulfo FMT with immunosuppressants and revealed distinct metabolic profiles (FIG. 27A). Bifido FMT profoundly modulated pathways of glycolysis, nucleotide metabolism (purine, pyrimidine), and amino acid metabolism (e.g., tryptophan, arginine, and histidine). Interestingly, these amino acid substrates accumulate in the gut lumen following long-term immunosuppressant treatment (Wu, L. et al., bioRxiv, (2025), 2025.2001.2002.631100; Wu, L. et al. JCI Insight, (2025), 10:186505; Kensiski, A. et al., Clin Microbiol Rev, (2025), e0017824). Bifido FMT significantly increased amino acids metabolic products, including histamine (from histidine), kynurenine and kynurenic acid (from tryptophan), and polyamines such as putrescine, spermidine, spermine (from arginine) (FIG. 27C), suggesting that Bifido restores critical microbial metabolic functions disrupted during chronic immunosuppression posttransplant. Polyamines, particularly spermidine and spermine, have been shown to promote Treg development and tip the balance between Th17 and Foxp3+ Treg cells125,126. Metagenomic analyses revealed that both Bifido and B. thetaiotaomicron, a key species responsive to Bifido FMT, harbor pathways that convert ornithine to spermine and degrade L-histidine into glutamate. This finding aligns with a recent human study demonstrating that arginine and histidine metabolism are immunomodulatory pathways mediated by B. longum, a human counterpart to murine B. pseudolongum (Bifido) (Strazar, M. et al., Nat Commun, (2021), 12:4845).

In contrast, Desulfo FMT increased intestinal permeability (FIG. 27B), elevated TMA and oxidative stress, and increased primary bile acid (BAs) without a corresponding increase in secondary BAs. Impaired BA conversion is linked to inflammation, neoplasia, acute cardiac rejection, and hepatic damage (Lin et al., Sci Rep, (2017), 7:15422; Duboc, H. et al., Gut, (2013), 62, 531-539; Ridlon et al., Gut microbes, (2016), 7:22-39; Collins et al., Nat Rev Microbiol, (2023), 21:236-247; Mohanty, I. et al., Nat Rev Gastroenterol Hepatol, (2024), doi:10.1038/s41575-024-00914-3; Keskitalo, A. et al., PLOS One, (2018), 13, e0198262). Moreover, Desulfo not only generate proinflammatory hydrogen sulfide but utlize an anaerobic choline metabolism pathway via choline TMA-lyase, generating TMA and contributing to elevated TMAO and atherosclerosis risk (Koeth, R. A. et al., Nat Med, (2013), 19:576-585; Heidelberg, J. F. et al., Nat Biotechnol, (2004), 22:554-559; Martinez-del Campo, A. et al., MBio, (2015), 6:doi:10.1128/mBio.00042-15; Bennett, B. J. et al., Cell Metab, (2013), 17:49-60). These results support the concept that microbiome-mediated immune modulation corresponds with specific metabolic shifts, and Bifido FMT can mitigate immunosuppressant-induced dysbiosis and metabolic disruption.

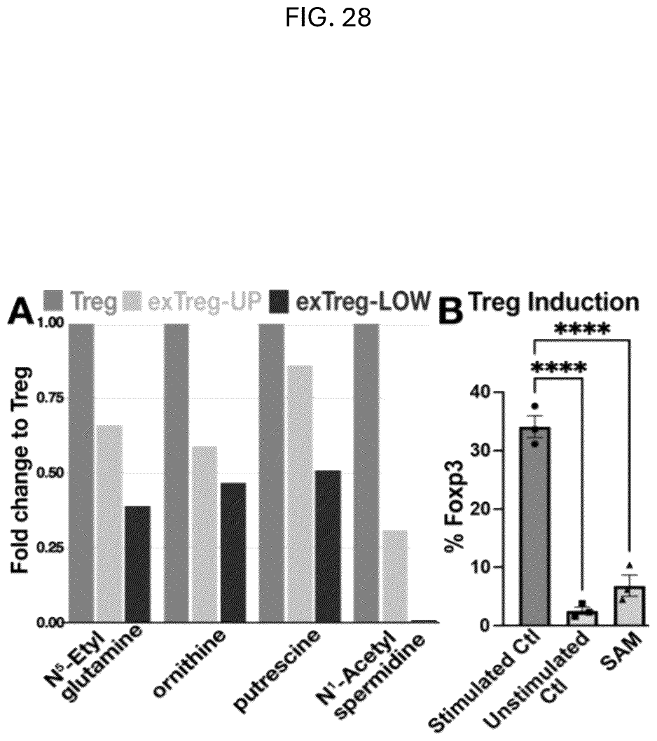

Polyamines drive stability and function of Tregs (Saxena, V. et al., Cell Rep, (2022), 39:110727). Treg interactions with lymphatic endothelial cells (LEC) determine Treg stability and suppressive function through lymphotoxin (LT) αβ and ectonucleotidases CD39 and CD73 (all expressed on Tregs), and LTβR, IL-6, and adenosine 2AR (all expressed by LEC). Adenosine generated by Tregs is important for Treg stability as it alters LEC secretion of IL-6. Metabolomic profiling of stable Tregs versus unstable exTregs induced by LEC showed that Tregs had higher levels of polyamines (putrescine, ornithine, spermidine) and glutamine; and all were decreased in exTregs (FIG. 28A) (Carriche, G. M. et al., J Allergy Clin Immunol, (2021), 147:335-348, e311; Wu, R. et al., Sci Adv, (2020), 6:doi:10.1126/sciadv.abc4275; Puleston, D. J. et al., Cell, (2021), 184:4186-4202, e4120). S-adenosylmethionine (SAM) is a cofactor for conversion of putrescine to spermidine and spermine, and SAM decreases putrescine levels (Stjernborg et al., Eur J Biochem, 214, 671-676, (1993)). Indeed, SAM inhibited Treg induction (FIG. 28B). Together the data suggest polyamine homeostasis contributes to a metabolic environment that supports Treg induction and stability.

Gut-circulation metabolic correlation is based on the premise that the intestinal vascular system distributes luminal metabolic products systemically, while gut-conditioned immune cells are influenced by factors such as medications, gut microbiome, and immune status (Ma, B. et al., BMC Microbiol, (2023), 23:394; Gentile et al., PLOS Pathog, (2018), 14:e1007045; Matheson et al., J Surg Res, (2000), 93, 182-196).

Prior studies have shown that serum metabolites, such as glycolysis metabolites and amino acids derivatives, can predict gut microbiota features; likewise, gut microbiome-derived metabolites such as TMA and TMAO correlate with circulating immune cells and inflammatory markers relating to cardiovascular health and vascular endothelial functions (Wilmanski, T. et al. Nat Biotechnol, (2019), 37:1217-1228; Vojinovic, D. et al., Nat Commun, (2019), 10:5813; Wang, Z. et al., Microbiome, (2023), 11:119; Meng, Q. et al. Gut microbes, (2021), 13:1-27; Battson, M. L. et al. Am J Physiol Endocrinol Metab, (2018), 314:E468-E477). We analyzed paired intraluminal stool (local) and serum (systemic) metabolome after abx and/or tacrolimus and revealed a strong correlation (Ma, B. et al., BMC Microbiol, (2023), 23:394). The most correlated metabolites are involved in glycolysis, carbohydrate and amino acid metabolism, nitric oxide regulation, and Bas metabolism. These findings suggest a mechanistic link between gut metabolism and systemic effect, contributing to alloimmune responses and vascular outcomes.

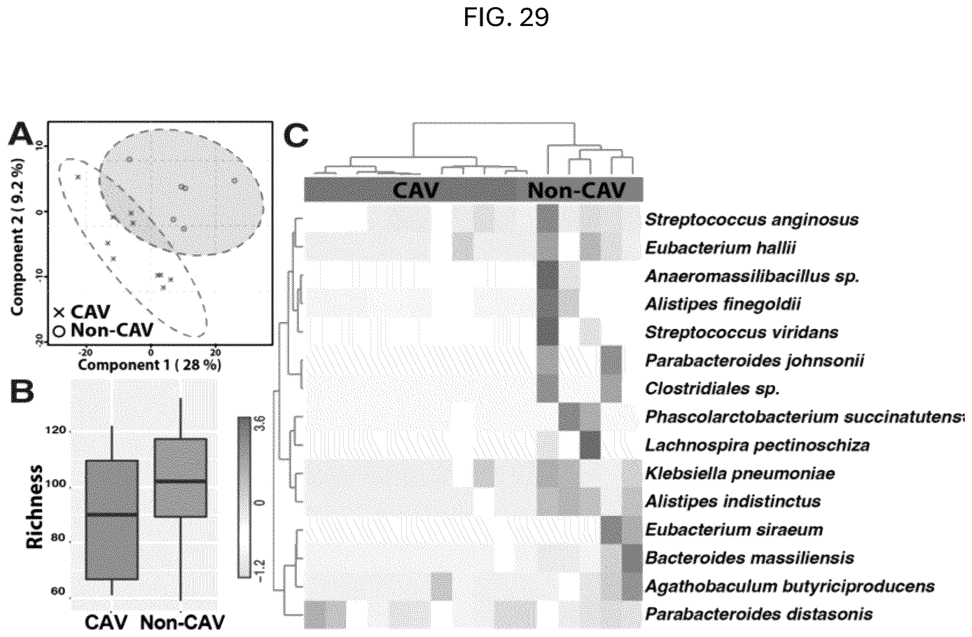

Gut dysbiosis in human CAV patients was observed in a pilot cohort of cardiac transplant recipients (10 with CAV, 6 without CAV). Despite the limited sample size and lack of confounder modeling, human CAV patients exhibited a distinct microbiota profile (FIG. 29A). Both groups showed markedly reduced microbial diversity, about 10%-50% of that in the general population in the Human Microbiome Project149, and CAV patients had even lower diversity (FIG. 29B), likely reflecting their inflammatory state. CAV were enriched in Parabacteroides distasonis (FIG. 29C), an aerotolerant microbe linked to autoimmune responses (Zhou, C. et al. J Autoimmun, (2020), 107; 102360; Cekanaviciute, E. et al., Proc Natl Acad Sci USA, (2017), 114:10713-10718). In contrast, non-CAV had significantly higher levels of Bacteroides and Clotridium cluster XIVa and IV (Lachnospiraceae, Eubacteriaceae), known for anaerobic fermentation of carbohydrate and proteins, or potent SCFA producers.

Notably, these clostridial groups align with the “late responders” to Bifido FMT (FIG. 26D). These data suggest CAV is associated with reduced microbial diversity, less strict anaerobes, and impaired fermentative metabolism.

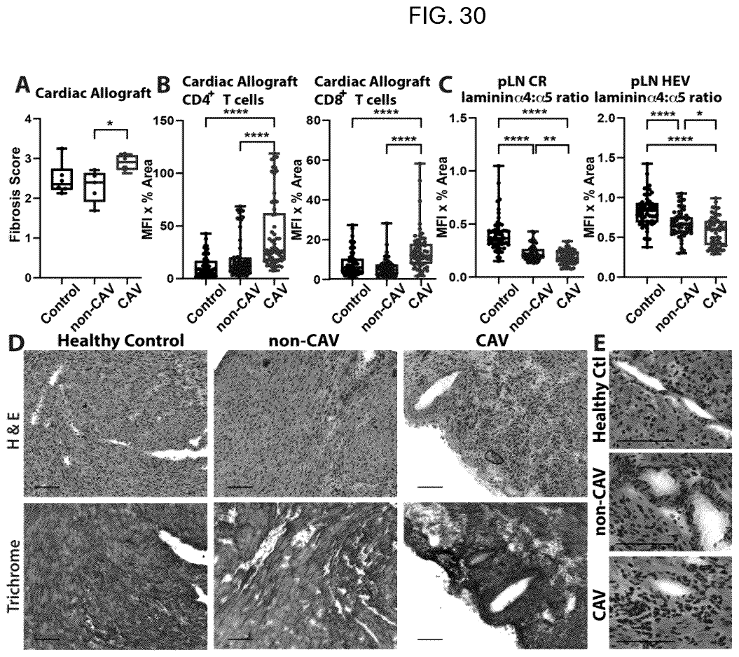

FMT from CAV patients induces pro-inflammatory allograft changes in the murine model. To translate mechanistic findings to human, we performed FMT from cardiac transplant subjects with and without CAV and a healthy control to our murine model. FMT from the CAV patient resulted in significantly increased fibrosis (FIG. 30A, D), CD4+ and CD8+ T cell infiltration in allografts (FIG. 30B), indicating a strong proinflammatory influence. Peripheral LN showed reduced laminin α4:α5 ratios (FIG. 30C), suggesting an immune-stimulated environment and remodeling. Assessment of graft blood vessels revealed severe vasculitis in CAV FMT group, characterized by lymphocytic infiltrates inside or surrounding vessels, a key clinical criterion for grading cardiac graft rejection (FIG. 30E) (Stewart, S. et al., J Heart Lung Transplant, (2005), 24, 1710-1720). These findings support the clinical relevance of our murine model, highlighting the determinant role of gut microbiome in key immunological events influencing graft fibrosis and vascular inflammation.

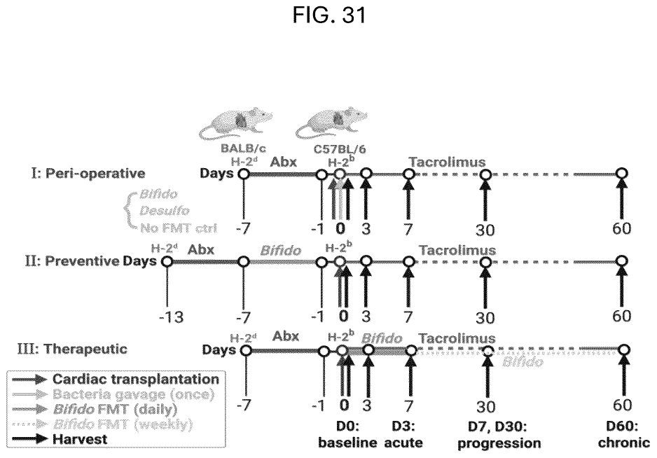

Experimental Methods. FIG. 31 will follow our established protocols (Gavzy, S. J. et al., Transplantation, (2024), doi:10.1097/TP.0000000000004939; Ma, B. et al. Sci Rep, (2023), 13:1023; Bromberg et al., JCI Insight, (2018), 3, doi:10.1172/jci.insight.121045; Bokoliya et al., Front Cell Infect Microbiol, (2021), 11:711055; Barcena, C. et al., Nat Med, (2019), 25:1234-1242). Mice will be cohoused and handled together for at least 10 days to normalize environment exposure and ensure a shared baseline. On DO, they will be randomly assigned to FMT groups and housed separately thereafter to prevent cross-exposure. Three longitudinal designs will be used: peri-operative, preventive, and therapeutic evaluation. (I) Peri-operative design administers bacterial FMT on the day of Txp (DO), following our establish protocols and as in FIG. 26 (Gavzy, S. J. et al., Transplantation, (2024), doi:10.1097/TP.0000000000004939; Ma, B. et al. Sci Rep, (2023), 13:1023; Bromberg et al., JCI Insight, (2018), 3, doi:10.1172/jci.insight.121045). (II) The preventive design evaluates whether pre-Txp Bifido FMT can precondition the endogenous gut microbiome, thereby establishing a protective condition before Txp (Jalanka, J. et al., Aliment Pharmacol Ther, (2018), 47:371-379; Mamo et al., Clin Infect Dis, (2018), 66:1705-1711). (III) The therapeutic design assesses if post-Txp Bifido FMT can mitigate immunosuppressant-induced gut dysbiosis and metabolic disruption following Txp. Specimens will be collected longitudinally post-Txp; baseline samples taken prior to Txp and FMT to enable temporal tracking microbial and metabolic shifts. Tissues harvested include intestine, mesenteric and peripheral LNs, spleen, heart, intestinal lumen contents (via mucosa scraping), and serum. Tissues will be assessed by flow cytometry, immunohistochemistry (IHC), and RNA-seq; feces are for microbiome and metabolome; and sera are for metabolome and alloantibody. Intestinal permeability will be measured by FITC-Dextran. Time points: Mice will be euthanized on Day 0 (baseline, after abx, prior to Txp and FMT), Day 3 (post-Txp, acute effects); Days 7 and 30 (intermediate effects), and Day 60 (chronic effects). Feces will be collected at baseline, twice weekly for 60 days post-Txp. Controls: All designs will include heat-inactivated Bifido FMT, vehicle-only FMT (no bacteria), and immunosuppressant-only (No-FMT) controls; FMT duration may be adjusted based on outcomes. Validation. FMT of fecal supernatant with only metabolite lysates (no bacteria) verifies if fecal metabolites alone replicate phenotypes observed with the live bacteria. Power calculation. Based on previous results, we calculated an effect size of 1.69 (Bromberg et al., JCI Insight, (2018), 3, doi:10.1172/jci.insight.121045). Using G*Power and following Cohen's principle for one-way ANOVA, a sample size of 9/grp will achieve 80% power and a 5% alpha level (Faul et al., Behav Res Methods, (2007), 39:175-191; Cohen, J., Lwrence Earlbaum Associates Publishers (1988)). 10/grp will be used to account for potential loss due to empiric 5-10% technical graft failure. Part 1. Define microbiome-induced metabolic reprogramming in cardiac transplantation.

Scientific premise. 1) We previously demonstrated microbial modulation of alloimmunity is not mediated by bacterial surface components (e.g., exopolysaccharides or pili), but by broad influence on gut microbiome and metabolic activities (FIG. 26, 27 and (Ma, B. et al., BMC Microbiol, (2023), 23:394)) (Gavzy, S. J. et al., Transplantation, (2024), doi:10.1097/TP.0000000000004939; Bromberg et al., JCI Insight, (2018), 3, doi:10.1172/jci.insight.121045). 2) Chronic immunosuppressant use failed to control graft vascular inflammation post-Txp and led to pronounced gut dysbiosis with disrupted metabolism (FIG. 25, 26 and (Wu, L. et al., bioRxiv, (2025), 2025.2001.2002.631100; Wu, L. et al. JCI Insight, (2025), 10:186505; Kensiski, A. et al., Clin Microbiol Rev, (2025), e0017824). Bifido FMT appeared to mitigate this dysbiosis, reducing pathobiont expansion and restoring key metabolic processes impaired during chronic immunosuppression post-Txp (FIG. 26, 27). 3) Microbiome-derived metabolites significantly modulate alloimmunity and graft outcomes (Mathewson, N. D. et al., Nat Immunol, (2016), 17:505-513; Wu, H. et al., J Am Soc Nephrol, (2020), 31:1445-1461; Haak, B. W. et al., Blood, (2018), 131:2978-2986; Riwes et al., Am J Transplant, (2018), 18:23-29; Michonneau, D. et al., Nat Commun, (2019), 10:5695). SCFAs stimulate DC, MP, CD8 T cells and γδ T cells (Schulthess, J. et al., Immunity, (2019), 50:432-445 e437; Yang, K. et al., J Exp Med, (2021), 218: doi:10.1084/jem.20201915; Dupraz, L. et al. Cell Rep, (2021), 36:109332; Luu, M. et al., Nat Commun, (2021), 12:4077; Smith, P. M. et al., Science, (2013) 341, 569-573). Secondary BAs signal Tregs, Th17, or DC in immune-mediated disorders (Paik, D. et al., Nature, (2022), 603:907-912; Li, W. et al., Cell Host Microbe, (2021), 29:1366-1377; Hu, J. et al., Cell Rep, (2021), 36:109726). Kynurenine promotes Foxp3+ Treg differentiation via the aryl hydrocarbon receptor on naive CD4+ T cells, suppresses Teff responses, and induces cytotoxic effects on activated T cells, limiting alloreactivity (Fallarino, F. et al., Transpl Immunol, (2006), 17:58-60; Sharma, M. D. et al., Blood, (2009), 113:6102-6111; Guillonneau, C. et al., J Clin Invest, (2007), 117:1096-1106). Polyamines modulate systemic and mucosal adaptive immunity by controlling T cell differentiation (Carriche, G. M. et al., J Allergy Clin Immunol, (2021), 147:335-348, e311; Puleston, D. J. et al., Cell, (2021), 184:4186-4202, e4120). 4) Clinically, metabolomics are utilized to identify biomarkers for tracking rejection stage, monitoring graft recovery, and assessing immunosuppressant toxicity (Naesens et al., Nat Rev Nephrol, (2010), 6:614-628; Bouhifd et al., J Appl Toxicol, (2013), 33:1365-1383; Mueller et al., Semin Immunopathol, (2011), 33, 185-199; Suhre, K. et al. J Am Soc Nephrol, (2016), 27:626-636). Examples include TMAO as a marker for renal medullary injury, fecal metabolites predicting post-liver Txp infection risk, dietary interventions improve outcomes in allogeneic hematopoietic stem cell transplantation, and SCFAs promote donor-specific kidney allograft tolerance (Klepacki et al., Clin Chim Acta, (2015), 446, 43-53; Lehmann, C. J. et al., Cell Host Microbe, (2024), 32:117-130; Riwes et al., Am J Transplant, (2018), 18:23-29; Wu, H. et al., J Am Soc Nephrol, (2020), 31:1445-1461). Our goal is to 1.1) investigate microbial-driven metabolic changes; and 1.2) establish causality by modulating target metabolites.

Part 1.1. Whole metagenomic sequencing will be employed for taxonomic and functional characterization of gut microbiome (as in FIG. 25 and previously (Ma, B. et al. Sci Rep, (2023), 13:1023; Ma, B. et al., BMC Microbiol, (2023), 23:394; Ma, B. et al., MBio, (2022), 13:e0129922; Ma, B. et al., Nat Commun, (2020), 11; 940). Briefly: 1) DNA extraction as previously validated (Ma, B. et al. Frontiers in microbiology, (2018), 9:2755; Ravel, J. et al., Proc. Natl. Acad. Sci. USA, (2011), 108:Suppl 1, 4680-4687); 2) Illumina NovaSeq 6000 S4 sequencing (107 reads per library). 3) QC and post-QC using validated bioinformatics pipeline developed by Ma (Co-PI) that includes quality assessment, diversity analyses, assembly, taxonomic and pathway profiling, and differential abundance analyses (Ma, B. et al. Sci Rep, (2023), 13:1023; Segata, N. et al., Genome Biol, (2011), 12:R60, doi:10.1186/gb-2011-12-6-r60; Ma, B. et al., BMC Microbiol, (2023), 23:394; Ma, B. et al., MBio, (2022), 13:e0129922; Ma, B. et al., Nat Commun, (2020), 11; 940; Kieser et al., PLOS Comput Biol, (2022), 18:e1009947; Franzosa, E. A. et al., Nat Methods, (2018), 15:962-968; Law et al., Genome Biol, (2014), 15:R29, doi:10.1186/gb-2014-15-2-r29; Paulson et al., Nat Methods, (2013), 10:1200-1202).

Metabolome measurement will include both untargeted and targeted metabolome (as in FIG. 27) and previously 9 Ma, B. et al., BMC Microbiol, (2023), 23:394). Briefly: 1) ultra-high pressure liquid chromatography (UPLC) to comprehensively survey metabolites (Han, J. et al., Anal Chem, (2015), 87:1127-1136; Han, J. et al., Electrophoresis, (2016), 37:3089-3100). Strict QC includes blanks and internal standards spaced evenly in every batch. 2) Targeted assays using UPLC and triple quadrupole mass spectrometry to achieve high sensitivity. Panel selection will be guided by prior studies, Prelim Data, and pathways identified in untargeted metabolome (Gao, Y. et al. Am J Physiol Gastrointest Liver Physiol, (2021), 320:G521-G530; Ghosh et al., Cell Mol Gastroenterol Hepatol, (2021), 11:1463-1482; Agakidou et al., Front Pediatr, (2020), 8, 602255; Nolan, L. S. et al., Nutrients, (2021), 13, doi:10.3390/nu13103604). Established assay panels for FAs, BA, amino acids metabolism, sphingolipid synthesis, are routinely used in our lab. Integrating insights from both approaches on independent platforms will ensure unbiased, precise metabolite quantification. Metabolite annotation will follow rigorous preprocessing and leverage comprehensive referencing databases (i.e., PubChem, KEGG, and HMDB) to annotate compounds, pathways and hierarchical classification, as previously (Kim, S. et al., Nucleic Acids Res, (2021), 49:D1388-D1395; Hattori et al., Nucleic Acids Res, (2010), 38, W652-656; Wishart, D. S. et al., Nucleic Acids Res, (2018), 46, D608-D617; Ma, B. et al., BMC Microbiol, (2023), 23:394). Response variable modeling will combine PCA and multivariate partial-least-squares discriminant analysis (PLS-DA) to identify global differences between FMT groups and key discriminating metabolites (as in FIG. 27, previously) (Ma, B. et al., BMC Microbiol, (2023), 23:394; Le Cao et al., BMC Bioinformatics, (2011), 12:253, doi:10.1186/1471-2105-12-253). Pathway enrichment will be used to construct networks of overrepresented metabolites, enabling the identification of group-specific biochemical interactions, or “Metabotype” (Beger, R. D. et al., Metabolomics (2016), 12:149). Microbiome and metabolome correlation will use species-specific metabolic pathways by Human Microbiome Project Unified Metabolic Analysis Network and as previously, to identify microbial groups responsible for specific metabolic activities (Beghini, F. et al., Elife, (2021), 10:doi:10.7554/eLife.65088; Ma, B. et al., BMC Microbiol, (2023), 23:394). Time-series analyses will assess temporal changes from baseline, in microbial diversity, taxa abundance and metabolic pathways (as FIG. 26A and) (Gonzalez et al., BioData Min, (2012), 5:19, doi:10.1186/1756-0381-5-19). Mix-effects models and change-point detection will be used to identify significant within-subject shifts corresponding to phenotypic changes, as previously (Ravel, J. et al., Microbiome, (2013), 1:29, doi:10.1186/2049-2618-1-29; Gajer, P. et al., Sci Transl Med (2012), 4:132ra152).

Part 1.2. To establish causality, we will use two approaches based on pathway complexity, redundancy, and available intervention tools. For compounds like SCFAs or TMAO, direct intervention strategies will be employed as esblished201-203; for complex networks like polyamines, we will implement a pathway topology-informed agonist-antagonist design to leverage metabolic pathway knowledge: 1) (Target Blockade) Bifido FMT plus inhibition of polyamine synthesis. Two Txp groups treated with Bifido or no FMT on day 0 will also receive α-DLdifluoromethylornithine (DMFO, 20 mg/kg/d p.o.×7d), an irreversible inhibitor of ornithine decarboxylase (ODC) that is the rate limiting enzyme for putrescine synthesis (Somani et al., Mini Rev Med Chem, (2018), 18:1008-1021; Seckute, J. et al., Protein Sci, (2011), 20:1836-1844; Shirahata et al., Biochem Pharmacol, (1993), 45, 1897-1903). It is anticipated the inhibitor will prevent the Bifido effects, and grafts will have increased inflammation and fibrosis, DMFO may cause a phenotype in the no FMT group similar to Desulfo FMT. 2) (Supplement Bypass) Bifido FMT plus DMFO plus polyamine. We posit that DMFO enzyme inhibition will be overcome by adding the end products to bypass ODC. Txp groups will be treated as above plus putrescine or spermidine (30 mM in drinking water for 7 days), to test if polyamines can overcome DMFO blockade and restore Bifido effect. 3) (Supplement Rescue) Desulfo FMT plus polyamine. The hypothesis is that the polyamines will prevent Desulfo-induced dysbiosis and metabolic derangement. Txp mice treated with Desulfo or no FMT on day 0 will receive putrescine or spermidine (30 mM) to test if polyamines can restore Bifido effect. Depending on the results, doses may be adjusted, and additional targets may be added.

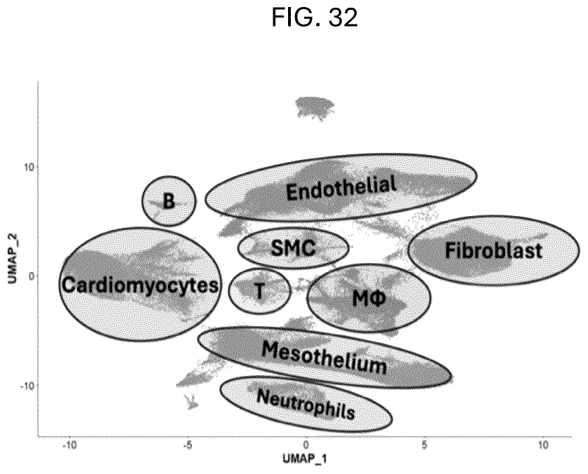

We expect to elucidate the metabolic mechanisms by which bacterial determinants modulate alloimmune responses that determine cardiac allograft outcomes, and to identify modifiable, mechanistically linked features critical for transplant success. 1) Bifido FMT will restore gut eubiosis (enriched “fermentators”, reinstate microbial diversity) and enhance key metabolic pathways disrupted during chronic immunosuppression (e.g., SCFAs, polyamines, kynurenine), resulting in preserved vascular integrity and graft function. 2) Through targeted interventions, we anticipate that polyamine supplementation, particularly with spermidine, will reproduce Bifido effects, while inhibition abrogates them, thereby establishing key mediators of microbiome-driven alloimmune responses. 3) Bifido FMT-induced changes in the gut are expected to produce a measurable “metabolic imprint” in the systemic circulation, including increased levels of spermidine and SCFAs, and reduced pro-atherogenic TMAO. 4) Early divergence in gut metabolic signatures is expected to predict long term graft outcomes, with Bifido FMT driving a tolerogenic profile and Desulfo FMT exacerbating dysbiosis and graft inflammation. Critically, microbial and metabolic shifts are expected to precede structural allograft injury, thus serving as early biomarkers and mechanistic drivers of CAV development. To assess therapeutic potential, additional experiments will test whether FMT administered at later time points (e.g., 30-day post-transplant) can reverse established CAV pathology. Results will guide the development of microbiome-informed interventions designed to intervene early in the post-transplant period, thus preventing or potentially reversing progression toward irreversible vascular and graft damage. 5) To evaluate tolerance, results will be validated with alternative immunosuppression. Instead of tacrolimus, Bifido and Desulfo will be assessed for their impact on tolerance using suboptimal (anti-CD40L, 250 μg day 0) or tolerogenic [CTLA4Ig (100 μg)+anti-CD40L (250 μg) day 0] immunosuppression. The results will determine if Bifido can induce tolerance or Desulfo can impair tolerance. Part 2. Microbiome-mediated alloantigen-specific responses in lymphoid tissue and grafts. Experimental Design. Using the design in FIG. 31, mice txp recipients will also be adoptively transferred with TEa T cell receptor (TCR) transgenic (Tg) CD4+ T cells (recognize donor I-E^{d} presented by recipient I-A^{b}) plus 2C TCR Tg CD8+ T cells (recognize donor H-2L^{d}) on day of Txp. We have strains, reagents, and experience to track these cells (Li, L. et al., J Clin Invest, (2022), 132, doi:10.1172/JCI156994). LNs, spleens and hearts will be assessed by flow cytometry and IHC for distribution and differentiation of CD4 and CD8 T cells and sera assessed for specific alloantibody. Mesenteric and systemic LNs, spleens and grafts will use snRNASeq to discern T and B cell immune responses. snRNAseq allows sequential tissue collection and preservation over multiple days with batch processing of nuclei, thereby reducing experimental variation while accommodating our longitudinal study design. As previous²¹³⁻²¹⁵ and FIG. 32, we will assess LNs, spleens and grafts to discern T cell subsets (CD4, CD8, Tregs, Th17, naïve, memory, activated) and B cells (B220, Ig isotype, resting, activated, immature, mature), their proportions and differentially expressed genes (DEGs). Empirically we expect ~1000 nuclei/μl sn suspension for library, sequenced at ~200 million reads/sample that yields >8,000 nuclei/sample (Li, L. et al., J Clin Invest, (2022), 132, doi:10.1172/JCI156994; Rousselle, T. V. et al., Sci Rep, (2022), 12:9851; Zhao, J. et al., J Clin Invest, (2022), 132, doi:10.1172/JCI159672). Bioinformatics analysis. We will use our established bioinformatics pipeline that incorporates preprocessing (Cell Ranger), analyses (Seurat), DEGs of intra- and inter-cell cluster (MAST), ligand-receptor analyses, Correlation Network Analyses, pathway enrichment, and trajectory analysis (Monocle 3) (Zheng, G. X. et al., Nat Commun, (2017), 8:14049; Satija et al., Nat Biotechnol, (2015), 33:495-502; Finak, G. et al., Genome Biol, (2015), 16:278; Armingol et al., Nat Rev Genet, (2021), 22, 71-88; Efremova et al., Nat Protoc, (2020), 15, 1484-1506; Yu et al., Bioinformatics, (2015), 31; 608-609; Van den Berge, K. et al., Nat Commun, (2020), 11:1201). gEAR (gene Expression Analysis Resource), a cloud-based portal developed internally, will be used for data visualization (Orvis, J. et al., Nat Methods, (2021), 18:843-844).

Part 2.1. Role of polyamines to regulate alloimmune responses and determine Treg/Teff ratios. Mice will be exposed to bacteria and/or metabolites (1.2 design) with TCR Tg T cell transfers. LNs, spleens and grafts will be assessed for T cell phenotype by IHC, flow cytometry, and snRNASeq. In vivo studies will be complemented with in vitro mechanistic probes of CD4+ or CD8+ T cells. Specifically, isolated naïve T cells will be incubated with graded doses of metabolites, their precursors, and/or enzyme inhibitors (i.e., S-(5′-adenosyl)-L-methionine iodide, DMFO, L-arginine, L-ornithine, putrescine, spermidine, spermine). Doses will be adjusted via cell viability and proliferation controls to determine direct effects of the drugs on T cells. We will determine effects on proliferation, death, differentiation (Th/Tc1, Th/Tc2, Th/Tc17, Treg), and migration when stimulated with anti-CD3/anti-CD28, alloantigen, or chemokines. Novel in vitro results will be confirmed by flow, IHC and snRNASeq on tissues from the in vivo experiments above. We have expertise in all these assays (Kumamoto et al., Proc Natl Acad Sci USA, (2011), 108:8749-8754; Warren et al., J Clin Invest, (2014), 124:2204-2218; Katakai et al., J Exp Med, (2004), 200:783-795; Van Vliet et al., J Histochem Cytochem, (1986), 34, 883-890; Simon et al., Trends Immunol, (2017), 38, 858-871; Geberhiwot et al., Journal of cell science, (2001), 114, 423-433; Simon, T. et al. Transplantation, (2019), 103:2075-2089). Part 2.2. Target metabolites regulate LN responses. We will test whether metabolites regulate LN structure. LNs will be obtained from groups treated as in Aim 2.1 and analyzed by IHC as previously for laminin α4:α5, distribution of T cells (CD4, CD8, Tregs, Teffs, TEa, 2C), B cells (B220, Ig, GL7, IgD), DCs (cDC, pDC), cytokines (IL-6, -7, -10, 33), chemokines (CCL21, CXCL9-12), adhesion molecules (VCAM-1, ICAM-1, MAdCAM-1), stromal fibers (collagen 1, ERTR7 (col VI), Pdpn) and vascular molecules (PNAd, CD31, Lyve-1, VEGFs) in various domains (cortex, medulla, HEV, CR, germinal centers, subcapsular) (Li, L. et al. J Clin Invest, (2020), 130, 2602-2619; Li, L. et al., J Clin Invest, (2022), 132, doi:10.1172/JCI156994).

We anticipate polyamines will be major determinants of LN remodeling, including laminin ratios and measures of cellular and molecular distribution which account for the tolerogenic versus inflammatory niches of the LN. Since the laminins are primarily regulated by FRC and the snRNASeq data can be mined for FRC subsets and phenotype, the data generated in part 2.1 will be evaluated for polyamine induced changes in FRCs. We anticipate that polyamines will result in changes in FRC subset proportions or DEGs in those subsets. Additional in vivo mechanistic confirmation will use our existing Cre-Lox strains to probe FRC, depending on which features are uncovered, e.g., laminin α4-flox, laminin α5-flox, LTβR-flox, CCL19-Cre and Pdgfrb2-Cre.

We anticipate target metabolites like polyamines will directly induce quiescent or homeostatic responses in lymphocytes and LNs. Further validation will include available mouse strains lacking specific enzymes for polyamine metabolism (Puleston, D. J. et al., Cell, (2021), 184:4186-4202, e4120). The results may suggest that a metabolic intervention, or “postbiotics”, in place of probiotic bacteria. It is likely that polyamines regulate DC or MØ responses that in turn regulate FRC; and blocking polyamines will result in inflammatory FRC, DC, or MΦ. The snRNASeq data may reveal local mesenteric and distant peripheral LN FRC, DC, and MP subsets, and markers of quiescence or inflammation.

Future experiments will confirm the DC and MF signatures with RT-PCR, IHC, and/or flow cytometry, and ultimately mechanistic studies with conditional KO strains and adoptive cell transfers to determine their roles in antigen presentation, rejection, or tolerance.

Part 3. Characterize the metabolic and alloimmune responses induced by FMT from human cardiac transplant recipients under immune regulatory or activated conditions. Scientific premise. 1) Emerging evidence shows significant association of the microbiome with promoting or inhibiting graft survival, yet it remains unclear if microbiome-mediated responses are casual, consequential or incidental. Identifying underlying mechanism and establishing causality are prerequisites for devising microbial-informed therapeutic strategies. Human microbiota-associated (HMA) animal models are key tools designed for such purposes and are more amenable to interventions than human studies (Arrieta et al., Cell Host Microbe, (2016), 19:575-578; Walter et al., Cell, (2020), 180, 221-232). However, germ-free HMA mice models are limited due to immature immune systems and aberrant inflammatory responses to FMT, and lack of translatability to model immune responses seen in transplant recipients with established microbiota (Round et al., Sci Immunol, 3, (2018), doi:10.1126/sciimmunol.aao1603; Milestones in human microbiota research, (2019)). HMA conventional mice models face challenges with certain human microbiota failing to colonize recipient mice. Staley et al. developed an antibiotic-conditioning regimen that allows sustained establishment of human microbiota in specific-pathogen-free (SPF) mice following a single gavage of cryopreserved microbiota (Staley, C. et al., Microbiome, (2017), 5:87). We adapted this design in our murine model of cardiac Txp chronic rejection, and showed the sufficiency of CAV patient-sourced FMT to modulate alloimmunity, graft fibrosis, and vascular inflammation (FIG. 30). 2) Despite differences between mouse and human microbiomes^{230,231}, their metabolic pathways are remarkably conserved, due to functional redundancy among different microbial taxa fulfilling similar functional roles (Monaco et al., BMC Evol Biol, (2015), 15:259; Tsaparas et al., BMC Evol Biol, (2006), 6:70; Wang, H. et al. Proc Natl Acad Sci USA, (2021), 118, doi:10.1073/pnas.2102344118; Nature 486, (2012), 207-214, doi:10.1038/nature11234). As demonstrated by ENCODE and related projects, metabolic pathways showed remarkable conservation between mice and humans, with 12,957 reactions and 8,347 metabolites shared, and only 67 reactions and 16 metabolites unique in either host (Wang, H. et al. Proc Natl Acad Sci USA, (2021), 118, doi:10.1073/pnas.2102344118; Stergachis, A. B. et al., Nature, (2014), 515:365-370; Cheng, Y. et al. Nature, (2014), 515:371-375; Uhlen, M. et al., Science, (2015), 347:1260419; Uhlen, M. et al., Sci Signal, (2019), 12: doi:10.1126/scisignal.aaz0274). This exceptional metabolic overlap offers a compelling basis for translating mechanistic insights from murine models to human metabolic pathways involved in alloimmune regulation. 3) Our goal is to leverage our HMA chronic rejection model to define microbiome-mediated metabolic mechanisms that are translatable to human alloimmune regulation and allograft outcomes.

Experimental design. Following our published methods and FMT protocols, experiments will be performed as described in Table 9 (Bokoliya et al., Front Cell Infect Microbiol, (2021), 11:711055; Barcena, C. et al., Nat Med, (2019), 25:1234-1242; Gavzy, S. J. et al., Transplantation, (2024), doi:10.1097/TP.0000000000004939; Ma, B. et al. Sci Rep, (2023), 13:1023; Bromberg et al., JCI Insight, 3, (2018), doi:10.1172/jci.insight.121045). Archived human fecal specimens from CAV and non-CAV patients (FIG. 29) will serve as FMT donors for murine Txp recipients. Healthy donor fecal samples from OpenBiome.org will serve as FMT control. Microbiome, metabolome, and immune assays, specimens, analyses are as described in Aims 1 and 2. HMA model incorporates a 6-day broad-spectrum abx-conditioning, a washout period, and weekly repeated FMT for four weeks to achieve sustained human microbiota colonization (Staley, C. et al., Microbiome, (2017), 5:87).

TABLE 8
Biobanked cardiac Txp patients ***
Table 1. Biobanked cardiac Txp patients***

| | Subj # | Age at Txp | Post-Txp Yrs | Ethnicity | Sex | BMI | CAV Grading** |
|---|---|---|---|---|---|---|---|
| CAV | C1 | 52 | 3.9 | AA | M | 29.5 | ISHLT CAV3 |
| | C2 | 50 | 2.6 | White | F | 24.0 | ISHLT CAV3 |
| | C3 | 47 | 13.2 | AA | M | 35.4 | ISHLT CAV2 |
| | C4 | 53 | 5 | AA | M | 32.6 | ISHLT CAV3 |
| | C5 | 38 | 10.6 | White | F | 31.6 | ISHLT CAV3 |
| | C6* | 60 | 3.8 | White | M | 28.4 | ISHLT CAV3 |
| | C7 | 54 | 1.3 | AA | M | 28.4 | ISHLT CAV2 |
| | C8 | 64 | 2.5 | AA | F | 20.9 | ISHLT CAV3 |
| | C9 | 61 | 5 | White | M | 31.6 | ISHLT CAV3 |
| | C10 | 53 | 1 | White | M | 32.1 | ISHLT CAV2 |
| non-CAV | N1 | 51 | 4.9 | White | F | 31.0 | ISHLT Grade 0 |
| | N2 | 69 | 7.2 | Other | M | 36.9 | ISHLT Grade 0 |
| | N3 | 70 | 0.5 | White | M | 27.6 | ISHLT Grade 0 |
| | N4 | 18 | 5 | AA | M | 26.1 | ISHLT Grade 0 |
| | N5* | 53 | 5.6 | White | M | 23.2 | ISHLT Grade 0 |
| | N6 | 57 | 0.9 | White | M | 24.2 | ISHLT Grade 0 |

*used in the preliminary human sourced FMT experiment (FIG. 9)
**assessed by coronary angiography and graft function according to the International Society for Heart and Lung Transplantation (ISHLT) classification
***fecal specimen cryopreserved for FMT experiments

TABLE 9
Specimen types and collection time points for experiments in FIG. 29.
Table 2. Specimen types and collection time points for experiments in FIG. 12.

| | | | | Feces*, ** | Sera*, ** | Harvest*, ** |
|---|---|---|---|---|---|---|
| | Abx | Tacrolimus: | FMT | d +0, 3, 7, 14, 21, | d −1, 14, | d +3, 7, 28, |
| Group | (d −6 to −1) | d 0 to +60 | d +0, 7, 14, 21 | 28, 35, 42, 49, 60 | 28, 42, 60 | 60 |
| Naïve ctrl | + | − | − | + | + | + |
| FMT ctrl | + | + | Healthy donor | + | + | + |
| CAV | + | + | CAV | + | + | + |
| Non-CAV | + | + | Non-CAV | + | + | + |
| Bac ctrl | + | + | Bifido/Desulfo | + | + | + |
| No bac ctrl | + | + | Fecal lysates | + | + | + |

*Harvest time: Day 0 (baseline, after abx, prior to Txp and FMT), Day 3 (post-Txp, acute effects); Days 7 and 28 (intermediate responses), and Day 60 (chronic impact).
** Specimens at euthanasia: intestine, mesenteric and peripheral LNs, spleen, heart, intestinal contents from scraping intestinal mucosa, and sera. FITC-dextran gavage prior to euthanasia to measure intestinal permeability. Baseline feces collected before FMT on Day 0 and Sera on day −1, and longitudinally post-Txp.

The abx regimen achieves effective gut decontamination without histological disruption, with no added benefit from more prolonged exposure (Liu, T. et al. BMC Microbiol, (2024), 24:283; Amorim, N. et al. Front Med (Lausanne), (2022), 9, 770017). The washout period separates abx and FMT steps. Naïve controls will verify that abx alone do not cause histological disruption and the observed phenotype is microbiome-mediated. This design is clinically relevant, reflecting regimens where antibiotic preconditioning precedes Txp and interventions. Bifido and Desulfo FMT will serve as internal bacterial controls.

### Validation

Longitudinal microbiome profiling will confirm consistent donor engraftment and track microbiome changes pre- and post-abx and throughput the FMT course. Additionally, FMT using fecal supernatant (metabolite lysates with no bacteria control) will determine if metabolites alone can replicate the phenotype observed with live microbiota. Power calculation. HMA models are relatively new and lack standardized power calculation (Walter et al., Cell, (2020), 180, 221-232). Empirically, small numbers of human donors (1-5) are used, each donor with 6-12 recipient mice (Walter et al., Cell, (2020), 180, 221-232). To control for interindividual variability, we will initially use 5 CAV and 5 non-CAV donors with 10 mice per group, then evaluate variability in microbiome and adjust sample size post hoc.

We anticipate that the HMA experiments will yield clinically translatable insights into how microbiome-mediated metabolic and immunologic mechanisms that causally influence alloimmunity and graft pathology, addressing previously unexplained variations in transplant outcomes. Specifically, 1) CAV-FMT is expected to induce pronounced dysbiosis (reduced diversity, pathobionts expansion, depletion of Lachnospiraceae and Bifidobacterium) and a distinct systemic metabolic “fingerprint”, marked by elevated TMAO, reduced SCFAs and spermidine, and pro-inflammatory shifts in tryptophan metabolism (e.g., quinolinic acid vs. tolerogenic kynurenine). 2) Immunologically, CAV-FMT will heighten alloimmune activation, reduce Treg induction, and skew DC toward a pro-inflammatory phenotype in LNs and graft, resulting in significant graft inflammation and fibrosis. Mechanistic validation using adoptive transfer of TCR Tg TEa and 2C cells (as in Aim 2) for antigen-specific responses and detailed analysis of Treg induction and distribution in LNs and grafts will further confirm the causal relationship. Collectively, these results will provide proof-of-concept that gut microbiome-driven metabolic dysregulation promotes systemic inflammation, vascular fibrosis, immunemediated graft injury, with changes that precede and correlate with progressive allograft pathology contributing to CAV development. 3) In contrast, non-CAV FMT is expected to confer partial protection, with enhanced Treg induction, reduced vascular inflammation, and a less inflammatory metabolic profile compared to CAV-FMT, although not fully matching the regulatory phenotype of Bifido or healthy control FMT. Longitudinal monitoring of microbial and metabolic changes, combined with immune and histopathological endpoints, will inform biomarkers and therapeutic candidates. We expect that early divergence in gut microbial and metabolic signatures will yield critical biomarkers predictive of subsequent graft pathology. Follow-up studies will examine whether these early biomarkers progress toward high grade CAV pathology and whether timely intervention targeting these gut signatures can delay or reverse vascular inflammation, sustain immune quiescence, and enhance graft survival.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A method of reducing transplant rejection in a subject undergoing an immunosuppressant therapy, comprising
i) detecting a dysbiotic state in the subject's gut microbiota resulting from the immunosuppressant therapy; and
ii) administering to the subject a therapy that attenuates the dysbiotic state in the subject's gut microbiota.

2. (canceled)

3. The method of claim 1, wherein detecting the dysbiotic state comprises analyzing the gut microbiota for an increase, relative increase, decrease, or relative decrease in one or more microbes.

4. The method of claim 1, wherein the subject's gut microbiota is analyzed for a relative increase in the presence of one or more pathobionts.

5. (canceled)

6. The method of claim 1, wherein detecting the dysbiotic state comprises analyzing the subject's metabolome profile for an increase, relative increase, decrease, or relative decrease in one or more metabolites.

7. The method of claim 1, wherein the detecting comprises detecting the presence, absence or relative amounts of one or more differentially expressed genes.

8. The method of claim 1, wherein the detecting comprises analyzing the lymph nodes in the subject.

9. (canceled)

10. (canceled)

11. (canceled)

12. The method of claim 1, wherein the immunosuppressive therapy comprises one or more immunosuppressive agent(s) selected from tacrolimus, mycophenolate mofetil, mycophenolic acid, azathioprine, prednisone, fingolimod, rapamycin, cyclosporine, sirolimus, everolimus, and belatacept.

13. (canceled)

14. (canceled)

15. The method of claim 1, wherein the transplant is a cardiac allograft transplant or kidney allograft transplant.

16. (canceled)

17. The method of claim 1, wherein the therapy that attenuates the dysbiotic state in the subject's gut microbiota comprises administration of pro-tolerogenic microbes.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. The method of claim 1, wherein the therapy that attenuates the dysbiotic state in the subject's gut microbiota comprises an effective amount of a metabolomic composition.

30. (canceled)

31. (canceled)

32. (canceled)

33. (canceled)

34. A method of reducing transplant rejection in a subject undergoing an immunosuppressant therapy, comprising
i) detecting a dysbiotic state in the subject's gut microbiota resulting from the immunosuppressant therapy; and
ii) stopping the immunosuppressant therapy in the subject and administering to the subject a different immunosuppressant therapy.

35. (canceled)

36. The method of claim 34, further comprising administering a therapy to the subject to attenuate the dysbiotic state in the subject's gut microbiota.

37. The method of claim 34, further comprising detecting the presence or absence of a dysbiotic state in the subject's gut microbiota after administering the different immunosuppressant therapy or the therapy to attenuate the dysbiotic state in the subject's gut microbiota.

38. The method of claim 34, wherein detecting the dysbiotic state comprises analyzing the gut microbiota for an increase, relative increase, decrease, or relative decrease in one or more microbes.

39. The method of claim 34, wherein the subject's gut microbiota is analyzed for a relative increase in the presence of one or more pathobionts.

40. (canceled)

41. The method of claim 34, wherein detecting the dysbiotic state comprises analyzing the subject's metabolome profile for an increase, relative increase, decrease, or relative decrease in one or more metabolites.

42. The method of claim 34, wherein the detecting comprises detecting the presence, absence or relative amounts of one or more differentially expressed genes.

43. The method of claim 34, wherein the detecting comprises analyzing the lymph nodes in the subject.

44. (canceled)

45. (canceled)

46. (canceled)

47. (canceled)

48. (canceled)

49. (canceled)

50. The method of claim 34, wherein the transplant is a cardiac allograft transplant or kidney allograft transplant.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A method of reducing transplant rejection in a subject undergoing an immunosuppressant therapy, comprising administering to the subject an effective amount of pro-tolerogenic microbes that attenuate a dysbiotic state in the subject's gut microbiota or an effective amount of a metabolomic composition that attenuates a dysbiotic state in the subject's gut microbiota.

69. (canceled)

70. (canceled)


SEQUENCE LISTING (2 sequences)

SEQ ID NO: 1 - DNA, 20 bp, synthetic construct
  source 1..20: mol_type = other DNA
agagtttgatcctggctcag

SEQ ID NO: 2 - DNA, 18 bp, synthetic construct
  source 1..18: mol_type = other DNA
cattaccgcggctgctgg